(19) United States
(12) Patent Application Publication
(10) Pub. No.: US 2025/0346647 A2
(43) Pub. Date: Nov. 13, 2025

(54) Optimized GIP Peptide Analogues

(71) Applicant: Antag Therapeutics ApS, Copenhagen N (DK)

(72) Inventors: Mette Marie ROSENKILDE, Hellerup (DK); Alexander Hovard SPARRE-ULRICH, Copenhagen (DK); Ditte RIBER, Brønshøj (DK); Samra Joke SANNI, Frederiksberg (DK)

(21) Appl. No.: 17/776,976

(22) PCT Filed: Dec. 3, 2020
(86) PCT No.: PCT/EP2020/084487

§ 371 Date: May 13, 2022

(30) Foreign Application Priority Data
Dec. 3, 2019 (WO) "PCT/EP2019/083506"
Jun. 10, 2020 (EP) 20179259.5

(51) Int. Cl. C07K 14/645; A61K 38/00

(52) U.S. Cl. CPC C07K 14/645; A61K 38/00

(57) ABSTRACT

Disclosed are glucose-dependent insulinotropic peptide (GIP)-derived peptide analogues which are antagonists of the GIP receptor. These GIP peptide analogues are optimized by comprising amino acid substitutions A13Aib and/or N24E, and are fatty acid conjugated with/without a linker, so to have improved solubility and/or physical stability.

### TECHNICAL FIELD

The present invention relates to glucose-dependent insulinotropic peptide (GIP)—derived peptide analogues, which are antagonists of the GIP receptor. These GIP peptide analogues are optimized by comprising amino acid substitutions A13Aib and/or N24E, and are fatty acid conjugated with/without a linker, so to have improved solubility and/or physical stability, while retaining or even improving the antagonistic effect at the GIP receptor.

### BACKGROUND

Glucose-dependent insulinotropic peptide (GIP) is a hormone secreted from the K cells of the gut following a meal¹. Like its sister hormone glucagon-like peptide 1 (GLP-1), GIP is a potent insulin secretagogue². In contrast to the glucagonostatic effect of GLP-1^{3, 4}, GIP has been shown to display glucagon-releasing properties under certain conditions (^{3, 5-13}). The interest in understanding the biology of GIP was intensified by the association between rodent GIPR (GIP receptor) and adiposity¹⁴⁻²¹. In humans, although less clear, there is likewise evidence for a role of GIP in fat metabolism with the demonstration of the GIPR expression in adipose tissue²², an association between high BMI and increased GIP levels^{22, 23} increased adipose tissue blood flow and TAG (triacylglycerol) deposition following GIP administration in a state of high insulin and high glucose²⁴, decreased basal and postprandial GIP levels observed in obese children put on a diet²⁵, and increased fasting GIP levels observed in healthy young men put on a high fat diet²⁶.

Thus, in addition to the general demand from researchers who witnessed the advances in the understanding of GLP-1 following the discovery of the GLP-1 receptor antagonist, exendin(9-39)^{27, 28}, the potential as an anti-obesity agent has attracted additional attention for the development of potent GIPR antagonists. Many different strategies have been undertaken in order to antagonize GIP's function, e.g. a small molecule receptor antagonist²⁹, immunization against GIP³⁰⁻³² various truncations and mutations of the GIP molecule with antagonistic properties³³⁻³⁹ and recently a potent antagonist antibody against the GIPR⁴⁰.

Under physiological conditions the 42 amino acid hormone, GIP, is degraded by the enzyme dipeptidylpeptidase 4 (DPP-4), which cleaves at the third position of the GIP molecule to yield GIP(3-42). Synthetic porcine GIP3-42 displayed no antagonist properties in pigs or perfused rat pancreata in physiological concentrations while in vitro it antagonized the human GIPR⁴¹. Many peptide hormones are post-translationally modified resulting in various biological forms with different lengths and amino acid modifications^{42,43}. Thus, it has been shown that GIP(1-30) is produced as a result of post-translational processing⁴⁴ and that it is an agonist on the GIPR^{33, 45}. If GIP(1-30) is secreted into the circulation in humans, the cleavage catalyzed by DPP-4 would result in GIP(3-30). The sequence of native GIP(3-30) is

(SEQ ID NO: 68)
EGTFISDYSIAMDKIHQQDFVNWLLAQK.

GIP(3-30) is however poorly soluble at neutral pH around 7.5 and as such not suitable for pharmaceutical administration.

On the basis of that, there is a need for GIP peptide analogues that, in addition to having satisfactorily high antagonistic activity at the GIP receptor, are sufficiently soluble (especially at physiological pH, such as pH around 7.5, where GIP(3-30) is not) and stable, such as physically stably, in aqueous liquid medium. These analogues may advantageously be provided in the form of a ready-to-use liquid pharmaceutical formulation adapted for immediate injection and may be able to be stored for a satisfactorily long period of time prior to use.

### SUMMARY

The present inventors have identified acylated GIP peptides which comprise amino acid substitutions A13Aib and/or N24E and which are antagonists of the GIPR that surprisingly result in optimized properties such as improved solubility and/or physical stability as well as retained or even improved antagonistic properties. This makes them potentially useful in a range of therapeutic applications.

The GIP peptides of the present disclosure are N-terminally truncated compared to native GIP(1-42) and at least do not comprise the first two amino acids in position 1 and 2 of GIP(1-42).

In one aspect, the present disclosure relates to a glucose-dependent insulinotropic peptide (GIP) analogue consisting of amino acid sequence SEQ ID NO:1:

3 - 4 - 5 - 6 7 8 9 10 11 12 13 14 15 16 17
X₁- G - T - F - I - S - D - Y - S - I - A - M - D - K - I
18 19 20 21 22 23 24 25 26 27 28 29 30
H - Q - Q - D - F - V - N - W - L - L - A - Q - K -Z,

wherein X₁ is any amino acid or omitted;
or a functional variant thereof, wherein said variant has 1 to 8 individual amino acid substitutions at any amino acid of SEQ ID NO:1,
wherein N at position 24 of SEQ ID NO:1, or a functional variant thereof, is substituted with E and/or wherein A at position 13 of SEQ ID NO:1, or a functional variant thereof, is substituted with 2-Aminoisobutyric acid (Aib),
wherein Z is a peptide comprising one or more amino acid residues of the C-terminus of Exendin-4(31-39) (PSSGAPPPS; SEQ ID NO:67; CE31-39) or is omitted, and wherein said peptide is modified by attaching one fatty acid molecule at one amino acid residue at any position of SEQ ID NO 1 or said functional variant thereof or at one amino acid residue at any position of Z SEQ ID NO:67; CE31-39.

An important advantage of the above aspect, where GIP peptide analogues comprise amino acid substitutions A13Aib and/or N24E is that the solubility and/or stability is improved as compared to e.g. native GIP(3-30).

Improved solubility may comprise or constitute improved solubility compared to e.g. GIP(3-30) at pH 7 (e.g. in 50 mM phosphate buffer at pH 7), pH 7.5 (e.g in 50 mM phosphate buffer at pH 7.5), pH 8 (e.g. in MilliQ water at pH 8) and/or at pH 8.5 (e.g. in MilliQ water at pH 8.5). The determination may be performed under the conditions set forth in “Assessment of solubility”. A solubility of more than 1 mg/ml or 5 mg/ml or more than 7.5 mg/ml, more than 10 mg/ml, or even more than 15 mg/ml may be desirable.

Improved stability may comprise or constitute improved physical stability and/or improved chemical stability as e.g. compared to GIP(3-30).

Improved physical stability may comprise or constitute reduced tendency to aggregate, e.g. to form either soluble or insoluble aggregates, e.g. fibrils. Aggregation (e.g. fibril formation) may be determined, for example, at a starting concentration of 1 mg/ml dissolved peptide at pH 7.5 and 25 degrees Celsius. An appropriate time period may be used, e.g. 24 hours, 50 hours or 96 hours. Aggregation may be determined under the conditions set out in “Assessment of physical stability”, with or without agitation. No fibrils detected within 96 hours with agitation may be desirable.

A further important advantage of the above aspect is that the antagonistic effect of the GIP peptide analogues are preferably retained or even improved. This may especially be the case where GIP peptide analogues comprise amino acid substitutions A13Aib.

In another aspect, the invention relates to the use of such GIP peptide analogues as a medicament.

In yet another aspect, the invention relates to the use of such GIP peptide analogues in a method of treating a condition selected from the group consisting of metabolic syndrome, obesity, pre-diabetes, type I diabetes, type 2 diabetes, insulin resistance, elevated fasting glucose, hyperglycemia, elevated fasting serum triglyceride levels, low levels of very low-density lipoprotein (VLDL), low high-density lipoprotein (HDL) levels, dyslipidemia, increased/decreased low-density lipoprotein (LDL), high cholesterol levels, abnormal deposition of lipids, a cardiovascular disease, elevated blood pressure and atherosclerosis.

### DESCRIPTION OF DRAWINGS

FIG. 1. Comparison between a reference GIP analogue with lower physical stability (AT364 in phosphate buffer—FIG. 1A), that forms fibrils, and a GIP peptide analogue with high physical stability (AT763 in phosphate buffer—FIG. 1B) that does not form fibrils. Note that both curves start at 0-40 absorbance units (AU), indicating no fibrils, but only in FIG. 1A increase in absorbance is observed, due to formation of fibrils. Note also the different scale on the Y-axis. The gap in the curves is due to an unfortunate issue related to a restart of the plate reader software about 16 hours after start of the measurements. The plate reader was subjecting the samples to orbital rotation during the entire measurement, even during the 16 h with data loss. The first 13 cycles were recorded and could be used to determine the pre-transitional baseline (see starting points). The measurement data collection was reinitiated and another 764 cycles were run for a total of about 94 h.

### DEFINITIONS

The term “affinity” refers to the strength of binding between a receptor and its ligand(s).

In the present context, affinity of a peptide antagonist for its binding site (Ki) will determine the duration of inhibition of agonist activity. The affinity of an antagonist can be determined experimentally using Schild regression on functional studies or by radioligand binding studies like 1) competitive binding experiments using the Cheng-Prusoff equation, 2) saturation binding experiments using the Scatchard equation or 3) kinetic studies with determination of on- and off rates (Kₒₙ and K_{off}, respectively).

The term “IC50” represents the half maximal inhibitory concentration (IC50), which is a measure of the effectiveness of a substance in inhibiting a specific biological or biochemical function. This quantitative measure indicates how much of a particular drug or other substance (e.g. antagonist) is needed to inhibit a given biological process (or component of a process, i.e. an enzyme, cell, cell receptor or microorganism) by half. It is commonly used as a measure of antagonist drug potency in pharmacological research. IC50 represents the concentration of a drug that is required for 50% inhibition in vitro. In the present context, the IC50 value can also refer to the concentration of a drug at which 50% of a radio labelled ligand is displaced from the receptor, which is a characterization of drug affinity done in competition binding experiments.

The term “agonist” in the present context refers to a peptide, or analogue thereof, capable of binding to and activating downstream signalling cascades from a receptor.

The term “antagonist” in the present context refers to a GIP peptide analogue as defined herein, capable of binding to and blocking or reducing agonist-mediated responses of a receptor. Antagonists usually do not provoke a biological response themselves upon binding to a receptor. Antagonists have affinity but no efficacy for their cognate receptors, and binding of an antagonist to its receptor will inhibit the function of an agonist or inverse agonist at receptors. Antagonists mediate their effects by binding to the active (orthosteric) site or to allosteric sites on receptors, or they may interact at unique binding sites not normally involved in the biological regulation of the receptor's activity. Antagonist activity may be reversible or irreversible depending on the longevity of the antagonist-receptor complex, which, in turn, depends on the nature of antagonist-receptor binding. The majority of drug antagonists typically achieve their potency by competing with endogenous ligands or substrates at structurally defined binding sites on receptors. Antagonists may be competitive, non-competitive, uncompetitive, silent antagonists, partial agonists or inverse agonists.

A competitive antagonist (also known as surmountable antagonist) reversibly binds to receptors at the same binding site (i.e. at the active site) as the endogenous ligand or agonist, but without activating the receptor. Agonists and antagonists thus “compete” for the same binding site on the receptor. Once bound, an antagonist blocks agonist binding. The level of activity of the receptor is determined by the relative affinity of each molecule for the site and their relative concentrations. High concentrations of a competitive antagonist will increase the proportion of receptors that the antagonist occupies.

The term “non-competitive antagonism” (also called nonsurmountable or insurmountable antagonism) describes two distinct phenomena with functionally similar results: one in which the antagonist binds to the active site of the receptor, and one in which the antagonist binds to an allosteric site of the receptor. Unlike competitive antagonists, which affect the amount of agonist necessary to achieve a maximal response but do not affect the magnitude of that maximal response, non-competitive antagonists reduce the magnitude of the maximum response that can be attained by any amount of agonist.

The term “silent antagonist” refers to a competitive receptor antagonist that has absolutely no intrinsic activity for activating a receptor.

The term “partial agonist” refers to an agonist that, at a given receptor, might differ in the amplitude of the functional response that it elicits after maximal receptor occupancy. Partial agonists can act as a competitive antagonist in the presence of a full agonist (or a more efficacious agonist), as it competes with the full agonist for receptor occupancy, thereby producing a net decrease in the receptor activation as compared to that observed with the full agonist alone.

The term “inverse agonist” refers to a ligand, such as a GIP peptide analogue, that is capable of binding to the same receptor binding site as an agonist and antagonize its effects. Furthermore, an inverse agonist can also inhibit the basal activity of constitutively active receptors.

The term “glucose-dependent insulinotropic polypeptide receptor (GIPR) antagonists” as used herein refers to a compound, such as a peptide, capable of binding to and blocking or reducing agonist-mediated responses of GIPR.

The term “Individual” refers to vertebrates, particular members of the mammalian species, preferably primates including humans. As used herein, ‘subject’ and ‘individual’ may be used interchangeably.

An “isolated peptide” is a peptide separated and/or recovered from a component of their natural, typically cellular, environment, that is essentially free from contaminating cellular components, such as carbohydrate, lipid, or other proteinaceous impurities associated with the polypeptide in nature. Typically, a preparation of isolated peptide contains the peptide in a highly purified form, i.e., at least about 80% pure, at least about 90% pure, at least about 95% pure, greater than 95% pure, or greater than 99% pure. The term “isolated” does not exclude the presence of the same peptide in alternative physical forms, such as dimers, tetramers or alternatively glycosylated or derived forms.

An “amino acid residue” can be a natural or non-natural amino acid residue linked by peptide bonds or bonds different from peptide bonds. The amino acid residues can be in D-configuration or L-configuration. An amino acid residue comprises an amino terminal part (NH₂) and a carboxy terminal part (COOH) separated by a central part comprising a carbon atom, or a chain of carbon atoms, at least one of which comprises at least one side chain or functional group. NH₂ refers to the amino group present at the amino terminal end of an amino acid or peptide, and COOH refers to the carboxy group present at the carboxy terminal end of an amino acid or peptide. The generic term amino acid comprises both natural and non-natural amino acids. Natural amino acids of standard nomenclature as listed in J. Biol. Chem., 243:3552-59 (1969) and adopted in 37 C.F.R., section 1.822(b)(2) belong to the group of amino acids listed herewith: Y,G,F,M,A,S,I,L,T,V,P,K,H,Q,E,W,R,D,N and C. Non-natural amino acids are those not listed immediately above. Also, non-natural amino acid residues include, but are not limited to, modified amino acid residues, L-amino acid residues, and stereoisomers of D-amino acid residues.

An “equivalent amino acid residue” refers to an amino acid residue capable of replacing another amino acid residue in a polypeptide without substantially altering the structure and/or functionality of the polypeptide. Equivalent amino acids thus have similar properties such as bulkiness of the side-chain, side chain polarity (polar or non-polar), hydrophobicity (hydrophobic or hydrophilic), pH (acidic, neutral or basic) and side chain organization of carbon molecules (aromatic/aliphatic). As such, “equivalent amino acid residues” can be regarded as “conservative amino acid substitutions”, and it is the substitution of amino acids whose side chains have similar biochemical properties and thus do not affect the function of the peptide.

Among the common amino acids, for example, a “conservative amino acid substitution” can also be illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine.

Within the meaning of the term “equivalent amino acid substitution” as applied herein, one amino acid may be substituted for another, in one embodiment, within the groups of amino acids indicated herein below:
i) Amino acids having polar side chains (Asp, Glu, Lys, Arg, His, Asn, Gln, Ser, Thr, Tyr, and Cys,)
ii) Amino acids having non-polar side chains (Gly, Ala, Val, Leu, lie, Phe, Trp, Pro, and Met)
iii) Amino acids having aliphatic side chains (Gly, Ala Val, Leu, lie)
iv) Amino acids having cyclic side chains (Phe, Tyr, Trp, His, Pro)
v) Amino acids having aromatic side chains (Phe, Tyr, Trp)
vi) Amino acids having acidic side chains (Asp, Glu)
vii) Amino acids having basic side chains (Lys, Arg, His)
viii) Amino acids having amide side chains (Asn, Gln)
ix) Amino acids having hydroxy side chains (Ser, Thr, Tyr)
x) Amino acids having sulphur-containing side chains (Cys, Met),
xi) Neutral, weakly hydrophobic amino acids (Pro, Ala, Gly, Ser, Thr)
xii) Hydrophilic, acidic amino acids (Gln, Asn, Glu, Asp), and
xiii) Hydrophobic amino acids (Leu, lie, Val)

In addition, a serine residue of a peptide of the present disclosure may be substituted with an amino acid selected from the group consisting of Gln, Asn and Thr (all amino acids with polar uncharged side chains); and independently thereof, a glycine residue (Gly) is substituted with an amino acid selected from the group consisting of Ala, Val, Leu, and Ile; and independently thereof, an arginine residue (Arg) is substituted with an amino acid selected from the group consisting of Lys and His (all have positively charged side chains); and independently thereof, a lysine residue (Lys) may be substituted with an amino acid selected from the group consisting of Arg and His; and independently thereof, a methionine residue (Met) may be substituted with an amino acid selected from the group consisting of Leu, Pro, lie, Val, Phe, Tyr and Trp (all have hydrophobic side chains); and independently thereof, a glutamine residue (Gln) may be substituted with an amino acid selected from the group consisting of Asp, Glu, and Asn; and independently thereof, an alanine residue (Ala) may be substituted with an amino acid selected from the group consisting of Gly, Val, Leu, and Ile.

Where the L or D form (optical isomers) has not been specified it is to be understood that the amino acid in question has the natural L form, cf. Pure & Appl. Chem. Vol. (56(5) pp 595-624 (1984) or the D form, so that the peptides formed may be constituted of amino acids of L form, D form, or a sequence of mixed L forms and D forms.

As used herein, a Glutamic acid (Glu) mimetic is a moiety, with two carboxy functional groups separated by three carbon atoms. Examples are beta-Glu, gamma-Glu or glutaric acid. Glutaric acid is also known as Pentanedioic acid.

A “functional variant” of a peptide is a peptide capable of performing essentially the same functions as the peptide it is a functional variant of. In particular, a functional variant can essentially bind the same molecules, such as receptors, or perform the same receptor mediated responses as the peptide it is a functional variant of. A functional variant of a “glucose-dependent insulinotropic peptide (GIP) analogue” is a peptide, that can bind to the GIPR and either activate or inhibit GIPR downstream signalling, such as cAMP generation. A functional variant of a glucose-dependent insulinotropic peptide receptor (GIPR) antagonist is a peptide, that can bind to the GIPR and inhibit or reduce agonist-mediated GIPR signalling, such as cAMP generation.

A “bioactive agent” (i.e. a biologically active substance/agent) is any agent, drug, compound, composition of matter or mixture which provides some pharmacologic, often beneficial, effect that can be demonstrated in vivo or in vitro. It refers to the GIP peptide analogues as defined herein and compounds or compositions comprising these. As used herein, this term further includes any physiologically or pharmacologically active substance that produces a localized or systemic effect in an individual.

The terms “drug” and “medicament” as used herein include biologically, physiologically, or pharmacologically active substances that act locally or systemically in the human or animal body.

The terms “treatment” and “treating” as used herein refer to the management and care of a patient for the purpose of combating a condition, disease or disorder. The term is intended to include the full spectrum of treatments for a given condition from which the patient is suffering, and refer equally to curative therapy, prophylactic or preventative therapy and ameliorating or palliative therapy, such as administration of the peptide or composition for the purpose of: alleviating or relieving symptoms or complications; delaying the progression of the condition, partially arresting the clinical manifestations, disease or disorder; curing or eliminating the condition, disease or disorder; amelioration or palliation of the condition or symptoms, and remission (whether partial or total), whether detectable or undetectable; and/or preventing or reducing the risk of acquiring the condition, disease or disorder, wherein “preventing” or “prevention” is to be understood to refer to the management and care of a patient for the purpose of hindering the development of the condition, disease or disorder, and includes the administration of the active compounds to prevent or reduce the risk of the onset of symptoms or complications. The term “palliation”, and variations thereof, as used herein, means that the extent and/or undesirable manifestations of a physiological condition or symptom are lessened and/or time course of the progression is slowed or lengthened, as compared to not administering compositions of the present invention.

The individual to be treated is preferably a mammal, in particular a human being. Treatment of animals, such as mice, rats, dogs, cats, cows, horses, sheep and pigs, is, however, also encompassed herewith.

An “individual in need thereof” refers to an individual who may benefit from the present disclosure. In one embodiment, said individual in need thereof is a diseased individual, wherein said disease may be a metabolic disease or disorder such as obesity or diabetes, a bone density disorder or a cancer.

A treatment according to the invention can be prophylactic, ameliorating and/or curative.

“Pharmacologically effective amount”, “pharmaceutically effective amount” or “physiologically effective amount” of a bioactive agent is the amount of a bioactive agent present in a pharmaceutical composition as described herein that is needed to provide a desired level of active agent in the bloodstream or at the site of action in an individual (e.g. the lungs, the gastric system, the colorectal system, prostate, etc.) to be treated to give an anticipated physiological response when such composition is administered. A bioactive agent in the present context refers to a GIP peptide analogue as disclosed herein.

“Co-administering” or “co-administration” as used herein refers to the administration of one or more GIP peptide analogues of the present invention and a state-of-the-art pharmaceutical composition. The at least two components can be administered separately, sequentially or simultaneously.

“Physical stability” as used herein refers to a measure of the tendency of a peptide (e.g. a GIP peptide analogue of the invention) to form soluble or insoluble aggregates of the peptide, for example as a result of the peptide to stresses and/or interaction with interfaces and surfaces that are destabilizing, such as hydrophobic surfaces and interfaces. Physical stability of aqueous peptide solutions may be evaluated by means of visual inspection and/or turbidity measurements after exposing the composition, filled in suitable cartridges (e.g. cartridges or vials), to mechanical/physical stress (e.g. agitation) for various time periods. A composition may be classified as physically unstable with respect to peptide aggregation when it exhibits visual turbidity. Alternatively, the turbidity of a composition can be evaluated by simple turbidity measurements well-known to the skilled person. Physical stability of an aqueous peptide composition can also be evaluated by using an agent that functions as a spectroscopic probe of the conformational status of the peptide. The probe is preferably a small molecule that preferentially binds to a non-native conformer of the peptide. One example of such small-molecular spectroscopic probe is Thioflavin T, which is a fluorescent dye that has been widely used for the detection of amyloid fibrils. In the presence of fibrils, and perhaps also other peptide configurations, Thioflavin T gives rise to a new excitation maximum at about 450 nm and enhanced emission at about 482 nm when bound to a fibril form of the peptide. Unbound Thioflavin T is essentially non-fluorescent at the wavelengths in question.

### DETAILED DESCRIPTION

GIP refers to glucose-dependent insulinotropic polypeptide, also known as Gastric Inhibitory Peptide (or polypeptide). As used herein the abbreviation GIP or hGIP is human GIP (Uniprot accession number P09681). GIP is derived from a 153-amino acid proprotein and circulates as a biologically active 42-amino acid peptide. It is synthesized by K cells of the mucosa of the duodenum and the jejunum of the gastrointestinal tract.

GIPR (or GIP receptor) refers to gastric inhibitory polypeptide receptors. These seven-transmembrane proteins are found at least on beta-cells in the pancreas. As used herein the abbreviation GIPR or hGIPR is human GIPR (Uniprot accession number P48546).

In one embodiment, Exendin-4 is a peptide having amino acid sequence

(SEQ ID NO: 69)
HGEGTFTSDLSKQMEEEAVRLFIEWLKNGGPSSGAPPPS.

In one embodiment GIP(3-30) is a peptide having amino acid sequence

(SEQ ID NO: 68; GIP3-30)
EGTFISDYSIAMDKIHQQDFVNWLLAQK.

The present inventors have identified acylated GIP peptide analogues which comprise amino acid substitutions A13Aib and/or N24E and which are antagonists of the GIPR that surprisingly result in improved solubility and/or physical stability as well as retained or improved antagonistic properties. This makes them potentially useful in a range of therapeutic applications.

### GIP Peptides

### Substituted GIP Peptide Analogues

It is an aspect of the present disclosure to provide a glucose-dependent insulinotropic peptide (GIP) analogue consisting of amino acid sequence SEQ ID NO:1:

3 - 4 - 5 - 6 7 8 9 10 11 12 13
X₁ - G - T - F - I - S - D - Y - S - I - A -
14 15 16 17 18 19 20 21 22 23 24
M - D - K - I - H - Q - Q - D - F - V - N -
25 26 27 28 29 30
W - L - L - A - Q - K - Z,

wherein X₁ is any amino acid or omitted;
or a functional variant thereof, wherein said variant has 1 to 8 individual amino acid substitutions at any amino acid of SEQ ID NO:1,
wherein N at position 24 of SEQ ID NO:1, or a functional variant thereof, is substituted with E and/or wherein A at position 13 of SEQ ID NO:1, or a functional variant thereof, is substituted with 2-Aminoisobutyric acid (Aib),
wherein Z is a peptide comprising one or more amino acid residues of the C-terminus of Exendin-4(31-39) (PSSGAPPPS; SEQ ID NO:67; CE31-39) or is omitted, and
wherein said peptide is modified by attaching one fatty acid molecule at one amino acid residue at any position of SEQ ID NO 1 or said functional variant thereof or at one amino acid residue at any position of Z SEQ ID NO:67; CE31-39.

It is also an aspect of the present disclosure to provide a GIP analogue consisting of an amino acid sequence selected from the group consisting of:

```
(SEQ ID NO: 2)
  3 - 4 - 5 - 6 7 8 9 10 11 12 13
X₁ - G - T - F - I - S - D - Y - S - I - A -
14 15 16 17 18 19 20 21 22 23 24
M - D - K - I H - Q - Q - D - F - V - E -
25 26 27 28 29 30
W - L - L - A - Q - K - Z,,
(SEQ ID NO: 3)
  3 - 4 - 5 - 6 7 8 9 10 11 12 13
X₁ - G - T - F - I - S - D - Y - S - I -Aib -
14 15 16 17 18 19 20 21 22 23 24
M - D - K - I H - Q - Q - D - F - V - N -
25 26 27 28 29 30
W - L - L - A - Q - K - Z,,
and
(SEQ ID NO: 4)
  3 - 4 - 5 - 6 7 8 9 10 11 12 13
X₁ - G - T - F - I - S - D - Y - S - I -Aib -
14 15 16 17 18 19 20 21 22 23 24
M - D - K - I - H - Q - Q - D - F - V - E -
25 26 27 28 29 30
W - L - L - A - Q - K - Z,,
```

wherein X₁ is any amino acid or omitted;
or a functional variant thereof, wherein said variant has 1 to 8 individual amino acid substitutions at any amino acid of SEQ ID NO:2 (except E at position 24); of SEQ ID NO:3 (except Aib at position 13); and of SEQ ID NO:4 (except Aib at position 13 and E at position 24);
wherein Z is a peptide comprising one or more amino acid residues of the C-terminus of Exendin-4(31-39) (PSSGAPPPS; SEQ ID NO:67; CE31-39), and
wherein said peptide is modified by attaching one fatty acid molecule at one amino acid residue at any position of SEQ ID NO:2-4, or said functional variant thereof, or at one amino acid residue at any position of Z SEQ ID NO:67; CE31-39.

An important advantage of the above aspects, where position 13 is substituted with Aib and/or position 24 is substituted with E is that solubility and/or physical stability appear to be improved. When both position 13 is substituted with Aib and position 24 is substituted with E a superior or even synergistic improvement in solubility and/or physical stability may be obtained.

In one embodiment, the present disclosure provides a GIP peptide analogue as defined herein above, wherein said GIP peptide analogue is an antagonist of GIPR.

A GIP peptide which has been modified as compared to the native GIP peptide is referred to as a GIP peptide analogue. A GIP peptide analogue according to the present disclosure is preferably a GIPR antagonist.

In one embodiment the GIP peptide analogue, or a functional variant thereof, according to the present disclosure, is an isolated peptide.

In one embodiment of the present disclosure, the GIP peptide analogue has improved solubility as e.g. compared to a corresponding sequence without Aib in position 13 and/or E in position 24.

In one embodiment of the present disclosure, the GIP peptide analogue has improved solubility as compared to native GIP(3-30) and/or as compared to AT364 (SEQ ID NO:6; GIP(3-30) [H18K]C16diacid+Cex(31-39).

In one embodiment of the present disclosure, the GIP peptide analogue has improved solubility as compared to GIP(3-30) and/or as compared to AT364 (SEQ ID NO:6) at pH 7 to 9, such as at pH 7 to 8.5, such as at pH 7.0 to 8.0 or pH 7.5 to 8.5., such as measured via visual inspection or for example measured in a UV microplate, where a turbidity absorbance criterion for a peptide solubility of ≥1 mg/ml can be set as absorbance at 325 nm≤0.02 absorbance units (e.g. 5 to 6 times the standard deviation of 8 buffer samples in a plate).

In one embodiment, the GIP peptide analogue has an aqueous solubility of at least 1 mg/ml, such as at least 5 mg/ml, such as at least 7.5 mg/ml, such as at least 10 mg/ml, such as at least 15 mg/ml.

In one embodiment, the GIP peptide analogue has an aqueous solubility at pH between 7 to 9, such as around pH 7.5 or around 8, of at least 1 mg/ml, such as at least 5 mg/ml, such as at least 7.5 mg/ml, such as at least 10 mg/ml, such as at least 15 mg/ml.

In one embodiment the GIP peptide analogue has improved physical stability as measured by a fibrillation lag time in a ThT assay of more than about 24 hours, such as a fibrillation lag time in a ThT assay of more than about 50 hours, such as a fibrillation lag time in a ThT assay of more than about 96 hours, such as a fibrillation lag time in a ThT assay of more than about 168 hours.

In one embodiment the GIP peptide analogue does not form fibrils even when agitated within about 24 hours, such as within about 50 hours, such as within about 96 hours.

In one embodiment the GIP peptide analogue has improved physical stability at pH 7 to 8.5, such as around pH 7.5, as measured by a fibrillation lag time in a ThT assay of more than about 24 hours, such as a fibrillation lag time in a ThT assay of more than about 50 hours, such as a fibrillation lag time in a ThT assay of more than about 96 hours, such as a fibrillation lag time in a ThT assay of more than about 168 hours.

In one embodiment of the present disclosure, the GIP peptide analogue has improved physical stability as compared to GIP(3-30) and/or as compared to AT364 (SEQ ID NO:6) at pH 7 to 9, such as at pH 7 to 8.5, such as at pH 7.0 to 8.0 or pH 7.5 to 8.5., such as measured in an assay that determines aggregation, such as via a Thioflavin T (ThT) assay, an example of which is described in the section “Assessment of physical stability”.

In one embodiment of the present disclosure, the GIP peptide analogue is modified by attaching one fatty acid molecule at one amino acid residue at positions 3 to 29 of SEQ ID NO 1, or said functional variant thereof.

In one embodiment of the present disclosure there is provided a GIP peptide analogue selected from any one of SEQ ID NOs: 1-4, wherein:
the amino acid at position 3 is selected from E, glutaric acid, succinic acid and adipic acid;
the amino acid at position 9 is selected from D and E;
the amino acid at position 11 is selected from S, K and A;
the amino acid at position 12 is selected from I and K;
the amino acid at position 13 is selected from A, 2-Aminoisobutyric acid (Aib) and K;
the amino acid at position 14 is selected from M, L and Nle;
the amino acid at position 15 is selected from D and E;
the amino acid at position 16 is selected from K and R;
the amino acid at position 17 is selected from I and K;
the amino acid at position 18 is selected from H and K;
the amino acid at position 20 is selected from Q and K;
the amino acid at position 21 is selected from D and E;
the amino acid at position 24 is selected from N, Q and E;
the amino acid at position 34 if present is selected from P and K; and/or
the amino acid at position 40 if present is K or is absent.

In one embodiment of the present disclosure there is provided a GIP peptide analogue selected from any one of SEQ ID NOs: 1-4, wherein:
the amino acid at position 4 is G;
the amino acid at position 5 is T;
the amino acid at position 6 is F;
the amino acid at position 7 is I;
the amino acid at position 22 is F;
the amino acid at position 23 is V;
the amino acid at position 25 is W;
the amino acid at position 26 is L; and/or
the amino acid at position 27 is L.

In one embodiment it is provided a GIP peptide analogue selected from any one of SEQ ID NOs:1-4, or a functional variant thereof, wherein the amino acid at position 4 is G.

In one embodiment it is provided a GIP peptide analogue selected from any one of SEQ ID NOs:1-4, or a functional variant thereof, wherein the amino acid at position 5 is T.

In one embodiment it is provided a GIP peptide analogue selected from any one of SEQ ID NOs:1-4, or a functional variant thereof, wherein the amino acid at position 6 is F.

In one embodiment it is provided a GIP peptide analogue selected from any one of SEQ ID NOs:1-4, or a functional variant thereof, wherein the amino acid at position 7 is I.

In one embodiment it is provided a GIP peptide analogue selected from any one of SEQ ID NOs:1-4, or a functional variant thereof, wherein the amino acid at position 10 is Y.

In one embodiment it is provided a GIP peptide analogue selected from any one of SEQ ID NOs:1-4, or a functional variant thereof, wherein the amino acid at position 22 is F.

In one embodiment it is provided a GIP peptide analogue selected from any one of SEQ ID NOs:1-4, or a functional variant thereof, wherein the amino acid at position 23 is V.

In one embodiment it is provided a GIP peptide analogue selected from any one of SEQ ID NOs:1-4, or a functional variant thereof, wherein the amino acid at position 25 is W.

In one embodiment it is provided a GIP peptide analogue selected from any one of SEQ ID NOs:1-4, or a functional variant thereof, wherein the amino acid at position 26 is L.

In one embodiment it is provided a GIP peptide analogue selected from any one of SEQ ID NOs:1-4, or a functional variant thereof, wherein the amino acid at position 27 is L.

In one embodiment of the present disclosure there is provided a GIP peptide analogue, as defined herein above, wherein said functional variant has 1 individual amino acid substitution, such as 2 individual amino acid substitutions, for example 3 individual amino acid substitutions, such as 4 individual amino acid substitutions at any amino acid residue of any one of SEQ ID NOs:1-4, or such as 1 to 4 individual amino acid substitutions at any amino acid residue of any one of SEQ ID NOs:1-4.

In one embodiment said functional variant has 1 to 2 individual amino acid substitutions, such as 2 to 3 individual amino acid substitutions, such as 3 to 4 individual amino acid substitutions, such as 4 to 5 individual amino acid substitutions, such as 5 to 6 individual amino acid substitutions, such as 6 to 7 individual amino acid substitutions, such as 7 to 8 individual amino acid substitutions at any amino acid residue of any one of SEQ ID NOs:1-4.

In one embodiment said functional variant has 1 individual amino acid substitution, such as 2 individual amino acid substitutions, for example 3 individual amino acid substitutions, such as 4 individual amino acid substitutions at any amino acid residue of any one of SEQ ID NOs:1-4, wherein said substitutions are conservative amino acid substitutions.

In one embodiment said functional variant has 1 to 7 individual amino acid substitutions, such as 1 individual amino acid substitutions, such as 2 individual amino acid substitutions, such as 3 individual amino acid substitutions, such as 4 individual amino acid substitutions, such as 5 individual amino acid substitutions, such as 6 individual amino acid substitutions, such as 7 individual amino acid substitutions at any one of amino acid residues 3 to 30 of any one of SEQ ID NOs:1-4.

In one embodiment there is provided a GIP peptide analogue as defined herein above, wherein at least one amino acid residue of the GIP peptide analogue of any one of SEQ ID NOs:1-4 is substituted with E, such as wherein at least one amino acid residue at any one of positions 9, 15, and 21 of any one of SEQ ID NOs:1-4 is substituted with E.

Substitution of one or more amino acid residues at any one of positions 9, 15, and 21 of the peptide of any one of SEQ ID NOs:1-4 with E as defined herein may result in increased antagonistic effect, increased solubility, and/or increased stability of the substituted peptide.

In one embodiment there is provided a GIP peptide analogue as defined herein above, wherein X₁ is an amino acid residue selected from the group consisting of E, glutaric acid, succinic acid and adipic acid.

In one embodiment there is provided a GIP peptide analogue as defined herein above, wherein X₁ is E.

In one embodiment there is provided a GIP peptide analogue as defined herein above, wherein X₁ is glutaric acid.

In one embodiment there is provided a GIP peptide analogue according to any one of the preceding claims, wherein X₁ is succinic acid.

In one embodiment there is provided a GIP peptide analogue according to any one of the preceding claims, wherein X₁ is adipic acid.

GIP peptide analogues according to the present disclosure having E at position 3 may be very potent antagonists at the GIPR. However, having E in position 3 may also lead to compounds which are unstable. Without wishing to be bound by theory, E at position 3 may form a pyroGlu by cyclization between the amino group at the N-terminus and the side chain carboxylic acid of E. It may therefore be an advantage to substitute the E at position 3. The present inventors have found that the amino group at the N-terminus may not be necessary for obtaining potent antagonists.

It may be advantageous to substitute E in position 3 (i.e. the first amino acid from the N-terminus) with glutaric acid, since glutaric acid has no amino group and therefore the N-terminal pyroGlu formation is not possible. PyroGlu formation may be an unwanted side reaction for glutamic acid. Substitution with glutaric acid in position 3 may also increase the potency. Glutaric acid is naturally produced in the body during the metabolism of some amino acids, including lysine and tryptophan. Instead of glutaric acid, succinic acid and adipic acid may be used.

In one embodiment there is provided a GIP peptide analogue as defined herein above, wherein the D at position 9 of any one of SEQ ID NOs:1-4, or a functional variant thereof, is substituted with any amino acid, such as substituted with E.

An advantage of having E at position 9 is that the potency and/or stability and/or solubility may be increased.

In one embodiment there is provided a GIP peptide analogue as defined herein above, wherein the S at position 11 of any one of SEQ ID NOs:1-4, or a functional variant thereof, is substituted with any amino acid, such as a conservative amino acid substitution or such as substituted with an amino acid residue selected from the group consisting of A, and K.

In one embodiment there is provided a GIP peptide analogue as defined herein above, wherein the I at position 12 of any one of SEQ ID NOs:1-4, or a functional variant thereof, is substituted with any amino acid, such as a conservative amino acid substitution, or such as substituted with K.

In one embodiment there is provided a GIP peptide analogue as defined herein above, wherein the A at position 13 of SEQ ID NO:1 and SEQ ID NO:2, or a functional variant thereof, is substituted with any amino acid, such as a conservative amino acid substitution, such as substituted with 2-Aminoisobutyric acid (Aib) or K.

In one embodiment there is provided a GIP peptide analogue as defined herein above, wherein the A at position 13 of SEQ ID NO:1 and SEQ ID NO:2, or a functional variant thereof, is substituted with 2-Aminoisobutyric acid (Aib).

An advantage of having Aib at position 13 is that the GIPR antagonistic effect may be considerably increased. In addition, Aib in position 13 may also increase the stability of the peptide, such as the in vivo stability or physical stability.

In one embodiment there is provided a GIP peptide analogue as defined herein above, wherein the M at position 14 of any one of SEQ ID NOs:1-4, or a functional variant thereof, is substituted with any amino acid, such as a conservative amino acid substitution, such as substituted with an amino acid residue selected from the group consisting of L and Norleucine (Nle).

Since M is prone to oxidation it may be an advantage to substitute it with another amino acid such as L or Nle, which may also retain potency.

In one embodiment there is provided a GIP peptide analogue as defined herein above, wherein the D at position 15 of any one of SEQ ID NOs:1-4, or a functional variant thereof, is substituted with any amino acid, such as a conservative amino acid substitution, such as substituted with E. An advantage of having E at position 15 is that the potency and/or stability and/or solubility may be increased.

In one embodiment there is provided a GIP peptide analogue as defined herein above, wherein the K at position 16 of any one of SEQ ID NOs:1-4, or a functional variant thereof, is substituted with any amino acid, such as a conservative amino acid substitution, such as substituted with R.

In one embodiment there is provided a GIP peptide analogue as defined herein above, wherein the I at position 17 of any one of SEQ ID NOs:1-4, or a functional variant thereof, is substituted with any amino acid, such as a conservative amino acid substitution, such as substituted with K.

In one embodiment there is provided a GIP peptide analogue as defined herein above, wherein the H at position 18 of any one of SEQ ID NOs:1-4, or a functional variant thereof, is substituted with any amino acid, such as a conservative amino acid substitution, such as substituted with K.

In one embodiment there is provided a GIP peptide analogue as defined herein above, wherein the Q at position 20 of any one of SEQ ID NOs:1-4, or a functional variant thereof, is substituted with any amino acid, such as a conservative amino acid substitution or such as substituted with K.

In one embodiment there is provided a GIP peptide analogue as defined herein above, wherein the D at position 21 of any one of SEQ ID NOs:1-4, or a functional variant thereof, is substituted with any amino acid, such as a conservative amino acid substitution, such as substituted with E. An advantage of having E at position 21 is that the potency and/or stability and/or solubility may be increased.

In one embodiment there is provided a GIP peptide analogue as defined herein above, wherein the N at position 24 of SEQ ID NO:1 and SEQ ID NO:3, or a functional variant thereof, is substituted with any amino acid, such as a conservative amino acid substitution, such as substituted with Q or such as substituted with E.

In one embodiment the GIP peptide analogue comprises at least one substitution to K and one substitution to E or Aib at any one of amino acid residues 3 to 30 of any one of SEQ ID NOs:1-4.

In one embodiment it is provided a GIP peptide analogue selected from any one of SEQ ID NOs:1-4, wherein:
the amino acid residue at position 3 is E, glutaric acid, succinic acid or adipic acid,
the amino acid residue at position 4 is G,
the amino acid residue at position 5 is T,
the amino acid residue at position 6 is F,
the amino acid residue at position 7 is I,
the amino acid residue at position 8 is S,
the amino acid residue at position 9 is D or E,
the amino acid residue at position 10 is Y,
the amino acid residue at position 11 is S, K or A,
the amino acid residue at position 12 is I or K,
the amino acid residue at position 13 is A, Aib or K,
the amino acid residue at position 14 is M, L or Nle,
the amino acid residue at position 15 is D or E,
the amino acid residue at position 16 is K or R,
the amino acid residue at position 17 is I or K,
the amino acid residue at position 18 is H or K,
the amino acid residue at position 19 is Q,
the amino acid residue at position 20 is Q or K,
the amino acid residue at position 21 is D or E,
the amino acid residue at position 22 is F,
the amino acid residue at position 23 is V,
the amino acid residue at position 24 is N, Q or E,
the amino acid residue at position 25 is W,
the amino acid residue at position 26 is L,
the amino acid residue at position 27 is L,
the amino acid residue at position 28 is A,
the amino acid residue at position 29 is Q, and/or
the amino acid residue at position 30 is K,
or a functional variant thereof.

In one embodiment it is provided a GIP peptide analogue selected from any one of SEQ ID NOs:1-4), wherein
the amino acid residue at position 3 is E, glutaric acid, succinic acid or adipic acid,
the amino acid residue at position 4 is G,
the amino acid residue at position 5 is T,
the amino acid residue at position 6 is F,
the amino acid residue at position 9 is D or E,
the amino acid residue at position 10 is Y,
the amino acid residue at position 11 is S, K or A,
the amino acid residue at position 12 is I or K,
the amino acid residue at position 13 is A, Aib or K,
the amino acid residue at position 14 is M, L or Nle,
the amino acid residue at position 15 is D or E,
the amino acid residue at position 16 is K or R,
the amino acid residue at position 18 is H or K,
the amino acid residue at position 19 is Q,
the amino acid residue at position 20 is Q or K,
the amino acid residue at position 21 is D or E,
the amino acid residue at position 22 is F,
the amino acid residue at position 23 is V,
the amino acid residue at position 24 is N, Q or E,
the amino acid residue at position 25 is W,
the amino acid residue at position 26 is L, and/or
the amino acid residue at position 27 is L, or a functional variant thereof.

In one embodiment it is provided a GIP peptide analogue selected from any one of SEQ ID NOs:1-4, wherein
the amino acid residue at position 3 is E, glutaric acid, succinic acid or adipic acid,
the amino acid residue at position 4 is G,
the amino acid residue at position 5 is T,
the amino acid residue at position 6 is F,
the amino acid residue at position 9 is D or E,
the amino acid residue at position 13 is A, Aib or K,
the amino acid residue at position 14 is M, L or Nle,
the amino acid residue at position 15 is D or E,
the amino acid residue at position 18 is H or K,
the amino acid residue at position 21 is D or E,
the amino acid residue at position 22 is F,
the amino acid residue at position 23 is V,
the amino acid residue at position 24 is N, Q or E,
the amino acid residue at position 25 is W,
the amino acid residue at position 26 is L, and/or
the amino acid residue at position 27 is L,
or a functional variant thereof, wherein said functional variant has 1 individual amino acid substitution, such as 2 individual amino acid substitutions, for example 3 individual amino acid substitutions, such as 4 individual amino acid substitutions at any amino acid residue of any one of SEQ ID NOs: 1-4 or such as 1 to 4 individual amino acid substitutions at any amino acid residue of any one of SEQ ID NOs: 1-4.

In one embodiment there is provided a GIP peptide analogue as defined herein above, wherein Z comprises one or more amino consecutive acid residues of the C-terminus of Exendin-4(31-39) (PSSGAPPPS; SEQ ID NO:67; CE31-39).

In one embodiment there is provided a GIP peptide analogue as defined herein above, wherein Z consists of one or more amino consecutive acid residues of the C-terminus of Exendin-4(31-39) (PSSGAPPPS; SEQ ID NO:67; CE31-39).

In one embodiment there is provided a GIP peptide analogue as defined herein above, wherein Z consists of one or more amino consecutive acid residues of the C-terminus of Exendin-4(30-39) (GPSSGAPPPS; SEQ ID NO:61; CE30-39).

In some embodiments Z comprises at least one G or one P. In some embodiments Z comprises at least two P.

In one embodiment there is provided a GIP peptide analogue as defined herein above,
wherein Z is a peptide selected from the group consisting of
a glycine or a proline,
GP, GPS, GPSS, GPSSG, GPSSGA, GPSSGAP, GPSSGAPP, GPSSGAPPP and GPSSGAPPPS,
PS, PSS, PSSG, PSSGA, PSSGAP, PSSGAPP, PSSGAPPP and PSSGAPPPS,
GPSSGA, GPSSGAP, GPSSGAPP, GPSSGAPPP, GPSSGAPPPS, or a variant thereof comprising 1 or 2 individual amino acid substitutions at any one of the amino acid residues, or
PSSG, PSSGA, PSSGAP, PSSGAPP, PSSGAPPP and PSSGAPPPS, or a variant thereof comprising 1 or 2 individual amino acid substitutions at any one of the amino acid residues.

In one embodiment there is provided a GIP peptide analogue as defined herein above, wherein the fatty acid molecule is not attached at the amino acid residue at position 3 or the N-terminal amino group of the amino acid residue at position 3 of any one of SEQ ID NOs:1-4 or a functional variant thereof.

In one embodiment there is provided a GIP peptide analogue as defined herein above, wherein the GIP peptide analogue has a free N-terminus. Thus, the N-terminus of the GIP peptide analogue comprises an amino (—NH₂) moiety which is not substituted, such as which is not acetylated, acylated or alkylated. Hence, the N-terminus of the GIP peptide analogue may comprise a free amino (—NH₂) moiety.

In one embodiment there is provided a GIP peptide analogue as defined herein above, wherein the fatty acid molecule is attached to the side chain of an amino acid residue at position 11, position 12, position 13, position 16, position 17, position 18, position 20, position 34 if present or position 40 if present of said GIP peptide analogue, such as of any one of SEQ ID NOs:1-4, or a functional variant thereof.

In one embodiment there is provided a GIP peptide analogue as defined herein above, wherein said fatty acid molecule is attached to an amino acid residue at any one of positions 12, 13, 16, 17, 18, position 34 if present or position 40 if present of any one of SEQ ID NOs:1-4, or a functional variant thereof.

In one embodiment there is provided a GIP peptide analogue as defined herein above, wherein said fatty acid molecule is attached to an amino acid residue at position 18 of any one of SEQ ID NOs:1-4, or a functional variant thereof.

In one embodiment there is provided a GIP peptide analogue as defined herein above, wherein a fatty acid molecule is attached to the epsilon-amino group of a K residue of said GIP peptide analogue, such as of any one of SEQ ID NOs:1-4, or a functional variant thereof comprising at least one K residue.

In one embodiment there is provided a GIP peptide analogue as defined herein above, wherein a fatty acid molecule is attached to the side chain amino group of the amino acid residue at position 18 of any one of SEQ ID NOs:1-4, or a functional variant thereof, wherein H at position 18 has been substituted with K or Orn in any one of SEQ ID NOs:1-4.

Attachment of a fatty acid, with or without linker, to the side chain amino group of the amino acid residue at position 18 may result in a GIP peptide analogue with particularly high antagonistic potency.

In one embodiment there is provided a GIP peptide analogue as defined herein above, wherein a fatty acid molecule is attached to the side chain amino group of the amino acid residue at position 11 of any one of SEQ ID NOs:1-4, or a functional variant thereof, wherein S at position 11 has been substituted with K or Orn in any one of SEQ ID NOs:1-4.

In one embodiment there is provided a GIP peptide analogue as defined herein above, wherein a fatty acid molecule is attached to the side chain amino group of the amino acid residue at position 12 of any one of SEQ ID NOs:1-4, or a functional variant thereof, wherein I at position 12 has been substituted with K or Orn in any one of SEQ ID NOs:1-4.

In one embodiment there is provided a GIP peptide analogue as defined herein above, wherein said GIP peptide analogue has an amino acid sequence selected from the group consisting of:

EGTFISEYSAibANleEKIKQQDFVEWLLAQK-Z; SEQ ID NO: 70;
GIP(3-30) [D9E;I12Aib;M14Nle;D15E;H18K;N24E],
EGTFISEYSIAibMEKIKQQDFVEWLLAQK-Z; SEQ ID NO: 72;
GIP(3-30) [D9E;A13Aib;D15E;H18K;N24E],
EGTFISDYSIAMDKIKQQDFVEWLLAQK-Z; SEQ ID NO: 46;
GIP(3-30) [H18K;N24E],
EGTFISDYSIAibMDKIKQQDFVEWLLAQK-Z; SEQ ID NO 73;
GIP(3-30) [A13Aib;H18K;N24E],
EGTFISDYSIAibLDKIKQQDFVEWLLAQK-Z; SEQ ID NO: 74;
GIP(3-30) [A13Aib;M14L;H18K;N24E],
EGTFISDYSIAibNleDKIKQQDFVEWLLAQK-Z; SEQ ID NO: 75;
GIP(3-30) [A13Aib;M14Nle;H18K;N24E],
EGTFISDYSIALDKIKQQDFVEWLLAQK-Z; SEQ ID NO: 76;
GIP(3-30) [M14L;H18K;N24E],
EGTFISDYSIANleDKIKQQDFVEWLLAQK-Z; SEQ ID NO: 77;
GIP(3-30) [M14Nle;H18K;N24E],
EGTFISDYSIAKDKIKQQDFVEWLLAQK-Z; SEQ ID NO: 78;
GIP(3-30) [M14K;H18K;N24E],
EGTFISEYSIAibLEKIKQQEFVEWLLAQK-Z; SEQ ID NO: 79;
GIP(3-30) [D9E;A13Aib;M14L;D15E;H18K;D21E;N24E],
EGTFISEYSIAibNleEKIKQQEFVEWLLAQK-Z; SEQ ID NO: 80;
GIP(3-30) [D9E;A13Aib;M14Nle;D15E;H18K;D21E;N24E],
EGTFISEYSIALEKIKQQEFVEWLLAQK-Z; SEQ ID NO: 81;
GIP(3-30) [D9E;M14L;D15E;H18K;D21E;N24E],
EGTFISEYSIANleEKIKQQEFVEWLLAQK-Z; SEQ ID NO: 82;
GIP(3-30) [D9E;M14Nle;D15E;H18K;D21E;N24E],
XGTFISDYSIANleDKIKQQDFVEWLLAQK-Z; SEQ ID NO: 83;
GIP(3-30) [E3Glutaric acid(X);M14Nle;H18K;N24E],
EGTFISEYSIAibLEKIKQQDFVEWLLAQK-Z; SEQ ID NO: 84;
GIP(3-30) [D9E;A13Aib;M14L;D15E;H18K;N24E]
XGTFISEYSIAibLEKIKQQEFVEWLLAQK-Z; SEQ ID NO: 85;
GIP(3-30) [E3Glutaric acid;D9E;A13Aib;M14L;D15E;
H18K;D21E;N24E],
XGTFISEYSIAibNleEKIKQQEFVEWLLAQK-Z; SEQ ID NO: 86;
GIP(3-30) [E3Glutaric acid;D9E;A13Aib;M14Nle;
D15E;H18K;D21E;N24E],
XGTFISEYSIALEKIKQQEFVEWLLAQK-Z; SEQ ID NO: 87;
GIP(3-30) [E3Glutaric acid;D9E;M14L;D15E;
H18K;D21E;N24E],
XGTFISEYSIAibMEKIKQQEFVEWLLAQK-Z; SEQ ID NO: 88;
GIP(3-30) [E3Glutaric acid;D9E;A13Aib;D15E;H18K;
D21E;N24E],
EGTFISEYSIAibMEKIKQQEFVEWLLAQK-Z; SEQ ID NO: 89;
GIP(3-30) [D9E;A13Aib;D15E;H18K;D21E;N24E],
EGTFISEYSIAMEKIKQQEFVEWLLAQK-Z; SEQ ID NO: 90;
GIP(3-30) [D9E;D15E;H18K;D21E;N24E],
EGTFISEYSIAibLDKIKQQDFVEWLLAQK-Z; SEQ ID NO: 91;
GIP(3-30) [D9E;A13Aib;M14L;H18K;N24E]
XGTFISDYSIAibMDKIKQQDFVEWLLAQK-Z; SEQ ID NO: 92;
GIP(3-30) [E3Glutaric acid;A13Aib;H18K;N24E],
EGTFISDYSKAMDKIHQQDFVEWLLAQK-Z; SEQ ID NO: 93;
GIP(3-30) [I12K;N24E],
EGTFISDYSIKMDKIHQQDFVEWLLAQK-Z; SEQ ID NO: 94;
GIP(3-30) [A13K;N24E],
EGTFISDYSIAMDKIHQQDFVEWLLAQK-Z; SEQ ID NO: 95;
GIP(3-30) [N24E],
EGTFISDYSIAMDKKHQQDFVEWLLAQK-Z; SEQ ID NO: 96;
GIP(3-30) [I17K;N24E],
EGTFISDYSIAMDKIHQQDFVEWLLAQKPSSKAPPPS; SEQ ID
NO: 40; GIP(3-30) [N24E]CEX(31-39)[34K],
EGTFISDYSIAMDKIHQQDFVEWLLAQKPSSGAPPPS; SEQ ID
NO: 41; GIP(3-30) [N24E]CEX(31-39)[40K],
EGTFISDYAIAMDKKHQQDFVEWLLAQK-Z; SEQ ID NO: 97;
GIP(3-30) [S11A;H18K;N24E],
EGTFISEYSIAibMEKIKQQDFVEWLLAQK-Z; SEQ ID NO: 72;
GIP(3-30) [D9E,A13Aib;D15E;H18K;N24E]
XGTFISEYSIAibLEKIKQQEFVEWLLAQK-Z; SEQ ID NO: 98;
GIP(3-30) [E3Succinic acid;D9E;A13Aib;M14L;D15E;
H18K;D21E;N24E],
XGTFISEYSIAibLEKIKQQEFVEWLLAQK-Z; SEQ ID NO: 99;
GIP(3-30) [E3Adipic acid;D9E;A13Aib;M14L;D15E;
H18K;D21E;N24E],
EGTFISDYSIAibMDKIKQQDFVNWLLAQK-Z; SEQ ID NO: 100;
GIP(3-30) [A13Aib;H18K],
XGTFISDYSIAMDKIKQQDFVEWLLAQK-Z; SEQ ID NO: 101;
GIP(3-30) [E3Glutaric acid;H18K;N24E],
and
XGTFISDYSIAibMDKIKQQDFVNWLLAQK-Z; SEQ ID NO: 102;
GIP(3-30) [E3Glutaric acid;A13Aib;H18K],

wherein said peptide is modified by attaching a fatty acid molecule at any position of any one of the above sequences, and wherein said peptide may be C-terminally carboxylated.

In one embodiment the GIP peptide analogue is C-terminally amidated (—NH₂) or C-terminally carboxylated (—COOH).

In one embodiment the GIP peptide analogue is C-terminally carboxylated (—COOH). Without being bound to any theory, a free C-terminal carboxylic acid may be able to assist in increased solubility.

### Functional Variants—Mutants

In one embodiment, one or more, or all, of said amino acid substitutions are conservative amino acid substitutions (or synonymous substitutions). A conservative substitution is the substitution of amino acids whose side chains have similar biochemical properties and thus do not affect the function of the peptide.

Particular amino acid substitutions as disclosed herein are K to R; E to D, glutaric acid; M to L; Q to E; I to V; I to L, Aib; A to Aib; Y to W; S to T; N to S; M to Nle; H to K; D to E; N to Q.

In another embodiment, a functional variant as defined herein includes sequences wherein an alkyl amino acid is substituted for an alkyl amino acid, wherein an aromatic amino acid is substituted for an aromatic amino acid, wherein a sulfur-containing amino acid is substituted for a sulfur-containing amino acid, wherein a hydroxy-containing amino acid is substituted for a hydroxy-containing amino acid, wherein an acidic amino acid is substituted for an acidic amino acid, wherein a basic amino acid is substituted for a basic amino acid, and/or wherein a dibasic monocarboxylic amino acid is substituted for a dibasic monocarboxylic amino acid.

Conservative substitutions may be introduced in any one or more of the above specified positions of a GIP peptide analogue selected from any one of SEQ ID NO:1, SEQ ID NO:2, SEQ ID NO:3 and SEQ ID NO:4, as long as the resulting variant remains functional. It may however also be desirable to introduce non-conservative substitutions in one or more positions (non-synonymous substitutions).

A non-conservative substitution leading to the formation of a variant of a GIP peptide analogue selected from any one of SEQ ID NO:1, SEQ ID NO:2, SEQ ID NO:3 and SEQ ID NO:4 in one embodiment comprises substitution of amino acid residues that i) differ substantially in polarity, for example a residue with a non-polar side chain (Ala, Leu, Pro, Trp, Val, Ile, Leu, Phe or Met) substituted for a residue with a polar side chain such as Gly, Ser, Thr, Cys, Tyr, Asn, or Gln or a charged amino acid such as Asp, Glu, Arg, or Lys, or substituting a charged or a polar residue for a non-polar one; and/or ii) differ substantially in its effect on peptide backbone orientation such as substitution of or for Pro or Gly by another residue; and/or iii) differ substantially in electric charge, for example substitution of a negatively charged residue such as Glu or Asp for a positively charged residue such as Lys, His or Arg (and vice versa); and/or iv) differ substantially in steric bulk, for example substitution of a bulky residue such as His, Trp, Phe or Tyr for one having a minor side chain, e.g. Ala, Gly or Ser (and vice versa).

Substitution of amino acids can in one embodiment be made based upon their hydrophobicity and hydrophilicity values and the relative similarity of the amino acid side-chain substituents, including charge, size, and the like.

The GIP peptide analogues or their functional variant counterparts as defined herein comprise proteinogenic or natural amino acids, i.e. the 22 amino acids naturally incorporated into polypeptides. Of these, 20 are encoded by the universal genetic code and the remaining 2; selenocysteine (Sec, U) and pyrrolysine (Pyl, O), are incorporated into proteins by unique synthetic mechanisms.

A GIP peptide analogue as defined herein in one embodiment comprises one or more non-naturally occurring amino acid residues (unnatural, non-proteinogenic or non-standard amino acids) or amino acid mimetics, such as glutaric acid. Non-naturally occurring amino acids include e.g., without limitation, Aib, beta-2-naphthyl-alanine, trans-3-methylproline, 2,4-methanoproline, cis-4-hydroxyproline, ornithine (Orn), trans-4-hydroxyproline, N-methylglycine, allo-threonine, methylthreonine, hydroxyethylcysteine, hydroxyethylhomocysteine, nitroglutamnine, homoglutamine, pipecolic acid, thiazolidine carboxylic acid, dehydroproline, 3- and 4-methylproline, 3,3-dimethylproline, tert-leucine, norleucine (Nle), methoxinine (Mox), norvaline, 2-azaphenylalanine, 3-azaphenylalanine, 4-azaphenylalanine, and 4-fluorophenylalanine.

In one embodiment the amino acid Met is substituted with an oxidation resistant amino acid analogue, for example, norleucine (Nie) or Leu which preserves the length of the amino acid side chain important for hydrophobic interactions but not its hydrogen-bonding properties; or methoxinine (Mox), a non-canonical amino acid that resembles more closely the electronic properties of Met in comparison to Nie; or Lys.

The standard and/or non-standard amino acids may be linked by peptide bonds (to form a linear peptide chain), or by non-peptide bonds (e.g. via the variable side-chains of the amino acids). Preferably, the amino acids of the peptides defined herein are linked by peptide bonds.

The term peptide also embraces post-translational modifications introduced by chemical or enzyme-catalyzed reactions, as are known in the art. These include acetylation, phosphorylation, methylation, glucosylation, glycation, amidation, hydroxylation, deimination, deamidation, carbamylation and sulfation of one or more amino acid residues, and also proteolytic modification by known proteinases including lysosomal kathepsins, and also calpains, secretases and matrix-metalloproteinases.

Also, functional equivalents of the peptides may comprise chemical modifications such as ubiquitination, labeling (e.g., with radionuclides, various enzymes, etc.), pegylation (derivatization with polyethylene glycol), or by insertion (or substitution by chemical synthesis) of amino acids such as ornithine, which do not normally occur in human proteins (non-proteinogenic).

Sterically similar compounds may be formulated to mimic the key portions of the peptide structure. This may be achieved by techniques of modelling and chemical designing known to those of skill in the art. For example, esterification and other alkylations may be employed to modify the amino terminus of e.g. a di-arginine peptide backbone, to mimic a tetra peptide structure. It will be understood that all such sterically similar constructs fall within the scope of the present invention. Peptides with N-terminal and C-terminal alkylations and esterifications are also encompassed within the present invention. For example, glutaric acid is a sterically similar compound that mimics Glutamic acid.

In one embodiment the N-terminal amino acid of the GIP peptide analogues of the present disclosure does not have any chemical modifications. It may be advantageous that the amino group at the N-terminus of the GIP peptide analogue is free, i.e. not substituted, since substitution may lead to agonistic effects at the GIPR.

In one embodiment the N-terminus, i.e. the NH2 group at the N-terminal, is absent, such as e.g. when position 3 is substituted with glutaric acid, which does not contain an amino group.

It appears that extending the length of the fatty acid or the linker, if present, may decrease the antagonistic potency. However, simultaneously incorporating an Aib residue at position 13 appears to compensate for some or all of the reduced potency, especially in combination with E at one or more of positions 9, 15, 21 and 24, such as in combination with E at position 24.

### Attachment of Fatty Acid Molecules

In one embodiment a fatty acid molecule is attached to one or more amino acid residues having a side-chain amino-alkyl group (—CₙH₂ₙNH₂).

In one embodiment a fatty acid molecule is attached to one or more amino acid residues having a side-chain amino group (NH₂).

In one embodiment a fatty acid molecule is attached to an amino group (NH₂) of an amino acid residue.

In one embodiment a fatty acid molecule is attached to the side-chain amino group of an amino acid residue.

In one embodiment a fatty acid molecule is attached to the E (epsilon) side-chain amino group of a lysine residue (Lys, K).

In one embodiment a fatty acid molecule is attached to the 6 (delta) side-chain amino group of an ornithine residue (Orn).

In one embodiment the amino acid residue having a fatty acid molecule attached is selected from the group consisting of Lys and Orn.

In one embodiment the amino acid residue having a fatty acid molecule attached is Lys.

In one embodiment the fatty acid molecule is attached to the delta-amino group of a Orn residue of said GIP peptide analogue, such as of any one of SEQ ID NOs:1-4, or a functional variant comprising an Orn amino acid residue.

In one embodiment the fatty acid molecule is attached to the epsilon-amino group of a K residue of said GIP peptide analogue, such as of any one of SEQ ID NOs:1-4, or a functional variant thereof.

In one embodiment of the present disclosure there is provided a GIP peptide analogue which is modified by attaching one fatty acid molecule, wherein said fatty acid molecule is a straight-chain fatty acid.

In one embodiment of the present disclosure there is provided a GIP peptide analogue which is modified by attaching one fatty acid molecule, wherein said fatty acid molecule is a branched fatty acid.

In one embodiment of the present disclosure there is provided a GIP peptide analogue which is modified by attaching one fatty acid molecule, wherein said fatty acid molecule is a monoacyl fatty acid molecule, comprising one acyl group. Preferably, the carboxyl group is located at one end of the fatty acid molecule.

For example, a GIP peptide may be conjugated to a monoacyl fatty acid (such as Hexadecanoyl) via a linker, L, as depicted in Formula I:

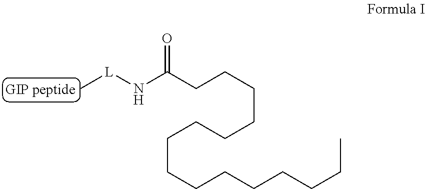

In one embodiment of the present disclosure there is provided a GIP peptide analogue which is modified by attaching one fatty acid molecule, wherein said fatty acid molecule is a diacyl fatty acid molecule. A diacyl fatty acid molecule is a fatty acid molecule comprising two carboxyl groups. Preferably, one or both the carboxyl groups are located at one or each of the endings of the fatty acid molecule.

For example, a GIP peptide may be conjugated to a diacyl fatty, acid also referred to as “diacid”, (such as 15-carboxy-pentadecanoyl) via a linker, L, as depicted in Formula II:

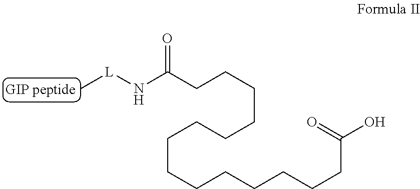

In one embodiment of the present disclosure there is provided a GIP peptide analogue which is modified by attaching one fatty acid molecule, wherein said fatty acid molecule comprises an acyl group of the formula CH₃(CH₂)ₙCO—, wherein n is in an integer from 4 to 24.

In one embodiment said fatty acid molecule comprises one or more acyl groups selected from the group consisting of CH₃(CH₂)₆CO—, CH₃(CH₂)₈CO—, CH₃(CH₂)₁₀CO—, CH₃(CH₂)₁₂CO—, CH₃(CH₂)₁₄CO—, CH₃(CH₂)₁₆CO—, CH₃(CH₂)₁₈CO—, CH₃(CH₂)₂₀CO— and CH₃(CH₂)₂₂CO—.

In one embodiment said fatty acid molecule comprises an acyl group selected from the group consisting of CH₃(CH₂)₁₀CO— (lauryl, C12), CH₃(CH₂)₁₂CO— (myristoyl, C14), CH₃(CH₂)₁₄CO— (palmitoyl, C16), CH₃(CH₂)₁₆CO— (stearyl, C18), CH₃(CH₂)₁₈CO— (arachidyl, C20) and CH₃(CH₂)₂₀CO— (behenyl, C22).

In one embodiment of the present disclosure there is provided a GIP peptide analogue which is modified by attaching one fatty acid molecule, said fatty acid molecule comprises two acyl groups individually selected from the group consisting of HOOC—CH₃(CH₂)₁₀CO— (dodecanoyl, C12), HOOC—CH₃(CH₂)₁₂CO— (1-tetradecanoyl, C14), HOOC—CH₃(CH₂)₁₄CO— (hexadecanoyl, C16), HOOC—CH₃(CH₂)₁₅CO— (15-carboxy-pentadecanoyl, C17), HOOC—CH₃(CH₂)₁₆CO— (octadecanoyl, C18), HOOC—CH₃(CH₂)₁₇CO— (17-carboxy-heptadecanoyl, C19), HOOC—CH₃(CH₂)₁₈CO— (eicosanoyl, C20), HOOC—CH₃(CH₂)₁₉CO— (19-carboxy-nonadecanoyl, C21) and HOOC—CH₃(CH₂)₂₀CO— (behenyl, C22).

In one embodiment, said fatty acid molecule comprises an acyl group of the formula COOH(CH₂)ₙCO— (dicarboxylic acid), wherein n is an integer from 4 to 24.

In one embodiment of the present disclosure there is provided a GIP peptide analogue which is modified by attaching one fatty acid molecule, said fatty acid molecule comprises an acyl group selected from the group consisting of COOH(CH₂)₁₄CO—, COOH(CH₂)₁₆CO—, COOH(CH₂)₁₈CO— and COOH(CH₂)₂₀CO—.

In one embodiment said fatty acid molecule comprises or consists of COOH(CH₂)₁₄CO—

In one embodiment said fatty acid molecule comprises or consists of COOH(CH₂)₁₆CO—

In one embodiment said fatty acid molecule comprises or consists of COOH(CH₂)₁₈CO—

In one embodiment of the present disclosure there is provided a GIP peptide analogue which is modified by attaching one fatty acid molecule, wherein said fatty acid molecule is attached to the epsilon amino group of the side chain of an amino acid residue of said GIP peptide analogue directly.

### Attachment of Fatty Acid Molecules Via a Linker

Attachment of fatty acid molecules to a peptide herein can occur either directly in indirectly, i.e. via a linker or spacer.

In one embodiment of the present disclosure there is provided a GIP peptide analogue which is modified by attaching one fatty acid molecule, wherein said fatty acid molecule is attached to an amino acid residue via a linker.

In one embodiment the fatty acid molecule according to the present disclosure is attached to an amino acid residue via a linker or spacer as depicted in Formula 11:

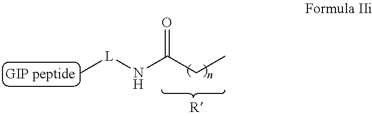

In one embodiment the fatty acid molecule is attached to an amino acid residue via a linker in such a way that a carboxyl group of the fatty acid molecule forms an amide bond with an amino group of the linker.

In some embodiment said linker comprises one or more moieties individually selected from the group consisting of:
a. one or more an α,ω-amino acids,
b. one or more amino acids selected from the group consisting of succinic acid, Lys, Glu, Asp,
c. 4-Abu,
d. y-aminobuturic acid
e. a dipeptide, such as a dipeptide wherein the C-terminal amino acid residue is Lys, His or Trp, preferably Lys, and wherein the N-terminal amino acid residue is selected from the group comprising Ala, Arg, Asp, Asn, Gly, Glu, Gln, lie, Leu, Val, Phe and Pro, such as Gly-Lys,
f. one or more of γ-aminobutanoyl (γ-aminobutyric acid), γ-glutamyl (γ-glutamic acid), β-asparagyl, β-alanyl and glycyl, and
g. γ-glutamic acid—[8-amino-3,6-dioxaoctanoic acid]ₙ (γGlu-AEEAcₙ), wherein n is an integer between 1 and 50, such as an integer between 1-2, 2-3, 3-4, 4-5, 5-6, 6-7, 7-8, 8-9, 9-10, 10-11, 11-12, 12-13, 13-14, 14-15, 15-20, 20-25, 25-30, 30-35, 35-40, 40-45, 45-50.

In some embodiments said linker comprises one or more moieties individually selected from the group consisting of:
a. α-amino acid, γ-amino acid or ω-amino acid,
b. one or more amino acids selected from the group consisting of succinic acid, Lys, Glu, Asp,
c. one or more amino acids selected from the group consisting of Gly and Ser,
d. one or more amino acids selected from the group consisting of Ala, Glu, Lys and Leu,
e. one or more of γ-aminobutanoyl (γ-aminobutyric acid), γ-Glu (γ-glutamic acid), β-Asp (β-asparagyl), β-Ala (β-alanyl), 2-aminoisobutyric acid (Aib) and Gly, and
f. [8-amino-3,6-dioxaoctanoic acid]ₙ (AEEAcₙ), wherein n is an integer between 1 and 50, such as an integer between 1-4, 1-3 or 1-2.

In one embodiment said linker comprises a γ-Glu, one or more 8-amino-3,6-dioxaoctanoic acid (AEEAc), or combinations thereof.

In one embodiment said linker comprises or consists of GGGS or SGGG.

In one embodiment said linker comprises or consists of ALEA or AELA.

In one embodiment said linker comprises or consists of 2-aminoisobutyric acid (Aib).

In one embodiment said linker comprises or consists of yGlu.

In one embodiment said linker comprises or consists of KAAAEKAAAEKAAAE.

In one embodiment of the present disclosure, said linker comprises or consists of a [8-amino-3,6-dioxaoctanoic acid]ₙ (AEEAc)ₙ, wherein n is an integer between 1 and 50, such as an integer between 1-2, 2-3, 3-4, 4-5, 5-6, 6-7, 7-8, 8-9, 9-10, 10-11, 11-12, 12-13, 13-14, 14-15, 15-20, 20-25, 25-30, 30-35, 35-40, 40-45, 45-50, preferably wherein n is 1, 2 or 3.

In one embodiment of the present disclosure, said linker comprises or consists of a γ-Glu and one AEEAc, such as a γ-Glu and two AEEAc, for example a γ-Glu and three AEEAc.

The examples of linkers disclosed herein are such that they can be attached to an amino acid residue of the GIP peptide analogue via any one of the extremities of the linker. Thus, if for example the linker comprises one or more repeats of γ-glutamic acid—8-amino-3,6-dioxaoctanoic acid (γ-Glu)-(AEEAcₙ), said linker can be attached to an amino acid residue of the GIP peptide analogue either via a γ-Glu or via a AEEAcₙ.

In one embodiment the linker is [γ-glutamic acid]—[8-amino-3,6-dioxaoctanoic acid](γ-Glu)-AEEAc or [8-amino-3,6-dioxaoctanoic acid]—[γ-glutamic acid](AEEAc-γ-Glu). For example, a GIP peptide may be conjugated to a fatty acid (for example C16 or palmitic acid/palmitoyl in Formula IV, but any other fatty acid may be used) via [γ-glutamic acid]—[8-amino-3,6-dioxaoctanoic acid] as depicted in Formula IV:

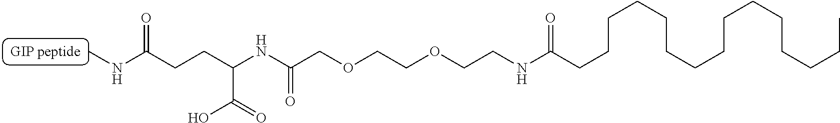 [SMILES: CCCCCCCCCCCCCCCC(=O)NCCOCCOCC(=O)NC(CCC(=O)NC)C(=O)O]

Formula IV: the formula does not depict the stereochemistry, usually, the natural L-form is used, unless otherwise specified.

For example, a GIP peptide may be conjugated to a fatty acid (for example C16 or palmitic acid/palmitoyl in Formula IV, but any other fatty acid may be used) via [8-amino-3,6-dioxaoctanoic acid]—[γ-glutamic acid] as depicted in Formula V:

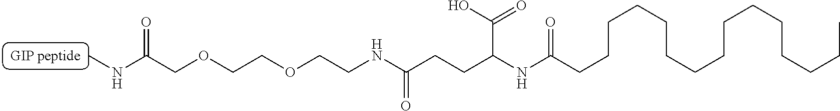 [SMILES: CCCCCCCCCCCCCCCC(=O)NC(CCC(=O)NCCOCCOCC(=O)NC)C(=O)O]

Formula V: the formula does not depict the stereochemistry, usually, the natural L-form is used, unless otherwise specified.

In one embodiment of the present disclosure, the fatty acid molecule is attached to an amino acid residue via a linker, and wherein the combination of linker and fatty acid is selected from the group consisting of:
i. Hexadecanoyl-γ-Glu-
ii. Hexadecanoyl-γ-Glu-γ-Glu-
iii. Hexadecanoyl-γ-Glu-AEEAc-
iv. Hexadecanoyl-γ-Glu-AEEAc-AEEAc-
v. Hexadecanoyl-γ-Glu-AEEAc-AEEAc-AEEAc-
vi. [15-carboxy-pentadecanoyl]-γ-Glu-
vii. [15-carboxy-pentadecanoyl]-γ-Glu-γ-Glu-
viii. [15-carboxy-pentadecanoyl]-γ-Glu-AEEAc-
ix. [15-carboxy-pentadecanoyl]-γ-Glu-AEEAc-AEEAc-
x. [15-carboxy-pentadecanoyl]-γ-Glu-AEEAc-AEEAc-AEEAc-
xi. Octadecanoyl-γ-Glu-
xii. Octadecanoyl-γ-Glu-γ-Glu-
xiii. Octadecanoyl-γ-Glu-AEEAc-
xiv. Octadecanoyl-γ-Glu-AEEAc-AEEAc-
xv. Octadecanoyl-γ-Glu-AEEAc-AEEAc-AEEAc-
xvi. [17-carboxy-heptadecanoyl]-γ-Glu-
xvii. [17-carboxy-heptadecanoyl]-γ-Glu-γ-Glu-
xviii. [17-carboxy-heptadecanoyl]-γ-Glu-AEEAc-
xix. [17-carboxy-heptadecanoyl]-γ-Glu-AEEAc-AEEAc-
xx. [17-carboxy-heptadecanoyl]-γ-Glu-AEEAc-AEEAc-AEEAc-
xxi. Eicosanoyl-γ-Glu-
xxii. Eicosanoyl-γ-Glu-γ-Glu-
xxiii. Eicosanoyl-γ-Glu-AEEAc-
xxiv. Eicosanoyl-γ-Glu-AEEAc-AEEAc-
xxv. Eicosanoyl-γ-Glu-AEEAc-AEEAc-AEEAc-
xxvi. [19-carboxy-nonadecanoyl]-γ-Glu-
xxvii. [19-carboxy-nonadecanoyl]-γ-Glu-γ-Glu-
xxviii. [19-carboxy-nonadecanoyl]-γ-Glu-AEEAc-
xxix. [19-carboxy-nonadecanoyl]-γ-Glu-AEEAc-AEEAc-
xxx. [19-carboxy-nonadecanoyl]-γ-Glu-AEEAc-AEEAc-AEEAc-.

In one embodiment of the present disclosure, the fatty acid molecule is attached to an amino acid residue via a linker, and wherein the combination of linker and fatty acid is selected from the group consisting of:
i. [15-Carboxy pentadecanoyl]-yGlu
ii. [17-carboxy-heptadecanoyl]-γ-Glu-AEEAc-AEEAc-, and
iii. [17-carboxy-heptadecanoyl]-yGlu-yGlu.
   GIP Peptides with Fatty Acid

In one embodiment of the present disclosure there is provided a GIP peptide analogue as defined herein, wherein the GIP peptide analogue is selected from the group consisting of:

EGTFISEYSIAMEKIKQQDFVEWLLAQKPSSGAPPPS-C16-diacid/
18K; SEQ ID NO: 10; GIP(3-30) + Cex(31-39)[D9E;
D15E;H18K;N24E],
EGTFISEYSAibANleEKIKQQDFVEWLLAQKPSSGAPPPS-C16-
diacid/18K; SEQ ID NO: 71; GIP(3-30) + Cex(31-39)
[D9E;I12Aib;M14Nle;D15E;H18K;N24E],
EGTFISEYSIAibMEKIKQQDFVEWLLAQKPSSGAPPPS-C16-
diacid/18K; SEQ ID NO: 33; GIP(3-30) + Cex(31-39)
[D9E;A13Aib;D15E;H18K;N24E],
EGTFISDYSIAMDKIKQQDFVEWLLAQKPSSGAPPPS-C16-diacid/
18K; SEQ ID NO: 47; GIP(3-30) + Cex(31-39)[H18K;
N24E],
EGTFISDYSIAibMDKIKQQDFVEWLLAQKPSSGAPPPS-C16-
diacid/18K; SEQ ID NO: 12; GIP(3-30) + Cex(31-39)
[A13Aib;H18K;N24E],
EGTFISDYSIAibLDKIKQQDFVEWLLAQKPSSGAPPPS-C16-
diacid/18K; SEQ ID NO: 13; GIP(3-30) + Cex(31-39)
[A13Aib;M14L;H18K;N24E],
EGTFISDYSIAibLDKIKQQDFVEWLLAQKPSSGAPPPS-2xAEEAc +
yGlu-C18-diacid/18K; SEQ ID NO: 13; GIP(3-30) +
Cex(31-39)[A13Aib;M14L;H18K;N24E],
EGTFISDYSIAibNleDKIKQQDFVEWLLAQKPSSGAPPPS-C16-
diacid/18K; SEQ ID NO: 14; GIP(3-30) + Cex(31-39)
[A13Aib;M14Nle;H18K;N24E],
EGTFISDYSIAibNleDKIKQQDFVEWLLAQKPSSGAPPPS-
2xAEEAc + yGlu-C18-diacid/18K; SEQ ID NO: 14;
GIP(3-30) + Cex(31-39)[A13Aib;M14Nle;H18K;N24E],
EGTFISDYSIALDKIKQQDFVEWLLAQKPSSGAPPPS-C16-diacid/
18K; SEQ ID NO: 15; GIP(3-30) + Cex(31-39)[M14L;
H18K;N24E],
EGTFISDYSIALDKIKQQDFVEWLLAQKPSSGAPPPS-2xAEEAc +
yGlu-C18-diacid/18K; SEQ ID NO: 15; GIP(3-30) +
Cex(31-39)[M14L;H18K;N24E],
EGTFISDYSIANleDKIKQQDFVEWLLAQKPSSGAPPPS-C16-
diacid/18K; SEQ ID NO: 16; GIP(3-30) + Cex(31-39)
[M14Nle;H18K;N24E],
EGTFISDYSIANleDKIKQQDFVEWLLAQKPSSGAPPPS-2xAEEAc +
yGlu-C18-diacid/18K; SEQ ID NO: 16; GIP(3-30) +
Cex(31-39)[M14Nle;H18K;N24E],
EGTFISDYSIAKDKIKQQDFVEWLLAQKPSSGAPPPS-C16-diacid/
18K; SEQ ID NO: 17; GIP(3-30) + Cex(31-39)[M14K;
H18K;N24E],
EGTFISEYSIAibLEKIKQQEFVEWLLAQKPSSGAPPPS-C16-
diacid/18K; SEQ ID NO: 18; GIP(3-30) + Cex(31-39)
[D9E;A13Aib;M14L;D15E;H18K;D21E;N24E],
EGTFISEYSIAibLEKIKQQEFVEWLLAQKPSSGAPPPS-2xAEEAc +
yGlu-C18-diacid/18K; SEQ ID NO: 18; GIP(3-30) +
Cex(31-39)[D9E;A13Aib;M14L;D15E;H18K;D21E;N24E],
EGTFISEYSIAibNleEKIKQQEFVEWLLAQKPSSGAPPPS-C16-
diacid/18K; SEQ ID NO: 19; GIP(3-30) + Cex(31-39)
[D9E;A13Aib;M14Nle;D15E;H18K;D21E;N24E],
EGTFISEYSIAibNleEKIKQQEFVEWLLAQKPSSGAPPPS-
2xAEEAc + yGlu-C18-diacid/18K; SEQ ID NO: 19;
GIP(3-30) + Cex(31-39)[D9E;A13Aib;M14Nle;D15E;
H18K;D21E;N24E],
XGTFISDYSIAMDKIKQQDFVNWLLAQKPSSGAPPPS-C16-diacid/
18K; SEQ ID NO: 20; GIP(3-30) + Cex(31-39)
[E3Glutaric acid(X);H18K],
EGTFISEYSIALEKIKQQEFVEWLLAQKPSSGAPPPS-2xAEEAc +
yGlu-C18-diacid/18K; SEQ ID NO: 21; GIP(3-30) +
Cex(31-39)[D9E;M14L;D15E;H18K;D21E;N24E],
EGTFISEYSIANleEKIKQQEFVEWLLAQKPSSGAPPPS-2xAEEAc +
yGlu-C18-diacid/18K; SEQ ID NO: 22; GIP(3-30) +
Cex(31-39)[D9E;M14Nle;D15E;H18K;D21EN24E],
XGTFISDYSIANleDKIKQQDFVEWLLAQKPSSGAPPPS-C16-
diacid/18K; SEQ ID NO: 24; GIP(3-30) + Cex(31-39)
[E3Glutaric acid(X);M14Nle;H18K;N24E],
XGTFISDYSIANleDKIKQQDFVEWLLAQKPSSGAPPPS-2xAEEAc +
yGlu-C18-diacid/18K; SEQ ID NO: 24; GIP(3-30) +
Cex(31-39)[E3Glutaricacid(X);M14Nle;H18K;N24E],
XGTFISEYSIAibLEKIKQQEFVEWLLAQKPSSGAPPPS-2xAEEAc +
yGlu-C18-diacid/18K; SEQ ID NO: 25; GIP(3-30) +
Cex(31-39)[E3Glutaricacid;D9E;A13Aib;M14L;D15E;
H18K;D21E;N24E],
XGTFISEYSIAibNleEKIKQQEFVEWLLAQKPSSGAPPPS-
2xAEEAc + yGlu-C18-diacid/18K; SEQ ID NO: 26;
GIP(3-30) − Cex(31-39)[E3Glutaric acid;D9E;A13Aib;
M14Nle;D15E;H18K;D21E;N24E],
XGTFISEYSIALEKIKQQEFVEWLLAQKPSSGAPPPS-OH-2xAEEAc +
yGlu-C18-diacid/18K; SEQ ID NO: 27; GIP(3-30)Cex
(31-39)[E3Glutaric acid;D9E;M14L;D15E;H18K;
D21E;N24E],
XGTFISEYSIAibMEKIKQQEFVEWLLAQKPSSGAPPPS-2xAEEAc +
yGlu-C18-diacid/18K; SEQ ID NO: 28; GIP(3-30 −
Cex(31-39)[E3Glutaric acid;D9E;A13Aib;D15E;H18K;
D21E;N24E],
EGTFISEYSIAibMEKIKQQEFVEWLLAQKPSSGAPPPS-2xAEEAc +
yGlu-C18-diacid/18K; SEQ ID NO: 29; GIP(3-30) −
Cex(31-39)[D9E;A13Aib;D15E;H18K;D21E;N24E],
EGTFISEYSIAMEKIKQQEFVEWLLAQKPSSGAPPPS-2xAEEAc +
yGlu-C18-diacid/18K; SEQ ID NO: 9; GIP(3-30)
Cex(31-39)[D9E;D15E;H18K;D21E;N24E],
EGTFISEYSIAibLDKIKQQDFVEWLLAQKPSSGAPPPS-2xAEEAc +
yGlu-C18-diacid/18K; SEQ ID NO: 30; GIP(3-30)
Cex(31-39)[D9E;A13Aib;M14L;H18K;N24E],
XGTFISEYSIAibLEKIKQQEFVEWLLAQKPSSGAPPPS-2xAEEAc +
yGlu-C18-diacid/18K; SEQ ID NO: 25; GIP(3-30)
Cex(31-39)[E3Glutaric acid;D9E;A13Aib;M14L;
D15E;H18K;D21E;N24E],
EGTFISEYSIAibLEKIKQQEFVEWLLAQKPSSGAPPPS-2xAEEAc +
yGlu-C18-diacid/18K; SEQ ID NO: 18; GIP(3-30)
Cex(31-39)[D9E;A13Aib;M14L;D15E;H18K;D21E;N24E],
XGTFISDYSIAibMDKIKQQDFVEWLLAQKPSSGAPPPS-C16-
diacid/18K; SEQ ID NO: 32; GIP(3-30)Cex(31-39)
[E3Glutaric acid;A13Aib;H18K;N24E],
EGTFISEYSIAibMEKIKQQDFVEWLLAQKPSSGAPPPS-C16-
diacid/18K; SEQ ID NO: 33; GIP(3-30)Cex(31-39)
[D9E;A13Aib;D15E;H18K;N24E],
XGTFISEYSIAibLEKIKQQEFVEWLLAQKPSSGAPPPS-C16-
diacid/18K; SEQ ID NO: 25; GIP(3-30)Cex(31-39)
[E3Glutaric acid;D9E;A13Aib;M14L;D15E;H18K;
D21E;N24E],
EGTFISEYSIALEKIKQQEFVEWLLAQKPSSGAPPPS-C16-diacid/
18K; SEQ ID NO: 21; GIP(3-30)Cex(31-39)
[D9E;M14L;D15E;H18K;N24E],
EGTFISEYSIAibLEKIKQQEFVEWLLAQKPSSGAPPPS-(GGGS-C16-
diacid/18K); SEQ ID NO: 18; GIP(3-30)Cex(31-39)
[D9E;A13Aib;M14L;D15E;H18K;D21E;N24E],
EGTFISEYSIAibLEKIKQQEFVEWLLAQKPSSGAPPPS-(ALEA-C16-
diacid/18K); SEQ ID NO: 18; GIP(3-30)Cex(31-39)
[D9E;A13Aib;M14L;D15E;H18K;D21E;N24E],
EGTFISDYSKAMDKIHQQDFVEWLLAQKPSSGAPPPS-C16-diacid/
12K; SEQ ID NO: 36; GIP(3-30)Cex(31-39)
[I12K;N24E],
EGTFISDYSIKMDKIHQQDFVEWLLAQKPSSGAPPPS-C16-diacid/
13K; SEQ ID NO: 37; GIP(3-30)Cex(31-39)
[A13K;N24E],
EGTFISDYSIAMDKIHQQDFVEWLLAQKPSSGAPPPS-C16-diacid/
16K; SEQ ID NO: 38; GIP(3-30)Cex(31-39)[N24E],
EGTFISDYSIAMDKKHQQDFVEWLLAQKPSSGAPPPS-C16-diacid/
17K; SEQ ID NO: 39; GIP(3-30)Cex(31-39)
[I17K;N24E],
EGTFISDYSIAMDKIHQQDFVEWLLAQKPSSKAPPPS C16-diacid/
34K; SEQ ID NO: 40; GIP(3-30)Cex(31-39)[N24E;34K],
EGTFISDYSIAMDKIHQQDFVEWLLAQKPSSGAPPPSK-C16-diacid/
40K; SEQ ID NO: 41; GIP(3-30)Cex(31-39)[N24E;40K],
EGTFISDYAIAMDKIKQQDFVEWLLAQKPSSGAPPPS-C16-diacid/
18K; SEQ ID NO: 43; GIP(3-30)Cex(31-39)[S11A;
H18K;N24E],
EGTFISDYSIAMDKIKQQDFVEWLLAQK-C16-diacid/18K; SEQ
ID NO: 46; GIP(3-30)[H18K;N24E],
EGTFISDYSIAMDKIKQQDFVEWLLAQKPSSGAPPPS-C20-diacid/
18K; SEQ ID NO: 47; GIP(3-30)Cex(31-39)-OH[H18K;
N24E],
EGTFISDYSIAMDKIKQQDFVEWLLAQKPSSGAPPPS-C16/18K;
SEQ ID NO: 47; GIP(3-30)Cex(31-39)[H18K;N24E],
EGTFISEYSIAibLEKIKQQDFVEWLLAQKPSSGAPPPS-2xAEEAc +
yGlu-C18-diacid/18K; SEQ ID NO: 50; GIP(3-30)
Cex(31-39)[D9E;A13Aib;M14L;D15E;H18K;N24E],
XGTFISEYSIAibNleEKIKQQEFVEWLLAQKPSSGAPPPS-C16-
diacid/18K); SEQ ID NO: 26; GIP(3-30)Cex(31-39)
[E3Glutaric acid;D9E;A13Aib;M14Nle;D15E;H18K;
D21E;N24E],
EGTFISEYSIAibLEKIKQQEFVEWLLAQKPSSGAPPPS-(Aib-C16-
diacid/18K); SEQ ID NO: 18; GIP(3-30)Cex(31-39)
[D9E;A13Aib;M14L;D15E;H18K;D21E;N24E],
EGTFISEYSIAibLEKIKQQEFVEWLLAQKPSSGAPPPS-
(KAAAEKAAAEKAAAE-C16-diacid/18K); SEQ ID NO: 18;
GIP(3-30)Cex(31-39)[D9E;A13Aib;M14L;D15E;H18K;
D21E;N24E],
XGTFISEYSIAibLEKIKQQEFVEWLLAQKPSSGAPPPS-2xAEEAc +
yGlu-C18-diacid/18K; SEQ ID NO: 48; GIP(3-30)
Cex(31-39)[E3Succinic acid;D9E;A13Aib;M14L;D15E;
H18K;D21E;N24E],
XGTFISEYSIAibLEKIKQQEFVEWLLAQKPSSGAPPPS-2xAEEAc +
yGlu-C18-diacid/18K; SEQ ID NO: 49; GIP(3-30)
Cex(31-39)[E3Adipic acid;D9E;A13Aib;M14L;D15E;
H18K;D21E;N24E],
EGTFISDYSIAibMDKIKQQDFVNWLLAQKPSSGAPPPS-C16-
diacid/18K; SEQ ID NO: 52; GIP(3-30)Cex(31-39)
[A13Aib;H18K],
XGTFISDYSIAMDKIKQQDFVEWLLAQKPSSGAPPPS-C16-
diacid/18K; SEQ ID NO: 53; GIP(3-30)Cex(31-39)
[E3Glutaric acid;H18K;N24E],
and
XGTFISDYSIAibMDKIKQQDFVNWLLAQKPSSGAPPPS-C16-
diacid/18K; SEQ ID NO: 54; GIP(3-30)Cex(31-39)
[E3Glutaric acid;A13Aib;H18K],

or a functional variant thereof,
wherein said fatty acid is attached directly or via a linker as defined herein.

It follows that C16 is the fatty acid CH₃(CH₂)₁₄CO— (palmitoyl) and C18 is the fatty acid CH₃(CH₂)₁₆CO— (stearyl). The suffix “-diacid” means that the fatty acid molecule is a diacyl fatty acid molecule. No such suffix refers to a monoacyl fatty acid molecule.

It follows that C20 is the fatty acid CH₃(CH₂)₁₈CO— (arachidyl). The suffix “-diacid” means that the fatty acid molecule is a diacyl fatty acid molecule. No such suffix refers to a monoacyl fatty acid molecule.

It follows that C22 is the fatty acid CH₃(CH₂)₂₀CO— (behenyl). The suffix “-diacid” means that the fatty acid molecule is a diacyl fatty acid molecule. No such suffix refers to a monoacyl fatty acid molecule.

### Determining Antagonist Properties and Affinity

In order to determine whether a peptide is an antagonist of the GIPR, methods known in the art may be employed, for example by determining the IC50 of the peptide. This can be done by constructing a dose-response curve and examining the effect of different concentrations of the peptide on reversing agonist activity. The agonist can be GIP1-42, for example hGIP-1-42 or hGIP1-30. The GIPR can be hGIPR, rGIPR, mGIPR, dog GIPR, pig GIPR or the Macaca mulatta GIPR. IC50 values can be calculated for a given antagonist by determining the concentration needed to inhibit half of the maximum biological response of the agonist. A method for determining whether a peptide is an antagonist is described in example 4, but other methods known in the art may also be used. For example, Schild plot analysis may be performed on hGIP1-42 cAMP dose-response curves with increasing concentrations of GIP-derived peptides. In this way, the type of antagonist activity may also be determined.

The GIP peptide analogues of the present disclosure are characterized by having antagonistic activity towards GIPR. In particular, the GIP peptide analogues of the present disclosure are potent antagonists of GIPR.

In one embodiment of the present disclosure, the GIP peptide analogue inhibits GIPR activity of at least 80%, such as of at least 85%, such as of at least 90%, such as of at least 95%, such as of about 100%, such as measured via an assay that determines the decrease in intracellular cAMP, such as measured via a CisBio cAMP assay (alternative 1) and/or via a “Gaddum” assay (alternative 2), which are described in “Materials and methods”.

In one embodiment of the present disclosure, the GIP peptide analogue inhibits GIPR activity of at least 80%, such as of at least 85%, such as of at least 90%, such as of at least 95%, such as of about 100%, wherein inhibition of GIPR activity is determined as a decrease in intracellular cAMP, such as measured via an assay that determines the decrease in intracellular cAMP, such as via a CisBio cAMP assay (alternative 1) and/or via a “Gaddum” assay (alternative 2), which are described in “Materials and methods”. The % inhibition is a % of inhibition of Emax, which means that if a peptide inhibits Emax of 85%, there is 15% activity left of the GIPR.

In one embodiment of the present disclosure, the GIP peptide analogue has a GIPR antagonistic potency corresponding to an IC50 value of less than 50 nM, such as an IC50 value of less than 10 nM, such as an IC50 value of less than 5 nM, such as an IC50 value of less than 1 nM, such as an IC50 value of between 0.001 nM to 1 nM, wherein antagonistic activity (also referred to as “potency”) is measured via an assay that determines the decrease in intracellular cAMP, such as via a CisBio cAMP assay and/or via a “gaddum” assay, which are described in “Materials and methods”.

Methods for determining antagonistic activity of a compound, such as of a GIP peptide analogue, are known to the person of skills in the art. Exemplary methods that can be used for determining antagonistic activity of a compound, such as of a GIP peptide analogue, can be found herein in the “Examples”, for example, these methods comprise measuring intracellular cAMP and determining a decrease in intracellular cAMP resulting from treatment of cells with a GIP peptide analogue.

The GIP peptide analogues of the present disclosure are also characterized by having low or no agonistic activity towards GIPR. GIP peptide analogues having low or no agonistic activity towards GIPR, such as an agonistic activity of 20% or less, preferably of 10% or less, ever more preferably of 5% or less, are also referred to as “silent antagonists”.

In one embodiment the GIP peptide analogue of the present disclosure is capable of stimulating GIPR activity of at most 30%, such as of at most 25%, such as of at the most 20%, such as of at the most 15%, such as of at the most 10%, such as of at the most 5%, in one embodiment the GIP peptide analogue of the present disclosure has no agonistic activity towards GIPR, that is it stimulates GIPR activity of about 0%.

Agonistic activity of a GIP peptide analogue towards GIPR can be determined in the same way as antagonistic activity, but an increase in intracellular cAMP is measured, instead of a decrease, as described in “Materials and methods”.

### Method of Treatment

It is also an aspect to provide a GIP peptide analogue as defined herein, or a composition comprising the GIP peptide analogue, for use as a medicament.

In one embodiment there is provided a glucose-dependent insulinotropic peptide (GIP) analogue consisting of amino acid sequence SEQ ID NO:1:

3 - 4 - 5 - 6 7 8 9 10 11 12 13
X₁ - G - T - F - I - S - D - Y - S - I - A -
14 15 16 17 18 19 20 21 22 23 24
M - D - K - I H - Q - Q - D - F - V - N -
25 26 27 28 29 30
W - L - L - A - Q - K - Z,

wherein X₁ is any amino acid or omitted;
or a functional variant thereof, wherein said variant has 1 to 8 individual amino acid substitutions at any amino acid of SEQ ID NO:1,
wherein N at position 24 of SEQ ID NO:1, or a functional variant thereof, is substituted with E and/or wherein A at position 13 of SEQ ID NO:1, or a functional variant thereof, is substituted with 2-Aminoisobutyric acid (Aib),
wherein Z is a peptide comprising one or more amino acid residues of the C-terminus of Exendin-4(31-39) (PSSGAPPPS; SEQ ID NO:67; CE31-39) or is omitted, and
wherein said peptide is modified by attaching one fatty acid molecule at one amino acid residue at any position of SEQ ID NO 1 or said functional variant thereof or at one amino acid residue at any position of Z SEQ ID NO:67; CE31-39 for use as a medicament.

In one embodiment there is provided a GIP analogue selected from the group consisting of:

```
(SEQ ID NO: 2)
  3 - 4 - 5 - 6 7 8 9 10 11 12 13
X₁ - G - T - F - I - S - D - Y - S - I - A -
14 15 16 17 18 19 20 21 22 23 24
M - D - K - I H - Q - Q - D - F - V - E -
25 26 27 28 29 30
W - L - L - A - Q - K - Z,,
(SEQ ID NO: 3)
  3 - 4 - 5 - 6 7 8 9 10 11 12 13
X₁ - G - T - F - I - S - D - Y - S - I -Aib -
14 15 16 17 18 19 20 21 22 23 24
M - D - K - I H - Q - Q - D - F - V - N -
25 26 27 28 29 30
W - L - L - A - Q - K - Z,,
and
(SEQ ID NO: 4)
  3 - 4 - 5 - 6 7 8 9 10 11 12 13
X₁ - G - T - F - I - S - D - Y - S - I -Aib -
14 15 16 17 18 19 20 21 22 23 24
M - D - K - I H - Q - Q - D - F - V - E -
25 26 27 28 29 30
W - L - L - A - Q - K - Z,,
```

wherein X₁ is any amino acid or omitted;
or a functional variant thereof, wherein said variant has 1 to 8 individual amino acid substitutions at any amino acid of SEQ ID NO:2 (except E at position 24); of SEQ ID NO:3 (except Aib at position 13); and of SEQ ID NO:4 (except Aib at position 13 and E at position 24);
wherein Z is a peptide comprising one or more amino acid residues of the C-terminus of Exendin-4(31-39) (PSSGAPPPS; SEQ ID NO:67; CE31-39), and
wherein said peptide is modified by attaching one fatty acid molecule at one amino acid residue at any position of SEQ ID NO:2-4, or said functional variant thereof, or at one amino acid residue at any position of Z SEQ ID NO:67; CE31-39 for use as a medicament.

In one embodiment there is provided a glucose-dependent insulinotropic peptide (GIP) analogue consisting of amino acid sequence SEQ ID NO:1:

3 - 4 - 5 - 6 7 8 9 10 11 12 13
X₁ - G - T - F - I - S - D - Y - S - I - A -
14 15 16 17 18 19 20 21 22 23 24
M - D - K - I H - Q - Q - D - F - V - N -
25 26 27 28 29 30
W - L - L - A - Q - K - Z,

wherein X₁ is any amino acid or omitted;
or a functional variant thereof, wherein said variant has 1 to 8 individual amino acid substitutions at any amino acid of SEQ ID NO:1,
wherein N at position 24 of SEQ ID NO:1, or a functional variant thereof, is substituted with E and/or wherein A at position 13 of SEQ ID NO:1, or a functional variant thereof, is substituted with 2-Aminoisobutyric acid (Aib),
wherein Z is a peptide comprising one or more amino acid residues of the C-terminus of Exendin-4(31-39) (PSSGAPPPS; SEQ ID NO:67; CE31-39) or is omitted, and
wherein said peptide is modified by attaching one fatty acid molecule at one amino acid residue at any position of SEQ ID NO 1 or said functional variant thereof or at one amino acid residue at any position of Z SEQ ID NO:67; CE31-39 for use in a method of inhibiting or reducing one or more of i) GIP-induced glucagon secretion, ii) GIP-induced insulin secretion, iii) GIP-induced somatostatin secretion, iv) GIP-induced glucose uptake, v) GIP-induced fatty acid synthesis and/or fatty acid incorporation, vi) high or increased expression or activity of a GIPR, vii) post-prandial GIP release, viii) serum levels of free fatty acids and/or triglycerides, ix) GIP-induced appetite increases, x) GIP-induced reduction in energy expenditure, xi) GIP-induced increase in absorption of nutrients from the gut, xii) GIP-induced decrease in GLP-1's appetite suppressive effect, xiii) GIP-induced leptin resistance.

In one embodiment there is provided a GIP analogue selected from the group consisting of:

```
(SEQ ID NO: 2)
  3 - 4 - 5 - 6 7 8 9 10 11 12 13
X₁ - G - T - F - I - S - D - Y - S - I - A -
14 15 16 17 18 19 20 21 22 23 24
M - D - K - I H - Q - Q - D - F - V - E -
25 26 27 28 29 30
W - L - L - A - Q - K - Z,,
(SEQ ID NO: 3)
  3 - 4 - 5 - 6 7 8 9 10 11 12 13
X₁ - G - T - F - I - S - D - Y - S - I -Aib -
14 15 16 17 18 19 20 21 22 23 24
M - D - K - I H - Q - Q - D - F - V - N -
25 26 27 28 29 30
W - L - L - A - Q - K - Z,,
and
(SEQ ID NO: 4)
  3 - 4 - 5 - 6 7 8 9 10 11 12 13
X₁ - G - T - F - I - S - D - Y - S - I -Aib -
14 15 16 17 18 19 20 21 22 23 24
M - D - K - I H - Q - Q - D - F - V - E -
25 26 27 28 29 30
W - L - L - A - Q - K - Z,,
```

wherein X₁ is any amino acid or omitted;
or a functional variant thereof, wherein said variant has 1 to 8 individual amino acid substitutions at any amino acid of SEQ ID NO:2 (except E at position 24); of SEQ ID NO:3 (except Aib at position 13); and of SEQ ID NO:4 (except Aib at position 13 and E at position 24);
wherein Z is a peptide comprising one or more amino acid residues of the C-terminus of Exendin-4(31-39) (PSSGAPPPS; SEQ ID NO:67; CE31-39), and
wherein said peptide is modified by attaching one fatty acid molecule at one amino acid residue at any position of SEQ ID NO:2-4, or said functional variant thereof, or at one amino acid residue at any position of Z SEQ ID NO:67; CE31-39 for use in a method of inhibiting or reducing one or more of i) GIP-induced glucagon secretion, ii) GIP-induced insulin secretion, iii) GIP-induced somatostatin secretion, iv) GIP-induced glucose uptake, v) GIP-induced fatty acid synthesis and/or fatty acid incorporation, vi) high or increased expression or activity of a GIPR, vii) post-prandial GIP release, viii) serum levels of free fatty acids and/or triglycerides, ix) GIP-induced appetite increases, x) GIP-induced reduction in energy expenditure, xi) GIP-induced increase in absorption of nutrients from the gut, xii) GIP-induced decrease in GLP-1's appetite suppressive effect, xiii) GIP-induced leptin resistance.

In one embodiment there is provided a glucose-dependent insulinotropic peptide (GIP) analogue consisting of amino acid sequence SEQ ID NO:1:

3 - 4 - 5 - 6 7 8 9 10 11 12 13
X₁ - G - T - F - I - S - D - Y - S - I - A -
14 15 16 17 18 19 20 21 22 23 24
M - D - K - I H - Q - Q - D - F - V - N -
25 26 27 28 29 30
W - L - L - A - Q - K - Z,

wherein X₁ is any amino acid or omitted;
or a functional variant thereof, wherein said variant has 1 to 8 individual amino acid substitutions at any amino acid of SEQ ID NO:1,
wherein N at position 24 of SEQ ID NO:1, or a functional variant thereof, is substituted with E and/or wherein A at position 13 of SEQ ID NO:1, or a functional variant thereof, is substituted with 2-Aminoisobutyric acid (Aib),
wherein Z is a peptide comprising one or more amino acid residues of the C-terminus of Exendin-4(31-39) (PSSGAPPPS; SEQ ID NO:67; CE31-39) or is omitted, and
wherein said peptide is modified by attaching one fatty acid molecule at one amino acid residue at any position of SEQ ID NO 1 or said functional variant thereof or at one amino acid residue at any position of Z SEQ ID NO:67; CE31-39 for use in a method of treating a condition selected from the group consisting of metabolic syndrome, obesity, pre-diabetes, type I diabetes, type 2 diabetes, insulin resistance, elevated fasting glucose, hyperglycemia, elevated fasting serum triglyceride levels, low levels of very low-density lipoprotein (VLDL), low high-density lipoprotein (HDL) levels, dyslipidemia, increased/decreased low-density lipoprotein (LDL), high cholesterol levels, abnormal deposition of lipids, a cardiovascular disease, elevated blood pressure and atherosclerosis.

In one embodiment there is provided a GIP analogue selected from the group consisting of:

```
(SEQ ID NO: 2)
  3 - 4 - 5 - 6 7 8 9 10 11 12 13
X₁ - G - T - F - I - S - D - Y - S - I - A -
14 15 16 17 18 19 20 21 22 23 24 25
M - D - K - I H - Q - Q - D - F - V - E - W -
26 27 28 29 30
L - L - A - Q - K - Z,,
(SEQ ID NO: 3)
  3 - 4 - 5 - 6 7 8 9 10 11 12 13
X₁ - G - T - F - I - S - D - Y - S - I -Aib -
14 15 16 17 18 19 20 21 22 23 24
M - D - K - I H - Q - Q - D - F - V - N -
25 26 27 28 29 30
W - L - L - A - Q - K - Z,,
and
(SEQ ID NO: 4)
  3 - 4 - 5 - 6 7 8 9 10 11 12 13
X₁ - G - T - F - I - S - D - Y - S - I -Aib -
14 15 16 17 18 19 20 21 22 23 24
M - D - K - I H - Q - Q - D - F - V - E -
25 26 27 28 29 30
W - L - L - A - Q - K - Z,,
```

wherein X₁ is any amino acid or omitted;
or a functional variant thereof, wherein said variant has 1 to 8 individual amino acid substitutions at any amino acid of SEQ ID NO:2 (except E at position 24); of SEQ ID NO:3 (except Aib at position 13); and of SEQ ID NO:4 (except Aib at position 13 and E at position 24);
wherein Z is a peptide comprising one or more amino acid residues of the C-terminus of Exendin-4(31-39) (PSSGAPPPS; SEQ ID NO:67; CE31-39), and
wherein said peptide is modified by attaching one fatty acid molecule at one amino acid residue at any position of SEQ ID NO:2-4, or said functional variant thereof, or at one amino acid residue at any position of Z SEQ ID NO:67; CE31-39 for use in a method of treating a condition selected from the group consisting of metabolic syndrome, obesity, pre-diabetes, type I diabetes, type 2 diabetes, insulin resistance, elevated fasting glucose, hyperglycemia, elevated fasting serum triglyceride levels, low levels of very low-density lipoprotein (VLDL), low high-density lipoprotein (HDL) levels, dyslipidemia, increased/decreased low-density lipoprotein (LDL), high cholesterol levels, abnormal deposition of lipids, a cardiovascular disease, elevated blood pressure and atherosclerosis.

In one embodiment there is provided a glucose-dependent insulinotropic peptide (GIP) analogue consisting of amino acid sequence SEQ ID NO:1:

3 - 4 - 5 - 6 7 8 9 10 11 12 13
X₁ - G - T - F - I - S - D - Y - S - I - A -
14 15 16 17 18 19 20 21 22 23 24
M - D - K - I H - Q - Q - D - F - V - N -
25 26 27 28 29 30
W - L - L - A - Q - K - Z,

wherein X₁ is any amino acid or omitted;
or a functional variant thereof, wherein said variant has 1 to 8 individual amino acid substitutions at any amino acid of SEQ ID NO:1,
wherein N at position 24 of SEQ ID NO:1, or a functional variant thereof, is substituted with E and/or wherein A at position 13 of SEQ ID NO:1, or a functional variant thereof, is substituted with 2-Aminoisobutyric acid (Aib),
wherein Z is a peptide comprising one or more amino acid residues of the C-terminus of Exendin-4(31-39) (PSSGAPPPS; SEQ ID NO:67; CE31-39) or is omitted, and
wherein said peptide is modified by attaching one fatty acid molecule at one amino acid residue at any position of SEQ ID NO 1 or said functional variant thereof or at one amino acid residue at any position of Z SEQ ID NO:67; CE31-39 for use in the manufacture of a medicament for
   treating a condition selected from the group consisting of metabolic syndrome, obesity, pre-diabetes, type I diabetes, type 2 diabetes, insulin resistance, elevated fasting glucose, hyperglycemia, elevated fasting serum triglyceride levels, low levels of very low-density lipoprotein (VLDL), low high-density lipoprotein (HDL) levels, dyslipidemia, increased/decreased low-density lipoprotein (LDL), high cholesterol levels, abnormal deposition of lipids, a cardiovascular disease, elevated blood pressure and atherosclerosis, or
   inducing weight-loss.

In one embodiment there is provided a GIP analogue selected from the group consisting of:

```
(SEQ ID NO: 2)
  3 - 4 - 5 - 6 7 8 9 10 11 12 13
X₁ - G - T - F - I - S - D - Y - S - I - A -
14 15 16 17 18 19 20 21 22 23 24
M - D - K - I H - Q - Q - D - F - V - E -
25 26 27 28 29 30
W - L - L - A - Q - K - Z,,
(SEQ ID NO: 3)
  3 - 4 - 5 - 6 7 8 9 10 11 12 13
X₁ - G - T - F - I - S - D - Y - S - I -Aib -
14 15 16 17 18 19 20 21 22 23 24
M - D - K - I H - Q - Q - D - F - V - N -
25 26 27 28 29 30
W - L - L - A - Q - K - Z,,
and
(SEQ ID NO: 4)
  3 - 4 - 5 - 6 7 8 9 10 11 12 13
X₁ - G - T - F - I - S - D - Y - S - I -Aib -
14 15 16 17 18 19 20 21 22 23 24
M - D - K - I H - Q - Q - D - F - V - E -
25 26 27 28 29 30
W - L - L - A - Q - K - Z,,
```

wherein X₁ is any amino acid or omitted;
or a functional variant thereof, wherein said variant has 1 to 8 individual amino acid substitutions at any amino acid of SEQ ID NO:2 (except E at position 24); of SEQ ID NO:3 (except Aib at position 13); and of SEQ ID NO:4 (except Aib at position 13 and E at position 24);
wherein Z is a peptide comprising one or more amino acid residues of the C-terminus of Exendin-4(31-39) (PSSGAPPPS; SEQ ID NO:67; CE31-39), and
wherein said peptide is modified by attaching one fatty acid molecule at one amino acid residue at any position of SEQ ID NO:2-4, or said functional variant thereof, or at one amino acid residue at any position of Z SEQ ID NO:67; CE31-39 for use in the manufacture of a medicament for
   treating a condition selected from the group consisting of metabolic syndrome, obesity, pre-diabetes, type I diabetes, type 2 diabetes, insulin resistance, elevated fasting glucose, hyperglycemia, elevated fasting serum triglyceride levels, low levels of very low-density lipoprotein (VLDL), low high-density lipoprotein (HDL) levels, dyslipidemia, increased/decreased low-density lipoprotein (LDL), high cholesterol levels, abnormal deposition of lipids, a cardiovascular disease, elevated blood pressure and atherosclerosis, or
   inducing weight loss.

In one particular embodiment there is provided a GIP peptide analogue as defined herein for use in a method of treating obesity.

In one particular embodiment there is provided a GIP peptide analogue as defined herein for use in a method of treating diabetes mellitus, including diabetes mellitus type I and type II.

In one particular embodiment there is provided a GIP peptide analogue as defined herein for use in a method of treating insulin resistance.

An obesity related disorders may be any one of: increased food-intake, increased appetite, binge eating, bulimia nervosa, obesity induced by administration of an antipsychotic or a steroid, reduced/increased gastric motility, delayed/increased gastric emptying, decreased physical mobility, osteoarthritis, dyslipidemia, increased/decreased low-density lipoprotein (LDL), high cholesterol levels, and abnormal deposition of lipids.

In some embodiments, dyslipidemia, increased/decreased low-density lipoprotein (LDL), cholesterol, and abnormal deposition of lipids are referred to as fatty acid metabolism disorders.

A diabetes related disorders may be any one of: impaired glucose tolerance (IGT), progression from IGT to type 2 diabetes, progression of non-insulin requiring type 2 diabetes to insulin requiring type 2 diabetes, decreased beta-cell function, decreased beta-cell mass, increased beta-cell apoptosis, decreased glucose sensitivity to beta-cells.

A cardiovascular disease may be any one of coronary heart disease, myocardial infarction, reperfusion injury, stroke, cerebral ischemia, left ventricular hypertrophy, coronary artery disease, hypertension, essential hypertension, acute hypertensive emergency, cardiomyopathy, heart insufficiency, exercise intolerance, acute and/or chronic heart failure, arrhythmia, cardiac dysrhythmia, syncopy, angina pectoris, cardiac bypass and/or stent reocclusion, intermittent claudication (also referred to as atherosclerosis oblitterens), diastolic dysfunction, and systolic dysfunction, and combinations thereof.

Also provided is a method for treating metabolic syndrome, obesity, over-weight, diabetes mellitus, insulin resistance, an obesity related disorder as defined herein, or a diabetes related disorder as defined herein; said method comprising the step of administering to an individual in need thereof an effective amount of a peptide as defined herein.

An individual in need as referred to herein, is an individual that may benefit from the administration of a peptide or pharmaceutical composition according to the present disclosure. Such an individual may suffer from metabolic syndrome, and/or from a metabolic disorder such as obesity, over-weight, diabetes, insulin resistance, an obesity related disorder as defined herein, or a diabetes related disorder as defined herein or be in risk of suffering therefrom. The individual may be any human being, male or female, infant, middle-aged or old. The disorder to be treated or prevented in the individual may relate to the age of the individual, the general health of the individual, the medications used for treating the individual and whether or not the individual has a prior history of suffering from diseases or disorders that may have or have induced metabolic syndrome, and/or a metabolic disorder such as obesity, over-weight, diabetes, insulin resistance, an obesity related disorder as defined herein, or a diabetes related disorder as defined herein. In some embodiments, the disorder to be treated is linked to GIP-induced glucagon secretion, GIP-induced insulin secretion, to GIP-induced somatostatin secretion, to GIP-induced glucose uptake, to GIP-induced fatty acid synthesis and/or fatty acid incorporation, to high expression and/or activity of a GIPR, to release of GIP following a meal; wherein the term “high” is to be construed as referring to levels greater than the corresponding levels observed in individuals not in need of treatment.

Method of Preparation (peptide)

The peptides according to the present disclosure may be prepared by any methods known in the art. Thus, the GIP-derived peptides may be prepared by standard peptide-preparation techniques such as solution synthesis or Merrifield-type solid phase synthesis.

In one embodiment, a peptide as defined herein is a non-naturally occurring peptide; being derived from naturally occurring native GIP, such as GIP(1-42).

In one embodiment a peptide according to the disclosure is synthetically made or produced.

The methods for synthetic production of peptides are well known in the art. Detailed descriptions as well as practical advice for producing synthetic peptides may be found in Synthetic Peptides: A User's Guide (Advances in Molecular Biology), Grant G. A. ed., Oxford University Press, 2002, or in: Pharmaceutical Formulation: Development of Peptides and Proteins, Frokjaer and Hovgaard eds., Taylor and Francis, 1999.

In one embodiment the peptide or peptide sequences of the invention are produced synthetically, in particular, by the Sequence Assisted Peptide Synthesis (SAPS) method, by solution synthesis, by Solid-phase peptide synthesis (SPPS) such as Merrifield-type solid phase synthesis, by recombinant techniques (production by host cells comprising a first nucleic acid sequence encoding the peptide operably associated with a second nucleic acid capable of directing expression in said host cells) or enzymatic synthesis. These are well-known to the skilled person.

Peptides may be synthesised either batch-wise on a fully automated peptide synthesiser using 9-fluorenylmethyloxycarbonyl (Fmoc) or tert-Butyloxycarbonyl (Boc) as N-a-amino protecting group and suitable common protection groups for side-chain functionalities.

After purification such as by reversed phase HPLC, peptides may be further processed to obtain for example cyclic or C- or N-terminal modified isoforms. The methods for cyclization and terminal modification are well-known in the art.

Peptides according to the invention may be synthesized as monomers or multimers such as dimers or tetramers.

### Pharmaceutical Composition and Formulation

Whilst it is possible for the bioactive agent of the present disclosure to be administered as the raw chemical (peptide), it is sometimes preferred to present them in the form of a pharmaceutical formulation. Such a pharmaceutical formulation may be referred to as a pharmaceutical composition, pharmaceutically acceptable composition or pharmaceutically safe composition.

Accordingly, further provided is a pharmaceutical formulation, which comprises a bioactive agent of the present invention, or a pharmaceutically acceptable salt or ester thereof, and a pharmaceutically acceptable carrier, excipient and/or diluent. The pharmaceutical formulations may be prepared by conventional techniques, e.g. as described in Remington: The Science and Practice of Pharmacy 2005, Lippincott, Williams & Wilkins.

Pharmaceutically acceptable salts of the instant peptide compounds, where they can be prepared, are also intended to be covered by this invention. These salts will be ones which are acceptable in their application to a pharmaceutical use. By that it is meant that the salt will retain the biological activity of the parent compound and the salt will not have untoward or deleterious effects in its application and use in treating diseases.

Pharmaceutically acceptable salts are prepared in a standard manner. If the parent compound is a base it may for example be treated with an excess of an organic or inorganic acid in a suitable solvent. If the parent compound is an acid, it may for example be treated with an inorganic or organic base in a suitable solvent.

The peptide compounds as disclosed herein may be administered in the form of an alkali metal or earth alkali metal salt thereof, concurrently, simultaneously, or together with a pharmaceutically acceptable carrier or diluent, especially and preferably in the form of a pharmaceutical composition thereof, whether by oral, rectal, or parenteral (including subcutaneous) route, in an effective amount.

Examples of pharmaceutically acceptable acid addition salts for use in the present inventive pharmaceutical composition include those derived from mineral acids, such as hydrochloric, hydrobromic, phosphoric, metaphosphoric, nitric and sulfuric acids, and organic acids, such as tartaric, acetic, citric, malic, lactic, fumaric, benzoic, glycolic, gluconic, succinic, p-toluenesulphonic acids, and arylsulphonic, for example.

In a particular embodiment, the peptide according to the disclosure is formulated as an acetate salt, a Cl− (chloride) salt or Na+ (sodium) salt.

In certain embodiments of the present invention (e.g. liquid compositions) the composition is stable, such as physical stable, for at least 4 days of usage. In further embodiments the composition is stable for at least 2 weeks of usage and for at least 6 months of storage. In still further embodiments, the composition is stable for at least 2 weeks of usage and at least one year of storage. In even further embodiments the composition is stable for at least 4 weeks of usage and for at least 2 years of storage. In this regard, the term “usage” for the purposes of this paragraph refers to taking the pharmaceutical composition out of storage for the purpose of employing the composition for therapeutic purposes, and thereby subjecting it to ambient conditions (conditions of light, dark, temperature, agitation etc.), whilst the term “storage” for the purposes of this paragraph refers to storage under non-agitated conditions in a refrigerator or freezer at a temperature not exceeding about 5 degrees Celsius. The skilled worker will understand the typical range of usage and storage conditions that these pharmaceutical compositions may be subjected to.

### Administration and Dosage

According to the present disclosure, a peptide, or a composition comprising a peptide as defined herein is administered to individuals in need of treatment in pharmaceutically effective doses or a therapeutically effective amount. The dosage requirements will vary with the particular drug composition employed, the route of administration and the particular subject being treated, which depend on the severity and the sort of the disorder as well as on the weight and general state of the subject. It will also be recognized by one skilled in the art that the optimal quantity and spacing of individual dosages of a peptide compound will be determined by the nature and extent of the condition being treated, the form, route and site of administration, and the particular patient being treated, and that such optima can be determined by conventional techniques. It will also be appreciated by one of skill in the art that the optimal course of treatment, i.e., the number of doses of a compound given per day for a defined number of days, can be ascertained using conventional course of treatment determination tests.

In one embodiment the bioactive agent is administered at least once daily, such as once daily, such as twice daily, such as thrice daily, such as four times daily, such as five times daily.

A dose may also be administered in intermittent intervals, or intervals, whereby a dose is not administered every day. Rather one or more doses may be administered every second day, every third day, every fourth day, every fifth day, every sixth day, every week, every second week, every third week, every fourth week, every fifth week, every sixth week, or intervals within those ranges (such as every 2 to 4 weeks, or 4 to 6 weeks).

In one embodiment, a dose is administered once every week, such as once weekly, such as in one dose per week.

### Routes of Administration

It will be appreciated that the preferred route of administration will depend on the general condition and age of the subject to be treated, the nature of the condition to be treated, the location of the tissue to be treated in the body and the active ingredient chosen, but may for example be subcutaneous.

### Systemic Treatment

For systemic treatment according to the present disclosure the route of administration is capable of introducing the bioactive agent into the blood stream to ultimately target the sites of desired action.

Such routes of administration are any suitable routes, such as an enteral route (including the oral, rectal, nasal, pulmonary, buccal, sublingual, transdermal, intracisternal and intraperitoneal administration), and/or a parenteral route (including subcutaneous, intramuscular, intrathecal, intracerebral, intravenous and intradermal administration).

### Parenteral Administration

Parenteral administration is any administration route not being the oral/enteral route whereby the medicament avoids first-pass degradation in the liver. Accordingly, parenteral administration includes any injections and infusions, for example bolus injection or continuous infusion, such as intravenous administration, intramuscular administration or subcutaneous administration. Furthermore, parenteral administration includes inhalations and topical administration.

Accordingly, the bioactive agent may be administered topically to cross any mucosal membrane of an animal to which the biologically active substance is to be given, e.g. in the nose, vagina, eye, mouth, genital tract, lungs, gastrointestinal tract, or rectum, preferably the mucosa of the nose, or mouth, and accordingly, parenteral administration may also include buccal, sublingual, nasal, rectal, vaginal and intraperitoneal administration as well as pulmonal and bronchial administration by inhalation or installation. Also, the agent may be administered topically to cross the skin.

According to an advantageous embodiment of the invention, the GIP analogue is administered subcutaneously.

### Local Treatment

The bioactive agent according to the invention may in one embodiment be used as a local treatment, i.e. be introduced directly to the site(s) of action. Accordingly, the bioactive agent may be applied to the skin or mucosa directly, or the bioactive agent may be injected into the site of action, for example into the diseased tissue or to an end artery leading directly to the diseased tissue. These administration forms preferably avoid the blood brain barrier.

### Kit-of-Parts

The present disclosure also relates to a kit-of-parts comprising one or more of the bioactive agents described above and at least one additional or further component, such as one or more second active ingredients.

### REFERENCES

1. Baggio L L, Drucker D J. Biology of Incretins: GLP-1 and GIP. Gastroenterology 2007; 132(6):2131-2157.
2. Holst J J. On the Physiology of GIP and GLP-1. Horm Metab Res 2004; 36(11/12):747-754.
3. Heer J, Rasmussen C, Coy D H, Holst J J. Glucagon-like peptide-1, but not glucose-dependent insulinotropic peptide, inhibits glucagon secretion via somatostatin (receptor subtype 2) in the perfused rat pancreas. Diabetologia 2008; 51(12):2263-2270.
4. Gutniak M, Orskov C, Holst J J, Ahren B, Efendic S. Antidiabetogenic Effect of Glucagon-like Peptide-1 (7-36)amide in Normal Subjects and Patients with Diabetes Mellitus. N Engl J Med 1992; 326(20):1316-1322.
5. Christensen M, Vedtofte L, Holst J J, Vilsboell T, Knop F K. Glucose-Dependent Insulinotropic Polypeptide: A Bifunctional Glucose-Dependent Regulator of Glucagon and Insulin Secretion in Humans. Diabetes 2011; 60(12):3103-3109.
6. Pederson R, Brown J. Interaction of Gastric Inhibitory Polypeptide, Glucose, and Arginine on Insulin and Glucagon Secretion from the Perfused Rat Pancreas. Endocrinology 1978; 103(2):610-615.
7. Adrian T E, Bloom S R, Hermansen K, Iversen J. Pancreatic polypeptide, glucagon and insulin secretion from the isolated perfused canine pancreas. Diabetologia 1978; 14(6):413-417.
8. Brunicardi F C, Druck P, Seymour N E, Sun Y S, Elahi D, Andersen D K. Selective neurohormonal interactions in islet cell secretion in the isolated perfused human pancreas. Journal of Surgical Research 1990; 48(4):273-278.
9. Dupre J, Caussignac Y, McDonald T J, Van Vliet S. Stimulation of Glucagon Secretion by Gastric Inhibitory Polypeptide in Patients with Hepatic Cirrhosis and Hyperglucagonemia. The Journal of Clinical Endocrinology & Metabolism 1991; 72(1):125-129.
10. Ding W G, Renstrom E, Rorsman P, Buschard K, Gromada J. Glucagon-like peptide I and glucose-dependent insulinotropic polypeptide stimulate Ca2+-induced secretion in rat alpha-cells by a protein kinase A-mediated mechanism. Diabetes 1997; 46(5):792-800.
11. Meier J J, Gallwitz B, Siepmann N et al. Gastric inhibitory polypeptide (GIP) dose-dependently stimulates glucagon secretion in healthy human subjects at euglycaemia. Diabetologia 2003; 46(6):798-801.
12. Christensen M B, Calanna S, Holst J J, Vilsboell T, Knop F K. Glucose-dependent Insulinotropic Polypeptide: Blood Glucose Stabilizing Effects in Patients With Type 2 Diabetes. The Journal of Clinical Endocrinology & Metabolism 2013; 99(3):E418-E426.
13. Christensen M, Calanna S, Sparre-Ulrich A H et al. Glucose-Dependent Insulinotropic Polypeptide Augments Glucagon Responses to Hypoglycemia in Type 1 Diabetes. Diabetes 2014.
14. Song D H, Getty-Kaushik L, Tseng E, Simon J, Corkey B E, Wolfe M M. Glucose-Dependent Insulinotropic Polypeptide Enhances Adipocyte Development and Glucose Uptake in Part Through Akt Activation. Gastroenterology 2007; 133(6):1796-1805.
15. Miyawaki K, Yamada Y, Ban N et al. Inhibition of gastric inhibitory polypeptide signaling prevents obesity. Nat Med 2002; 8(7):738-742.
16. Starich G H, Bar R S, Mazzaferri E L. GIP increases insulin receptor affinity and cellular sensitivity in adipocytes. Am J Physiol 1985; 249(6 Pt 1):E603-E607.
17. Getty-Kaushik L, Song D H, Boylan M O, Corkey B E, Wolfe M M. Glucose-Dependent Insulinotropic Polypeptide Modulates Adipocyte Lipolysis and Reesterification. Obesity 2006; 14(7):1124-1131.
18. Hauner H, Glatting G, Kaminska D, Pfeiffer E F. Effects of gastric inhibitory polypeptide on glucose and lipid metabolism of isolated rat adipocytes. Ann Nutr Metab 1988; 32(5-6):282-288.
19. Kim S J, Nian C, Karunakaran S, Clee S M, Isales C M, McIntosh CHS. GIP-Overexpressing Mice Demonstrate Reduced Diet-Induced Obesity and Steatosis, and Improved Glucose Homeostasis. PLoS ONE 2012; 7(7):e40156.
20. Nasteska D, Harada N, Suzuki K et al. Chronic Reduction of GIP Secretion Alleviates Obesity and Insulin Resistance Under High-Fat Diet Conditions. Diabetes 2014; 63(7):2332-2343.
21. Miyawaki K, Yamada Y, Yano H et al. Glucose intolerance caused by a defect in the entero-insular axis: A study in gastric inhibitory polypeptide receptor knockout mice. Proceedings of the National Academy of Sciences 1999; 96(26):14843-14847.
22. Ahlqvist E, Osmark P, Kuulasmaa T et al. Link Between GIP and Osteopontin in Adipose Tissue and Insulin Resistance. Diabetes 2013; 62(6):2088-2094.
23. Calanna S, Christensen M, Holst J J et al. Secretion of Glucose-Dependent Insulinotropic Polypeptide in Patients With Type 2 Diabetes: Systematic review and meta-analysis of clinical studies. Diabetes Care 2013; 36(10):3346-3352.
24. Asmar M, Simonsen L, Madsbad S, Stallknecht B, Holst J J, Bulow J. Glucose-Dependent Insulinotropic Polypeptide May Enhance Fatty Acid Re-esterification in Subcutaneous Abdominal Adipose Tissue in Lean Humans. Diabetes 2010; 59(9):2160-2163.
25. Deschamps I, Heptner W, Desjeux J F, Baltakse V, Machinot S, Lestradet H. Effects of diet on insulin and gastric inhibitory polypeptide levels in obese children. Pediatr Res 1980; 14(4 Pt 1):300-303.
26. Brøns C, Jensen C B, Storgaard H et al. Impact of short-term high-fat feeding on glucose and insulin metabolism in young healthy men. The Journal of Physiology 2009; 587(10):2387-2397.
27. Raufman J P, Singh L, Eng J. Exendin-3, a novel peptide from Heloderma horridum venom, interacts with vasoactive intestinal peptide receptors and a newly described receptor on dispersed acini from guinea pig pancreas. Description of exendin-3(9-39) amide, a specific exendin receptor antagonist. Journal of Biological Chemistry 1991; 266(5):2897-2902.
28. Jorgensen N B, Dirksen C, Bojsen-Moller K N et al. Exaggerated Glucagon-Like Peptide 1 Response Is Important for Improved β-Cell Function and Glucose Tolerance After Roux-en-Y Gastric Bypass in Patients With Type 2 Diabetes. Diabetes 2013; 62(9):3044-3052.
29. Nakamura T, Tanimoto H, Mizuno Y, Tsubamoto Y, Noda H. Biological and functional characteristics of a novel lowGçômolecular weight antagonist of glucose-dependent insulinotropic polypeptide receptor, SKL-14959, in vitro and in vivo. Diabetes, Obesity and Metabolism 2012; 14(6):511-517.
30. Ebert R, Illmer K, Creutzfeldt W. Release of gastric inhibitory polypeptide (GIP) by intraduodenal acidification in rats and humans and abolishment of the incretin effect of acid by GIP-antiserum in rats. Gastroenterology 1979; 76(3):515-523.
31. Fulurija A, Lutz T A, Sladko K et al. Vaccination against GIP for the Treatment of Obesity. PLoS ONE 2008; 3(9):e3163.
32. Irwin N, McClean P L, Patterson S, Hunter K, Flatt P R. Active immunisation against gastric inhibitory polypeptide (GIP) improves blood glucose control in an animal model of obesity-diabetes. Biological Chemistry. bchm 390, 75. 2009. 16-7-2014.
33. Hinke S A, Manhart S, Pamir N et al. Identification of a bioactive domain in the amino-terminus of glucose-dependent insulinotropic polypeptide (GIP). Biochimica et Biophysica Acta (BBA)—Protein Structure and Molecular Enzymology 2001; 1547(1):143-155.
34. Tseng C C, Kieffer T J, Jarboe L A, Usdin T B, Wolfe M M. Postprandial stimulation of insulin release by glucose-dependent insulinotropic polypeptide (GIP). Effect of a specific glucose-dependent insulinotropic polypeptide receptor antagonist in the rat. J Clin Invest 1996; 98(11):2440-2445.
35. Irwin N, Green B D, Parker J C, Gault V A, O'Harte FPM, Flatt P R. Biological activity and antidiabetic potential of synthetic fragment peptides of glucose-dependent insulinotropic polypeptide, GIP(1-16) and (Pro3)GIP(1-16). Regulatory Peptides 2006; 135(1GQ62):45-53.
36. Kerr B D, Flatt AJS, Flatt P R, Gault V A. Characterization and biological actions of N-terminal truncated forms of glucose-dependent insulinotropic polypeptide. Biochemical and Biophysical Research Communications 2011; 404(3):870-876.
37. Gelling R W, Coy D H, Pederson R A et al. GIP(6-30amide) contains the high affinity binding region of GIP and is a potent inhibitor of GIP1-42 action in vitro. Regulatory Peptides 1997; 69(3):151-154.
38. Deacon CFP. GIP-(3-42) does not antagonize insulinotropic effects of GIP at physiological concentrations. American Journal of Physiology—Endocrinology and Metabolism 2006; 291(3):E468-E475.
39. Gault V A, O'Harte FPM, Harriott P, Flatt P R. Characterization of the Cellular and Metabolic Effects of a Novel Enzyme-Resistant Antagonist of Glucose-Dependent Insulinotropic Polypeptide. Biochemical and Biophysical Research Communications 2002; 290(5):1420-1426.
40. Ravn P, Madhurantakam C, Kunze S et al. Structural and Pharmacological Characterization of Novel Potent and Selective Monoclonal Antibody Antagonists of Glucose-dependent Insulinotropic Polypeptide Receptor. Journal of Biological Chemistry 2013; 288(27):19760-19772.
41. Deacon C F, Plamboeck A, Rosenkilde M M, de Heer J, Holst J J. GIP-(3-42) does not antagonize insulinotropic effects of GIP at physiological concentrations. American Journal of Physiology—Endocrinology and Metabolism 2006; 291(3):E468-E475.
42. Goetze J P, Hunter I, Lippert S K, Bardram L, Rehfeld J F. Processing-independent analysis of peptide hormones and prohormones in plasma. Front Biosci 2012; 17:1804-1815.
43. Goetze J P, Rehfeld J F. Peptide hormones and their prohormones as biomarkers. Biomarkers Med 2009; 3(4):335-338.
44. Fujita Y, Asadi A, Yang G K, Kwok Y N, Kieffer T J. Differential processing of pro-glucose-dependent insulinotropic polypeptide in gut. American Journal of Physiology—Gastrointestinal and Liver Physiology 2010; 298(5):G608-G614.
45. Widenmaier S B, Kim S J, Yang G K et al. A GIP Receptor Agonist Exhibits beta-Cell Anti-Apoptotic Actions in Rat Models of Diabetes Resulting in Improved beta-Cell Function and Glycemic Control. PLoS ONE 2010; 5(3):e9590.
46. Graham F L, van der Eb A J. A new technique for the assay of infectivity of human adenovirus 5 DNA. Virology 1973; 52(2):456-467.
47. Kissow H, Hartmann B, Holst J J et al. Glucagon-like peptide-1 (GLP-1) receptor agonism or DPP-4 inhibition does not accelerate neoplasia in carcinogen treated mice. Regulatory Peptides 2012; 179(1-3):91-100.

### EXAMPLES

The present examples support the following conclusions:
1) The GIP peptide analogues according to embodiments of the present disclosure comprising substitutions A13Aib and/or N24E have increased solubility and/or stability, such as physical stability.
2) Individual amino acid substitutions at certain sites, such as Aib at position 13, result in improved antagonistic effect at the GIP receptor.
3) Several acylation sites show great potential on GIP(3-30)+Z with substitutions A13Aib and/or N24E, such as e.g. position 18.

### Materials and Methods

The generation and action of GIP(3-30) peptides per se is disclosed in WO 2016/034186.

### Materials

Human GIP(1-42) was purchased from Phoenix Pharmaceuticals Inc. while the remaining GIP peptide analogues were synthesized by Caslo™, Lyngby, Denmark and Almac Group, Craigavon, United Kingdom, Peptides & Elephants GmbH, Henningsdorf, Germany, and WuXi AppTec, China. cDNA of the human GIP receptor was purchased from Origene, Rockville, Maryland, USA (SC110906) and cloned into a pCMV-Script vector.

### Transfections and Cell CULTURE

COS-7 cells were cultured at 10% CO₂ and 37° C. in Dulbecco's modified Eagle's medium 1885 supplemented with 10% fetal bovine serum, 2 mM glutamine, 180 units/ml penicillin, and 45 g/ml streptomycin. Transient transfection of the COS-7 cells for cAMP accumulation was performed using the calcium phosphate precipitation method with the addition of chloroquine⁴⁶⁻⁴⁷.

HEK293 cells were cultured in 10% CO₂ and 37° C. in Dulbecco's modified Eagle's medium supplemented with 10% fetal bovine serum, 2 mM glutamine, 180 units/ml penicillin, and 45 g/ml streptomycin. Transient transfection with hGIPR for the CisBio assay from Table 2C was performed by using Lipofectamine 2000.

cAMP Assay

### Alternative 1 (Also Referred to as CisBio Assay):

The in vitro functional activity of compounds towards human GIP receptor can also be determined in HEK-293 cells transiently expressing the receptor. On the day of the assay, cells were resuspended in HBSS buffer (Gibco, 14025-50) supplemented with 20 mM HEPES (Gibco, 15630-106), 0.1% Pluronic F-68 (Gibco, 24040-032) and 0.1% casein (Sigma, C4765), and plated in 384-well plates at a density of 5000 cells/well. The GIP peptide analogues of the present disclosure were diluted in HBSS buffer supplemented with 20 mM HEPES, 0.1% pluronic, 0.1% casein and 500 uM IBMX. To test for antagonistic properties, the GIP peptide analogues to be tested were each independently added to the cells and incubated for 20 min. at 37° C. prior to addition of agonist (GIP1-42) at an EC50 concentration, and subsequent incubation at 37° C. for 30 min. The resulting decrease in intracellular cAMP was quantitatively determined using the CisBio cAMP Dynamic 2 HTRF Assay Kit. The assay is based on a competition between native cAMP produced by cells and cAMP labeled with the dye d2 for binding to a cryptate labeled antibody. The specific signal (i.e. energy transfer signal) is inversely proportional to the concentration of cAMP in the sample.

The cAMP-d2 conjugate and the antibody anti-cAMP-Cryptate, both diluted in lysis buffer provided in the kit, were added to the cells according to the manufacturer's protocol. The resulting competitive assay was incubated for 60 minutes at room temperature, and the signal was detected by using a PerkinElmer Envision® instrument with excitation at 320 nm and emission at 665 nm and 620 nm. The HTRF ratio (emission at 665 nm/620 nm*10,000) is inversely proportional to the amount of cAMP present and is converted to nM cAMP per well using a cAMP standard curve. The dose-response curves were fitted using the non-linear regression analysis (four-logistic parameter equation) in GraphPad Prism, whereby pIC50 values were estimated.

To test for agonistic properties at the GIP receptor, compounds were diluted and added to cells as described above and incubated for 30 min at 37° C. The resulting increase in intracellular cAMP was determined using the CisBio cAMP Dynamic 2 HTRF Assay Kit as described above.

The dose-response curves were fitted using the non-linear regression analysis (four-logistic parameter equation) in GraphPad Prism, whereby pIC50 values were estimated.

### Alternative 2 (Also Referred to as Gaddum Assay):

Antagonist potency (pKb) was estimated in a functional setting. The estimated pKb values were calculated from the shifts of the agonist-concentration-response curves in the presence of a single dose of GIP peptide antagonists using the Gaddum equation: pKb=log(DR−1)−log(B), where DR (EC50′/EC50) is the dose ratio calculated from EC50 of GIP1-42 obtained in the presence and absence (EC50) of antagonist, and B is the antagonist concentration used.

The in vitro functional assessment of compounds towards human GIP receptor was determined in HEK-293 cells transiently expressing the receptor. On the day of the assay, cells were resuspended in HBSS buffer (Gibco, 14025-50) supplemented with 20 mM HEPES (Gibco, 15630-106), 0.1% Pluronic F-68 (Gibco, 24040-032) and 0.1% casein (Sigma, C4765), and plated in 384-well plates at a density of 3500 cells/well. The GIP peptide analogues of the present disclosure were diluted in HBSS buffer supplemented with 20 mM HEPES, 0.1% pluronic, 0.1% casein and 500 uM IBMX. GIP peptide analogues to be tested were each independently added to the cells at a concentration of 3.16 nM for compound AT705-AT718, 31.6 nM for compound AT719-AT725 and AT745-AT755, and 100 nM for compound AT739-AT744, and incubated for 20 min. at 37° C. Increasing dose of agonist (GIP1-42) was subsequently added to cells and incubated for additionally 30 min at 37° C. The intracellular cAMP was quantitatively determined using the CisBio cAMP Dynamic 2 HTRF Assay Kit. The assay is based on a competition between native cAMP produced by cells and cAMP labeled with the dye d2 for binding to a cryptate labeled antibody. The specific signal (i.e. energy transfer signal) is inversely proportional to the concentration of cAMP in the sample.

The cAMP-d2 conjugate and the antibody anti-cAMP-Cryptate, both diluted in lysis buffer provided in the kit, were added to the cells according to the manufacturer's protocol. The resulting competitive assay was incubated for 60 minutes at room temperature, and the signal was detected by using a PerkinElmer Envision® instrument with excitation at 320 nm and emission at 665 nm and 620 nm. The HTRF ratio (emission at 665 nm/620 nm*10,000) is inversely proportional to the amount of cAMP present and is converted to nM cAMP per well using a cAMP standard curve.

Alternative 3 (also referred as Schild analysis) Antagonist potency was also determined by Schild analysis. GIP1-42 EC50 was measured in the absence of antagonist and EC50′ was measured in the presence of increasing concentrations of antagonist. These values were used to calculate a dose ratio (DR=EC50′/EC50) for each antagonist concentration, and log(DR−1) was plotted against log(antagonist concentration). The slope of the resulting line was fixed to 1, and pKb was determined as the intercept with the abscissa.

The in vitro functional assessment of compounds towards human GIP receptor was determined in HEK-293 cells transiently expressing the receptor. On the day of the assay, cells were resuspended in HBSS buffer (Gibco, 14025-50) supplemented with 20 mM HEPES (Gibco, 15630-106), 0.1% Pluronic F-68 (Gibco, 24040-032) and 0.1% casein (Sigma, C4765), and plated in 384-well plates at a density of 3500 cells/well. The GIP peptide analogues of the present disclosure were diluted in HBSS buffer supplemented with 20 mM HEPES, 0.1% pluronic, 0.1% casein and 500 uM IBMX. GIP peptide analogues to be tested were each independently added to the cells at concentrations of 10, 100, and 1000 nM for GIP3-30, AT759, at concentrations of 3.16, 31.6 and 316 nM for AT158, AT364, AT760, AT761, at concentrations of 1, 10, and 100 nM for compound AT762 and AT763, and at concentrations of 31.6, 316 and 3160 nM for AT758, following incubation for 20 min. at 37° C. Increasing dose of agonist (GIP1-42) was subsequently added to cells and incubated for additionally 30 min at 37° C. The intracellular cAMP was quantitatively determined using the CisBio cAMP Dynamic 2 HTRF Assay Kit. The assay is based on a competition between native cAMP produced by cells and cAMP labeled with the dye d2 for binding to a cryptate labeled antibody. The specific signal (i.e. energy transfer signal) is inversely proportional to the concentration of cAMP in the sample.

The cAMP-d2 conjugate and the antibody anti-cAMP-Cryptate, both diluted in lysis buffer provided in the kit, were added to the cells according to the manufacturer's protocol. The resulting competitive assay was incubated for 60 minutes at room temperature, and the signal was detected by using a PerkinElmer Envision® instrument with excitation at 320 nm and emission at 665 nm and 620 nm. The HTRF ratio (emission at 665 nm/620 nm*10,000) is inversely proportional to the amount of cAMP present and is converted to nM cAMP per well using a cAMP standard curve. The dose-response curves were fitted using the non-linear regression analysis (four-logistic parameter equation) in GraphPad Prism, whereby pIC50 values were estimated.

The GIP peptide analogues were also assessed functionally, where potency was determined using the same assay as described above, but with few modifications. Functional assessment was measured in CHO cells stably transfected with the GIPR. Cells were resuspended in HBSS buffer containing 5 mM HEPES, 0.1% casein and 500 uM IBMX. Increasing dose of GIP analogues to be tested were each independently added to cells, and incubated at 37° C. for 20 min prior to addition of agonist (GIP1-42) at an EC50-EC80 concentration, and subsequent incubation at 37° C. for 30 min. The resulting decrease in cAMP was quantified as described in the section above.

TABLE 1
Name and structure of the GIP peptide analogues including some reference peptides for comparison.
When the linker consists of more than one unit, it is intended that the first named unit
is linked to peptide, and the last named unit is linked to the fatty acid:

| | | C-term | FA | | | |
|---|---|---|---|---|---|---|
| ID | Backbone | mod | posi. | FA linker | FA | Mutations |
| AT158 | GIP(3-30) | NH2 | 18 | none | C16-diacid | [H18K] |
| AT482 | GIP(3-30) | NH2 | 18 | none | C16-diacid | [D9E; D15E; H18K; D21E; N24Q] |
| AT437 | GIP(3-30) + | COOH | 18 | 2xAEEAc + y-glu | C18-diacid | [E3S; H18K] |
| | Cex | | | | | |
| AT364 | GIP(3-30) + | COOH | 18 | none | C16diacid | [H18K] |
| | Cex(31-39) | | | | | |
| AT614 | GIP(3-30) | COOH | 18 | none | C16-diacid | [D9E; D15E; H18K; D21E; N24E] |
| | Cex(31-39) | | | | | |
| AT616 | GIP(3-30) | COOH | 18 | none | C16-diacid | [D9E; A13Aib; D15E; H18K; N24E] |
| | Cex(31-39) | | | | | |
| AT617 | GIP(3-30) | COOH | 18 | none | C16-diacid | [H18K; N24E] |
| | Cex(31-39) | | | | | |
| AT623 | GIP(3-30) | COOH | 18 | none | C16-diacid | [H18K; N24E; K30A] |
| | Cex(31-39) | | | | | |
| AT665 | GIP(3-30) | COOH | 18 | none | C16-diacid | [A13Aib; H18K; N24E] |
| | Cex(31-39) | | | | | |
| AT666 | GIP(3-30) | COOH | 18 | none | C16-diacid | [A13Aib; M14L; H18K; N24E] |
| | Cex(31-39) | | | | | |
| AT667 | GIP(3-30) | COOH | 18 | none | C16-diacid | [A13Aib; M14Nle; H18K; N24E] |
| | Cex(31-39) | | | | | |
| AT668 | GIP(3-30) | COOH | 18 | none | C16-diacid | [M14L; H18K; N24E] |
| | Cex(31-39) | | | | | |
| AT669 | GIP(3-30) | COOH | 18 | none | C16-diacid | [M14Nle; H18K; N24E] |
| | Cex(31-39) | | | | | |
| AT670 | GIP(3-30) | COOH | 18 | none | C16-diacid | [M14K; H18K; N24E] |
| | Cex(31-39) | | | | | |
| AT673 | GIP(3-30) | COOH | 18 | none | C16-diacid | [D9E; A13Aib; M14L; D15E; |
| | Cex(31-39) | | | | | H18K; D21E; N24E] |
| AT674 | GIP(3-30) | COOH | 18 | none | C16-diacid | [D9E; A13Aib; M14Nle; |
| | Cex(31-39) | | | | | D15E; H18K; D21E; N24E] |
| AT677 | GIP(3-30) | COOH | 18 | none | C16-diacid | [E3Glutaric acid; H18K] |
| | Cex(31-39) | | | | | |
| AT682 | GIP(3-30) | COOH | 18 | 2xAEEAc + yGlu | C18-diacid | [M14L; H18K; N24E] |
| | Cex(31-39) | | | | | |
| AT683 | GIP(3-30) | COOH | 18 | 2xAEEAc + yGlu | C18-diacid | [M14Nle; H18K; N24E] |
| | Cex(31-39) | | | | | |
| AT685 | GIP(3-30) | COOH | 18 | 2xAEEAc + yGlu | C18-diacid | [A13Aib; M14L; H18K; N24E] |
| | Cex(31-39) | | | | | |
| AT686 | GIP(3-30) | COOH | 18 | 2xAEEAc + yGlu | C18-diacid | [A13Aib; M14Nle; H18K; N24E] |
| | Cex(31-39) | | | | | |
| AT693 | GIP(3-30) | COOH | 18 | 2xAEEAc + yGlu | C18-diacid | [D9E; M14L; D15E; H18K; |
| | Cex(31-39) | | | | | D21E; N24E] |
| AT694 | GIP(3-30) | COOH | 18 | 2xAEEAc + yGlu | C18-diacid | [D9E; M14Nle; D15E; H18K; |
| | Cex(31-39) | | | | | D21E; N24E] |
| AT695 | GIP(3-30) | COOH | 18 | 2xAEEAc + yGlu | C18-diacid | [D9E; A13Aib; M14L; D15E; |
| | Cex(31-39) | | | | | H18K; D21E; N24E] |
| AT696 | GIP(3-30) | COOH | 18 | 2xAEEAc + yGlu | C18-diacid | [D9E; A13Aib; M14Nle; D15E; |
| | Cex(31-39) | | | | | H18K; D21E; N24E] |
| AT700 | GIP(3-30) | COOH | 18 | none | C16-diacid | [E3Glutaric acid; M14Nle; |
| | Cex(31-39) | | | | | H18K; N24E] |
| AT702 | GIP(3-30) | COOH | 18 | 2xAEEAc + yGlu | C18-diacid | [E3Glutaric acid; M14Nle; |
| | Cex(31-39) | | | | | H18K; N24E] |
| AT705 | GIP(3-30) | COOH | 18 | 2xAEEAc + yGlu | C18-diacid | [E3Glutaric acid; D9E; A13Aib; |
| | Cex(31-39) | | | | | M14L; D15E; H18K; D21E; N24E] |
| AT706 | GIP(3-30) | COOH | 18 | 2xAEEAc + yGlu | C18-diacid | [E3Glutaric acid; D9E; A13Aib; |
| | Cex(31-39) | | | | | M14Nle; D15E; H18K; D21E; N24E] |
| AT707 | GIP(3-30) | COOH | 18 | 2xAEEAc + yGlu | C18-diacid | [E3Glutaric acid; D9E; M14L; |
| | Cex(31-39) | | | | | D15E; H18K; D21E; N24E] |
| AT708 | GIP(3-30) | COOH | 18 | 2xAEEAc + yGlu | C18-diacid | [E3Glutaric acid; D9E; A13Aib; |
| | Cex(31-39) | | | | | D15E; H18K; D21E; N24E] |
| AT709 | GIP(3-30) | COOH | 18 | 2xAEEAc + yGlu | C18-diacid | [D9E; A13Aib; D15E; H18K; |
| | Cex(31-39) | | | | | D21E; N24E] |
| AT710 | GIP(3-30) | COOH | 18 | 2xAEEAc + yGlu | C18-diacid | [D9E; D15E; H18K; D21E; N24E] |
| | Cex(31-39) | | | | | |
| AT712 | GIP(3-30) | COOH | 18 | 2xAEEAc + yGlu | C18-diacid | [D9E; A13Aib; M14L; H18K; N24E] |
| | Cex(31-39) | | | | | |
| AT713 | GIP(3-30) | NH2 | 18 | 2xAEEAc + yGlu | C18-diacid | [E3Glutaric acid; D9E; A13Aib; |
| | Cex(31-39) | | | | | M14L; D15E; D21E; H18K; N24E] |
| AT714 | GIP(3-30) | NH2 | 18 | 2xAEEAc + yGlu | C18-diacid | [D9E; A13Aib; M14L; D15E;; |
| | Cex(31-39) | | | | | H18K; D21E N24E] |
| AT715 | GIP(3-30) | COOH | 18 | none | C16-diacid | [E3Glutaric acid; A13Aib; |
| | Cex(31-39) | | | | | H18K; N24E] |
| AT716 | GIP(3-30) | COOH | 18 | none | C16-diacid | [D9E, A13Aib; D15E; H18K; N24E] |
| | Cex(31-39) | | | | | |
| AT717 | GIP(3-30) | COOH | 18 | none | C16-diacid | [E3Glutaric acid; D9E; A13Aib; |
| | Cex(31-39) | | | | | M14L; D15E; H18K; D21E; N24E] |
| AT718 | GIP(3-30) | COOH | 18 | none | C16-diacid | [D9E; M14L; D15E; H18K; D21E; |
| | Cex(31-39) | | | | | N24E] |
| AT719 | GIP(3-30) | COOH | 18 | none | C16-diacid | [H18K] |
| AT720 | GIP(3-30) | COOH | 18 | Aib | C16-diacid | [D9E; A13Aib; M14L; D15E; |
| | Cex(31-39) | | | | | H18K; D21E; N24E] |
| AT721 | GIP(3-30) | COOH | 18 | yGlu | C16-diacid | [D9E; A13Aib; M14L; D15E; |
| | Cex(31-39) | | | | | H18K; D21E; N24E] |
| AT722 | GIP(3-30) | COOH | 18 | SGGGG | C16-diacid | [D9E; A13Aib; M14L; D15E; |
| | Cex(31-39) | | | | | H18K; D21E; N24E] |
| AT724 | GIP(3-30) | COOH | 18 | KAAAEKAAAEKAAAE | C16-diacid | [D9E; A13Aib; M14L; D15E; |
| | Cex(31-39) | | | | | H18K; D21E; N24E] |
| AT725 | GIP(3-30) | COOH | 18 | AELA | C16-diacid | [D9E; A13Aib; M14L; D15E; |
| | Cex(31-39) | | | | | H18K; D21E; N24E] |
| AT739 | GIP(3-30) | COOH | 12 | none | C16-diacid | [I12K; N24E] |
| | Cex(31-39) | | | | | |
| AT740 | GIP(3-30) | COOH | 13 | none | C16-diacid | [A13K; N24E] |
| | Cex(31-39) | | | | | |
| AT741 | GIP(3-30) | COOH | 16 | none | C16-diacid | [N24E] |
| | Cex(31-39) | | | | | |
| AT742 | GIP(3-30) | COOH | 17 | none | C16-diacid | [I17K; N24E] |
| | Cex(31-39) | | | | | |
| AT743 | GIP(3-30) | COOH | 34 | none | C16-diacid | [G34K; N24E] |
| | Cex(31-39) | | | | | |
| AT744 | GIP(3-30) | COOH | 40 | none | C16-diacid | [40K; N24E] |
| | Cex(31-39) | | | | | |
| AT745 | GIP(3-30) | COOH | 18 | none | C16-diacid | [H18K; D21A; N24E] |
| | Cex(31-39) | | | | | |
| AT746 | GIP(3-30) | COOH | 18 | none | C16-diacid | [S11A; H18K; N24E] |
| | Cex(31-39) | | | | | |
| AT747 | GIP(3-30) | COOH | 18 | none | C16-diacid | [H18K; N24E; Q29E] |
| | Cex(31-39) | | | | | |
| AT748 | GIP(3-30) | COOH | 18 | none | C16-diacid | [K16A; H18K; N24E] |
| | Cex(31-39) | | | | | |
| AT749 | GIP(3-30) | COOH | 18 | none | C16-diacid | [H18K; N24E] |
| AT750 | GIP(3-30) | COOH | 18 | none | C20-diacid | [H18K; N24E] |
| | Cex(31-39) | | | | | |
| AT751 | GIP(3-30) | COOH | 18 | none | C16 | [H18K; N24E] |
| | Cex(31-39) | | | | | |
| AT752 | GIP(3-30) | COOH | 18 | 2xAEEAc + yGlu | C18-diacid | [E3Succinic acid; D9E; A13Aib; |
| | Cex(31-39) | | | | | M14L; D15E; H18K; D21E; N24E] |
| AT753 | GIP(3-30) | COOH | 18 | 2xAEEAc + yGlu | C18-diacid | [E3Adipic acid; D9E; A13Aib; |
| | Cex(31-39) | | | | | M14L; D15E; H18K; D21E; N24E] |
| AT754 | GIP(3-30) | COOH | 18 | 2xAEEAc + yGlu | C18-diacid | [D9E; A13Aib; M14L; D15E; |
| | Cex(31-39) | | | | | H18K; N24E] |
| AT755 | GIP(3-30) | COOH | 18 | none | C16-diacid | [E3Glutaric acid; D9E; A13Aib; |
| | Cex(31-39) | | | | | M14Nle; D15E; H18K; D21E; N24E] |
| AT760 | GIP(3-30) | COOH | 18 | none | C16-diacid | [A13Aib; H18K] |
| | Cex(31-39) | | | | | |
| AT762 | GIP(3-30) | COOH | 18 | none | C16-diacid | [E3Glutaric acid; H18K; N24E] |
| | Cex(31-39) | | | | | |
| AT763 | GIP(3-30) | COOH | 18 | none | C16-diacid | [E3Glutaric acid; A13Aib; H18K] |
| | Cex(31-39) | | | | | |

TABLE 2A
Antagonistic effect of reference GIP peptides. The CisBio
Assay (Alternative 1 above) was used to determine antagonistic
of the GIP peptide analogues listed in Table 2A:

| | cAMP antagonism | | |
|---|---|---|---|
| ID | pIC50 | SD | nM |
| GIP(3-30) | 7.5 | 0.2 | 33.8 |
| AT158 | 7.8 | 0.3 | 14.3 |
| AT364 | 9.0 | 0.3 | 1.12 |
| AT437 | 7.5 | 0.2 | 35.5 |
| AT482 | 7.9 | 0.4 | 14.1 |

TABLE 2B
Antagonistic effect of GIP peptide analogues including
some reference peptides. The CisBio Assay (Alternative
1 above) was used to determine antagonistic of the
GIP peptide analogues listed in Table 2B:

cAMP antagonism

| ID | pIC50 | SD | nM | % inhib | n |
|---|---|---|---|---|---|
| AT614 | 9.0 | 0.2 | 1.08 | 100 | 3 |
| AT616 | 9.3 | 0.5 | 0.473 | 100 | 4 |
| AT617 | 9.1 | 0.4 | 0.794 | 91 | 3 |
| AT623 | 8.5 | 0.3 | 3.16 | 98 | 2 |
| AT665 | 9.8 | 0.9 | 0.150 | 88 | 3 |
| AT666 | 9.6 | 0.5 | 0.266 | 89 | 13 |
| AT667 | 9.1 | 0.8 | 0.736 | 86 | 3 |
| AT668 | 9.0 | 0.7 | 0.944 | 92 | 4 |
| AT673 | 9.9 | 0.5 | 0.136 | 94 | 3 |
| AT674 | 9.4 | 0.1 | 0.447 | 93 | 3 |
| AT677 | 9.4 | 0.4 | 0.447 | 93 | 3 |
| AT682 | 7.9 | 0.2 | 13.6 | 89 | 3 |
| AT683 | 7.8 | 0.1 | 15.9 | 93 | 3 |
| AT685 | 8.7 | 0.3 | 1.995 | 83 | 4 |
| AT686 | 8.5 | 0.3 | 3.415 | 82 | 5 |
| AT693 | 8.9 | 0.3 | 1.26 | 96 | 3 |
| AT694 | 8.8 | 0.4 | 1.47 | 88 | 3 |
| AT695 | 9.1 | 0.3 | 0.794 | 98 | 3 |
| AT696 | 9.2 | 0.4 | 0.584 | 97 | 3 |
| AT700 | 9.2 | 0.4 | 0.708 | 89 | 3 |
| AT702 | 8.5 | 0.1 | 3.55 | 85 | 3 |

TABLE 2C
Antagonistic effect of GIP peptide analogues and some
reference peptides for comparison. The Gaddum Assay (Alternative
2 above) was used to determine antagonistic effect of
the GIP peptide analogues listed in Table 2C:

| ID | pKb | SD | nM | N |
|---|---|---|---|---|
| AT705 | 9.6 | 0.4 | 0.23 | 4 |
| AT706 | 9.6 | 0.3 | 0.26 | 4 |
| AT707 | 9.6 | 0.2 | 0.28 | 4 |
| AT708 | 9.7 | 0.3 | 0.20 | 3 |
| AT709 | 9.6 | 0.3 | 0.24 | 4 |
| AT710 | 9.1 | 0.4 | 0.73 | 4 |
| AT712 | 9.4 | 0.2 | 0.39 | 3 |
| AT713 | 9.5 | 0.1 | 0.31 | 2 |
| AT714 | 9.4 | 0.3 | 0.43 | 3 |
| AT715 | 9.7 | 0.6 | 0.19 | 3 |
| AT716 | 9.5 | 0.7 | 0.29 | 3 |
| AT717 | 9.8 | 0.6 | 0.17 | 3 |
| AT718 | 9.5 | 0.1 | 0.33 | 3 |
| AT719 | 8.1 | 0.4 | 7.6 | 3 |
| AT720 | 9.5 | 0.2 | 0.29 | 6 |
| AT721 | 9.5 | 0.2 | 0.31 | 2 |
| AT722 | 9.6 | 0.1 | 0.28 | 2 |
| AT724 | 9.5 | 0.1 | 0.31 | 3 |
| AT725 | 9.2 | 0.2 | 0.60 | 2 |
| AT739 | 7.9 | 0.2 | 11 | 4 |
| AT740 | 7.9 | 0.3 | 14 | 4 |
| AT741 | 7.9 | 0.6 | 11 | 3 |
| AT742 | 7.5 | 0.1 | 32 | 3 |
| AT743 | 7.7 | 0.4 | 19 | 3 |
| AT744 | 8.3 | 0.3 | 4.8 | 4 |
| AT745 | 9.3 | 0.1 | 0.49 | 3 |
| AT746 | 9.0 | 0.3 | 0.97 | 2 |
| AT747 | 7.9 | 0.1 | 14 | 2 |
| AT748 | 9.4 | 0.3 | 0.42 | 3 |
| AT749 | 8.7 | 0.1 | 2.2 | 2 |
| AT750 | 8.4 | 0.0 | 4.1 | 2 |
| AT751 | 9.0 | 0.2 | 0.92 | 3 |
| AT752 | 9.3 | 0.1 | 0.52 | 4 |
| AT753 | 9.6 | 0.2 | 0.23 | 3 |
| AT754 | 8.7 | 0.4 | 2.2 | 2 |
| AT755 | 9.6 | 0.2 | 0.23 | 3 |

TABLE 2D

| ID | pKb | SEM | nM | N |
|---|---|---|---|---|
| GIP3-30 | 8.5 | 0.1 | 3.5 | 5 |
| AT158 | 8.4 | 0.1 | 3.7 | 3 |
| AT364 | 9.1 | 0.1 | 0.77 | 3 |
| AT758 | 8.6 | 0.1 | 2.8 | 4 |
| AT759 | 8.8 | 0.1 | 1.5 | 5 |
| AT760 | 9.3 | 0.1 | 0.46 | 3 |
| AT761 | 8.9 | 0.1 | 1.4 | 3 |
| AT762 | 9.8 | 0.1 | 0.15 | 6 |
| AT763 | 9.9 | 0.1 | 0.14 | 3 |

### Results

It can be seen from Table 2B and 2C that A13Aib substitutions in GIP peptide analogues may increase the antagonistic effect at the GIP receptor.

### Solubility and Physical Stability

### Assessment of Physical Stability

Aggregation in the form of fibril formation was detected using the amyloid-specific dye Thioflavin T (ThT), which is frequently employed to demonstrate the presence of fibrils in solution.

ThT fluoresces weakly at around 527 nm but displays a red-shift in the emission spectrum to around 486 nm and an increase in intensity of emitted light upon binding to beta sheet rich structures. Continuous measurement of the fluorescence emission at 486 nm can be used as a measure of fibrillation behavior of peptides and proteins. The time until onset of fibrillation or fibrillation lag time is estimated here by defining the lag time (Tlag) as the point in time where the signal relative to the pre-transition baseline has reached 10% of the post-transition baseline.

### Chemicals:

Sodium phosphate dibasic (Na₂HPO₄ anhydrous, Sigma, Lot: SLBL9126V) Sodium phosphate monobasic (NaH₂PO₄, anhydrous, Sigma, Lot: SLBP1516V) 50 mM sodium phosphate buffer for dissolving some samples was prepared in ultrapure water (Milli-Q® Reference A+ System, Merck) with a resistivity of 18.2 MΩ·cm. The buffer was adjusted to pH 7.4 and filtered.

The ultrapure water (MilliQ) for dissolving some samples was adjusted to pH 7.4 with NaOH and filtered prior to sample preparation.

### Sample Preparation

Peptides were either dissolved in sodium phosphate buffer (50 mM, pH 7.4, filtered) or dissolved in ultrapure (MilliQ) water (adjusted to pH 7.4 with NaOH, filtered) (see Table 3). All peptide samples were prepared to a concentration of 1, 5, 7.5 or 15 mg/ml. All samples, with the exception of reference peptides GIP(3-30), AT158, and AT482 dissolved readily and produced clear and colorless solutions with gentle mixing. Peptide samples were subsequently filtered through 0.22 μm nylon filters (Q-Max® RR syringe filter, 13 mm, Frisenette, DK) to produce particle-free solutions. All samples were added to a 96-well plate reader for the ThT assay at t=0.

For each sample, 22 μL ThT (1 mM) was added to 1.2 mL peptide solution. From this sample mixture, an amount of 200 uL/well was pipetted into four or five wells (n=4 or 5). Blank samples (buffer+ThT) were also included. One 3 mm silica bead was added to each well containing samples and the plate was subjected to orbital rotation at 300 rpm to agitate and stress the samples. The temperature was kept at 25° C. throughout the measurement.

### Platereader Settings:

Excitation wavelength: 450 nm
Dicroic filter: 465 nm
Emission wavelength: 486 nm
Focal height: 3.5 mm
Gain: 1000
Number of cycles: 960
Cycle time: 360 s
Number of flashes per well: 20

### Results

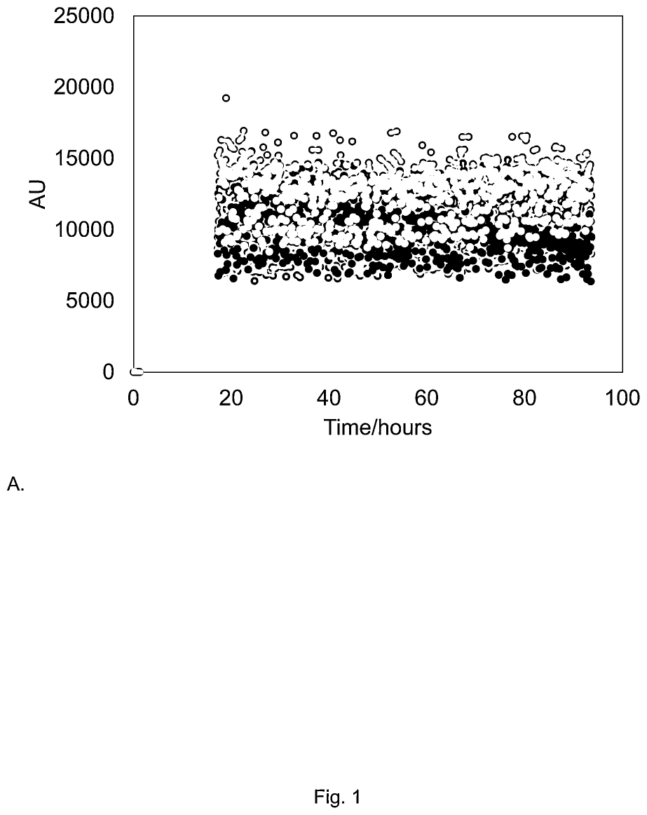

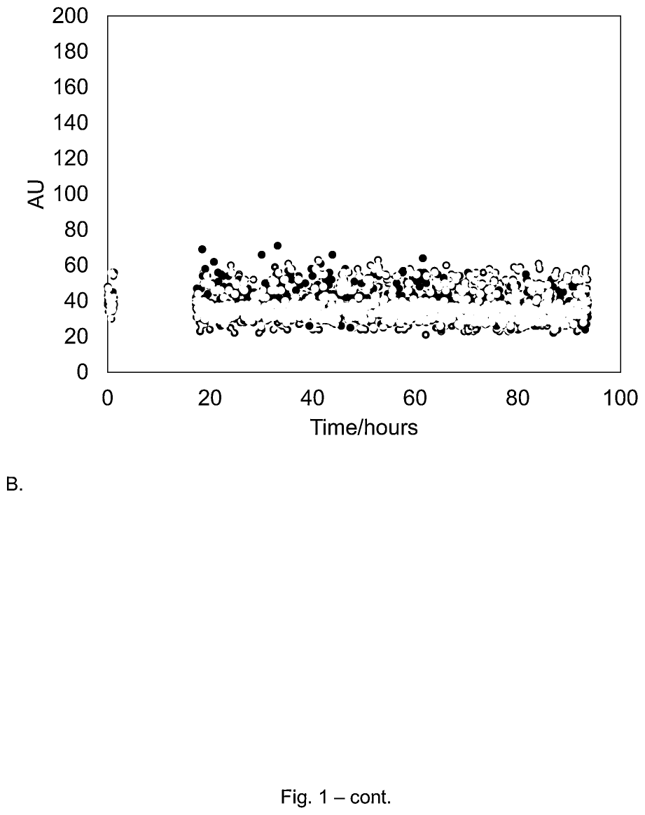

An overview of results can be seen in Table 3. In addition, FIG. 1 shows a comparison between a GIP peptide analogue with high stability (AT763 in phosphate buffer—FIG. 1B) that does not form fibrils, and a reference GIP analogue with lower physical stability (AT364 in phosphate buffer—FIG. 1A), that forms fibrils.

From Table 3, it can be seen that A13Aib and/or N24E substitutions in GIP peptide analogues increase the physical stability of GIP peptide analogues as measured by decreased tendency to fibril formation in a ThT assay. See e.g AT760 vs AT364, and AT762 vs AT677, and AT763 vs AT677. GIP peptide analogues according to embodiments of the invention showed no increase in absorbance intensity over the course of the 96-hour measurement time indicating that the samples did not fibrillate and that the peptides are physical stable in aqueous solution. See e.g AT673, AT695 and AT696 vs AT364, and e.g. AT749 vs AT158 and AT719. See also AT677 versus AT717 and AT755.

It can also be seen from Table 3, that a fatty acid can be attached at different positions, such as 12, 13, 16, 17, 18, 34, and 40, and retain increased physical stability. See e.g. AT739, AT740, AT741, AT742, AT743, AT744 and AT668, which did not fibrillate within 96 hours.

### Assessment of Solubility

Clear visual appearance may be taken as an indicator of the immediate solubility of the peptide. Hence it can be seen that all peptides according to embodiments of the present invention displayed increased solubility as compared to GIP(3-30), AT158, and AT482. Thus, A13Aib and/or N24E substitutions appear to increase solubility.

TABLE 3
Solubility (as measured by visual inspection) and physical stability (as measured
by estimated lag time in a ThT assay) of GIP peptide analogues tested.

| | | | Counter | Buffer | Visual | |
|---|---|---|---|---|---|---|
| Number/ID | Concentration | | ion | (pH) | appearance | Avr lag time (h) |
| GIP(3-30) | 1 | mg/ml | | MilliQ (7.8) | Clear with a | High absorbance |
| | | | | | little sediment | from t = 0, |
| | | | | | | indicating that |
| | | | | | | fibrillation had |
| | | | | | | already started |
| GIP(3-30) | 1 | mg/ml | TFA | Phosphate (7.4) | Very turbid^{#} | |
| AT158 | 1 | mg/ml | Cl | MilliQ (7.9) | Turbid | High absorbance |
| | | | | | | from t = 0, |
| | | | | | | indicating that |
| | | | | | | fibrillation had |
| | | | | | | already started |
| AT158 | 1 | mg/ml | Cl | MilliQ (7.9) | Hazy | High absorbance |
| | | | | | | from t = 0, |
| | | | | | | indicating that |
| | | | | | | fibrillation had |
| | | | | | | already started |
| AT158 | 1 | mg/ml | Acetate | Phosphate (7.4) | Almost clear | High absorbance |
| | | | | | | from t = 0, |
| | | | | | | indicating that |
| | | | | | | fibrillation had |
| | | | | | | already started |
| AT158 | 1 | mg/ml | TFA | Phosphate (7.4) | | <18 h |
| AT158 | 5 | mg/ml | TFA | MilliQ | | <18 h |
| AT364 | 1 | mg/ml | Cl | Phosphate (7.4) | Clear | Steady increase |
| | | | | | | in absorbance |
| | | | | | | from the |
| | | | | | | beginning. No lag |
| | | | | | | time could be |
| | | | | | | estimated. |
| AT364 | 1 | mg/ml | Na | MilliQ | Clear | Slow absorbance |
| | | | | | | increase |
| AT364 | 5 | mg/ml | Na | MilliQ | Clear | 20-50 |
| AT364 | 7.5 | mg/ml | Na | MilliQ (8) | Clear | 17.3 h ± 2.7 |
| AT364 | 15 | mg/ml | Na | MilliQ (8) | Clear | 7.4 h ± 0.4 |
| AT364 | 1 | mg/ml | TFA | Phosphate 7.4 | Clear | <18 h |
| AT364 | 5 | mg/ml | TFA | Phosphate | Clear | <18 h |
| AT364 | 5 | mg/ml | TFA | MilliQ (8) | Clear | <18 h |
| AT437 | 1 | mg/ml | TFA | Phosphate (7.4) | Clear | ~56 |
| AT482 | 1 | mg/ml | TFA | Phosphate (7.4) | White/turbid | High absorbance |
| | | | | | | from t = 0, |
| | | | | | | indicating that |
| | | | | | | fibrillation had |
| | | | | | | already started |
| AT616 | 1 | mg/ml | Na | MilliQ (7.7) | Clear | fnd |
| AT665 | 1 | mg/ml | TFA | Phosphate (7.4) | Clear | fnd |
| AT665 | 5 | mg/ml | Na | MilliQ (8) | | fnd |
| AT665 | 7.5 | mg/ml | Na | MilliQ (8) | Clear | fnd |
| AT665 | 15 | mg/ml | Na | MilliQ (8) | Clear | fnd |
| AT666 | 1 | mg/ml | TFA | Phosphate (7.4) | Clear | fnd |
| AT668 | 1 | mg/ml | TFA | Phosphate (7.4) | Clear | fnd |
| AT673 | 1 | mg/ml | TFA | Phosphate (7.4) | Clear | fnd |
| AT673 | 5 | mg/ml | Na | MilliQ (8) | Clear | fnd |
| AT673 | 7.5 | mg/ml | Na | MilliQ (8) | Clear | fnd |
| AT673 | 15 | mg/ml | Na | MilliQ (8) | Clear | fnd |
| AT674 | 1 | mg/ml | TFA | Phosphate (7.4) | Clear | fnd |
| AT677 | 1 | mg/ml | TFA | Phosphate (7.4) | Clear | 16.5 +/− 2.3 |
| AT677 | 1 | mg/ml | Na | MilliQ (8) | | <24 |
| AT685 | 1 | mg/ml | TFA | Phosphate (7.4) | Clear | fnd |
| AT693 | 1 | mg/ml | TFA | Phosphate (7.4) | Clear | fnd |
| AT694 | 1 | mg/ml | TFA | Phosphate (7.4) | Clear | fnd |
| AT695 | 1 | mg/ml | TFA | Phosphate (7.4) | Clear | fnd |
| AT695 | 5 | mg/ml | Na | MilliQ (8) | Clear | fnd |
| AT696 | 1 | mg/ml | TFA | Phosphate (7.4) | Clear | fnd |
| AT696 | 5 | mg/ml | Na | MilliQ (8) | Clear | fnd |
| AT696 | 7.5 | mg/ml | Na | MilliQ (8) | Clear | fnd |
| AT696 | 15 | mg/ml | Na | MilliQ (8) | Clear | fnd |
| AT705 | 1 | mg/ml | TFA | Phosphate (7.4) | Clear | fnd |
| AT706 | 1 | mg/ml | TFA | Phosphate (7.4) | Clear | fnd |
| AT706 | 7.5 | mg/ml | Na | MilliQ (8) | Clear | fnd |
| AT706 | 15 | mg/ml | Na | MilliQ (8) | Clear | fnd |
| AT712 | 1 | mg/ml | TFA | Phosphate (7.4) | Clear | fnd |
| AT713 | 1 | mg/ml | TFA | Phosphate (7.4) | Clear | fnd |
| AT713 | 7.5 | mg/ml | Na | MilliQ (8) | Clear | fnd |
| AT713 | 15 | mg/ml | Na | MilliQ (8) | Clear | fnd |
| AT714 | 1 | mg/ml | TFA | Phosphate (7.4) | Clear | fnd |
| AT717 | 1 | mg/ml | TFA | Phosphate (7.4) | Clear | fnd |
| AT717 | 7.5 | mg/ml | Na | MilliQ (8) | Clear | fnd |
| AT717 | 15 | mg/ml | Na | MilliQ (8) | Clear | fnd |
| AT719 | 1 | mg/ml | TFA | Phosphate (7.4) | Clear | <8 |
| AT720 | 7.5 | mg/ml | Na | MiliQ (8) | Clear | fnd |
| AT720 | 15 | mg/ml | Na | MiliQ (8) | Clear | fnd |
| AT721 | 7.5 | mg/ml | Na | MilliQ (8) | Clear | fnd |
| AT721 | 15 | mg/ml | Na | MilliQ (8) | Clear | fnd |
| AT722 | 1 | mg/ml | TFA | Phosphate (7.4) | Clear | fnd |
| AT722 | 7.5 | mg/ml | Na | MilliQ (8) | Clear | fnd |
| AT722 | 15 | mg/ml | Na | MilliQ (8) | Clear | fnd |
| AT724 | 7.5 | mg/ml | Na | MiliQ (8) | Clear | fnd |
| AT724 | 15 | mg/ml | Na | MiliQ (8) | Clear | fnd |
| AT725 | 1 | mg/ml | TFA | Phosphate (7.4) | Clear | fnd |
| AT739 | 1 | mg/ml | TFA | Phosphate (7.4) | Clear | fnd |
| AT740 | 1 | mg/ml | TFA | Phosphate (7.4) | Clear | fnd |
| AT741 | 1 | mg/ml | TFA | Phosphate (7.4) | Clear | fnd |
| AT742 | 1 | mg/ml | TFA | Phosphate (7.4) | Clear | fnd |
| AT743 | 1 | mg/ml | TFA | Phosphate (7.4) | Clear | fnd |
| AT744 | 1 | mg/ml | TFA | Phosphate (7.4) | Clear | fnd |
| AT745 | 1 | mg/ml | TFA | Phosphate (7.4) | Clear | <24 |
| AT746 | 1 | mg/ml | TFA | Phosphate (7.4) | Clear | fnd |
| AT747 | 1 | mg/ml | TFA | Phosphate (7.4) | Clear | <24 |
| AT748 | 1 | mg/ml | TFA | Phosphate (7.4) | Clear | <24 |
| AT749 | 1 | mg/ml | TFA | Phosphate (7.4) | Clear | <24 |
| AT750 | 1 | mg/ml | TFA | Phosphate (7.4) | Clear | fnd |
| AT751 | 1 | mg/ml | TFA | Phosphate (7.4) | Clear | <24 |
| AT754 | 1 | mg/ml | TFA | Phosphate (7.4) | Clear | fnd |
| AT755 | 1 | mg/ml | TFA | Phosphate (7.4) | Clear | fnd |
| AT760 | 1 | mg/ml | TFA | Phosphate | Clear | fnd |
| AT760 | 1 | mg/ml | TFA | Phosphate | Clear | fnd |
| AT760 | 5 | mg/ml | TFA | MilliQ | Clear | fnd |
| AT762 | 1 | mg/ml | TFA | Phosphate | Clear | fnd |
| AT762 | 5 | mg/ml | TFA | MilliQ | Clear | fnd |
| AT763 | 1 | mg/ml | TFA | Phosphate | Clear | fnd |
| AT763 | 5 | mg/ml | TFA | Phosphate | Clear | fnd |
| AT763 | 5 | mg/ml | TFA | MilliQ | Clear | fnd |

Fnd = fibrils not detected within experiment time (96 hours) with agitation.
^{#}Most of the peptide precipitated and was caught in the filter during filtration.

1.-85. (canceled)

86. A glucose-dependent insulinotropic peptide (GIP) analogue consisting of amino acid sequence SEQ ID NO:4:
1 2 3 4 5 6 7 8 9 10 11
X₁ - G - T - F - I - S - D - Y - S - I - Aib -
12 13 14 15 16 17 18 19 20 21 22
M - D - K - I H - Q - Q - D - F - V - E -
23 24 25 26 27 28
W - L - L - A - Q - K - Z,
wherein X₁ is any amino acid or omitted;
or a functional variant thereof, wherein said variant has 1 to 5 individual amino acid substitutions at any amino acid of SEQ ID NO:4,
wherein the amino acid at position 22 of said functional variant is E and wherein the amino acid at position 11 of said functional variant is 2-Aminoisobutyric acid (Aib),
wherein Z is a peptide comprising one or more amino acid residues of Exendin-4(31-39) (PSSGAPPPS; SEQ ID NO:67) or is omitted, and
wherein said peptide is modified by attaching one fatty acid molecule directly or via a linker at one amino acid residue at any position of SEQ ID NO:4 or said functional variant thereof, or directly or via a linker at one amino acid residue at any position of Z, if Z is not omitted.

87. The GIP analogue according to claim 86, wherein said GIP analogue:
(a) is an antagonist of GIPR;
(b) inhibits GIPR activity at least 80%, at least 85%, at least 90%, at least 95%, or about 100%;
(c) inhibits GIPR activity at least 80%, at least 85%, at least 90%, at least 95%, or about 100%, wherein inhibition of GIPR activity is determined as a decrease in intracellular cAMP;
(d) has a GIPR antagonistic potency corresponding to an IC50 value of less than 50 nM, less than 10 nM, less than 5 nM, less than 1 nM, or 0.001 nM to 1 nM;
(e) has improved solubility as compared to GIP(3-30) (SEQ ID NO: 68) and/or as compared to AT364 (SEQ ID NO:6) at pH 7 to 8.5;
(f) has an aqueous solubility of at least 1 mg/ml, at least 5 mg/ml, at least 7.5 mg/ml, at least 10 mg/ml, or at least 15 mg/ml;
(g) has an aqueous solubility at pH about pH 7.5 or about pH 8 of at least 1 mg/ml, at least 5 mg/ml, at least 7.5 mg/ml, at least 10 mg/ml, or at least 15 mg/ml;
(h) has improved physical stability measured by a fibrillation lag time in a ThT assay of more than about 24 hours, more than about 50 hours, more than about 96 hours, or more than about 168 hours; and/or
(i) has improved physical stability as compared to GIP(3-30) (SEQ ID NO: 68) and/or as compared to AT364 (SEQ ID NO:6) at pH 7 to 8.5.

88. The GIP analogue according to claim 86, wherein said peptide is modified by attaching one fatty acid molecule at one amino acid residue at position 1 to 27 of SEQ ID NO:4, or said functional variant thereof.

89. The GIP analogue according to claim 86, wherein:
(a) x₁ at position 1 of SEQ ID NO:4, or said variant thereof, is selected from E, glutaric acid, succinic acid and adipic acid;
(b) the amino acid at position 7 of SEQ ID NO:4, or said variant thereof, is selected from D and E;
(c) the amino acid at position 9 of SEQ ID NO:4, or said variant thereof, is selected from S, K and A;
(d) the amino acid at position 10 of SEQ ID NO:4, or said variant thereof, is selected from I and K;
(e) the amino acid at position 12 of SEQ ID NO:4, or said variant thereof, is selected from M, L and Norleucine (Nle);
(f) the amino acid at position 13 of SEQ ID NO:4, or said variant thereof, is selected from D and E;
(g) the amino acid at position 14 of SEQ ID NO:4, or said variant thereof, is selected from K and R;
(h) the amino acid at position 15 of SEQ ID NO:4, or said variant thereof, is selected from I and K;
(i) the amino acid at position 16 of SEQ ID NO:4, or said variant thereof, is selected from H and K;
(j) the amino acid at position 18 of SEQ ID NO:4, or said variant thereof, is selected from Q and K;
(k) the amino acid at position 19 of SEQ ID NO:4, or said variant thereof, is selected from D and E; and/or
(l) the amino acid at position 4 of Z if present is selected from G and K.

90. The GIP analogue according to claim 86, wherein said functional variant has 1 individual amino acid substitution, 2 individual amino acid substitutions, 3 individual amino acid substitutions, or 4 individual amino acid substitutions, at any amino acid of SEQ ID NO:4.

91. The GIP analogue according to claim 86, wherein at least one amino acid residue of the GIP analogue of SEQ ID NO:4 is substituted with E, or wherein at least one amino acid residue at any one of positions 7, 13, and 19 of SEQ ID NO:4, or said functional variant thereof, is substituted with E.

92. The GIP analogue according to claim 86, wherein:
(a) X₁ is E or glutaric acid;
(b) the D at position 7 of SEQ ID NO:4, or said functional variant thereof, is substituted with any amino acid, or substituted with E;
(c) the S at position 9 of SEQ ID NO:4, or said functional variant thereof, is substituted with any amino acid, a conservative amino acid substitution, or substituted with an amino acid residue selected from the group consisting of A and K;
(d) the I at position 10 of SEQ ID NO:4, or said functional variant thereof, is substituted with any amino acid, a conservative amino acid substitution, or substituted with K;
(e) the M at position 12 of SEQ ID NO:4, or said functional variant thereof, is substituted with any amino acid, a conservative amino acid substitution, or substituted with an amino acid residue selected from the group consisting of L and Norleucine (Nle);
(f) the D at position 13 of SEQ ID NO:4, or said functional variant thereof, is substituted with any amino acid, a conservative amino acid substitution, or substituted with E;
(g) the K at position 14 of SEQ ID NO:4, or said functional variant thereof, is substituted with any amino acid, a conservative amino acid substitution, or substituted with R;
(h) the I at position 15 of SEQ ID NO:4, or said functional variant thereof, is substituted with any amino acid, a conservative amino acid substitution, or substituted with K;
(i) the H at position 16 of SEQ ID NO:4, or said functional variant thereof, is substituted with any amino acid, a conservative amino acid substitution, or substituted with K;
(j) the Q at position 18 of SEQ ID NO:4, or said functional variant thereof, is substituted with any amino acid, a conservative amino acid substitution, or substituted with K;
(k) the D at position 19 of SEQ ID NO:4, or said functional variant thereof, is substituted with any amino acid, a conservative amino acid substitution, or substituted with E; and/or
(l) the G at position 4 of Z, if present, is substituted with any amino acid, a conservative amino acid substitution, or substituted with K.

93. The GIP analogue according to claim 86, wherein said GIP analogue comprises at least one substitution to K and/or at least one substitution to E at any one of amino acid residues 1 to 28 of SEQ ID NO:4.

94. The GIP analogue according to claim 86, wherein Z is a peptide selected from the group consisting of:
(a) a peptide consisting of one or more amino consecutive acid residues of Exendin-4(31-39) (PSSGAPPPS; SEQ ID NO:67),
(b) a glycine or a proline,
(c) GP, GPS, GPSS (SEQ ID NO: 55), GPSSG (SEQ ID NO: 56), GPSSGA (SEQ ID NO: 57), GPSSGAP (SEQ ID NO: 58), GPSSGAPP (SEQ ID NO: 59), GPSSGAPPP (SEQ ID NO: 60) and GPSSGAPPPS (SEQ ID NO: 61),
(d) PS, PSS, PSSG (SEQ ID NO: 62), PSSGA (SEQ ID NO: 63), PSSGAP (SEQ ID NO: 64), PSSGAPP (SEQ ID NO: 65), PSSGAPPP (SEQ ID NO: 66) and PSSGAPPPS (SEQ ID NO: 67),
(e) GPSSGA (SEQ ID NO: 57), GPSSGAP (SEQ ID NO: 58), GPSSGAPP (SEQ ID NO: 59), GPSSGAPPP (SEQ ID NO: 60), GPSSGAPPPS (SEQ ID NO: 61), or a variant thereof comprising 1 or 2 individual amino acid substitutions at any one of the amino acid residues, and
(f) PSSG (SEQ ID NO: 62), PSSGA (SEQ ID NO: 63), PSSGAP (SEQ ID NO: 64), PSSGAPP (SEQ ID NO: 65), PSSGAPPP (SEQ ID NO: 66) and PSSGAPPPS (SEQ ID NO: 67), or a variant thereof comprising 1 or 2 individual amino acid substitutions at any one of the amino acid residues.

95. The GIP analogue according to claim 86, wherein the fatty acid molecule is not attached at the amino acid residue at position 1 or the N-terminal amino group of the amino acid residue at position 1 of SEQ ID NO:4, or said functional variant thereof; and/or
wherein the fatty acid molecule is attached to the side chain of an amino acid residue at position 9, position 10, position 11, position 14, position 15, position 16, position 18, of SEQ ID NO:4, or said functional variant thereof, and/or
wherein said fatty acid molecule is attached to an amino acid residue at any one of positions 10, 11, 14, 15 and 16 of SEQ ID NO:4, or said functional variant thereof.

96. The GIP analogue according to claim 86, wherein:
(a) a fatty acid molecule is attached to the side chain amino group of the amino acid residue at position 16 of SEQ ID NO:4, or said functional variant thereof, wherein H at position 16 is substituted with K or Orn in SEQ ID NO:4, or said functional variant thereof; or
(b) a fatty acid molecule is attached to the side chain amino group of the amino acid residue at position 9 of SEQ ID NO:4, or said functional variant thereof, wherein S at position 9 is substituted with K or Orn in SEQ ID NO:4, or said functional variant thereof; or
(c) a fatty acid molecule is attached to the side chain amino group of the amino acid residue at position 10 of SEQ ID NO:4, or said functional variant thereof, wherein I at position 10 is substituted with K or Orn in SEQ ID NO:4, or said functional variant thereof.

97. The GIP analogue of claim 86, wherein said GIP analogue has an amino acid sequence selected from the group consisting of:
SEQ ID NO: 72
EGTFISEYSIAibMEKIKQQDFVEWLLAQK-Z;;
SEQ ID NO 73
EGTFISDYSIAibMDKIKQQDFVEWLLAQK-Z;;
SEQ ID NO: 74
EGTFISDYSIAibLDKIKQQDFVEWLLAQK-Z;;
SEQ ID NO: 75
EGTFISDYSIAibNleDKIKQQDFVEWLLAQK-Z;;
SEQ ID NO: 79
EGTFISEYSIAibLEKIKQQEFVEWLLAQK-Z;;
SEQ ID NO: 80
EGTFISEYSIAibNleEKIKQQEFVEWLLAQK-Z;;
SEQ ID NO: 84
EGTFISEYSIAibLEKIKQQDFVEWLLAQK-Z;;
SEQ ID NO: 85
XGTFISEYSIAibLEKIKQQEFVEWLLAQK-Z;;
SEQ ID NO: 86
XGTFISEYSIAibNleEKIKQQEFVEWLLAQK-Z;;
SEQ ID NO: 88
XGTFISEYSIAibMEKIKQQEFVEWLLAQK-Z;;
SEQ ID NO: 89
EGTFISEYSIAibMEKIKQQEFVEWLLAQK-Z;;
SEQ ID NO: 91
EGTFISEYSIAibLDKIKQQDFVEWLLAQK-Z;;
SEQ ID NO: 92
XGTFISDYSIAibMDKIKQQDFVEWLLAQK-Z;;
SEQ ID NO: 72
EGTFISEYSIAibMEKIKQQDFVEWLLAQK-Z;;
SEQ ID NO: 98
XGTFISEYSIAibLEKIKQQEFVEWLLAQK-Z;;
and
SEQ ID NO: 99
XGTFISEYSIAibLEKIKQQEFVEWLLAQK-Z;;
or a functional variant thereof, wherein said variant has 1 to 4 individual amino acid substitutions.

98. The GIP analogue according to claim 86, wherein said peptide is C-terminally amidated (—NH₂) and/or C-terminally carboxylated (—COOH).

99. The GIP analogue according to claim 86, wherein said fatty acid molecule:
(a) is a straight-chain fatty acid or a branched fatty acid;
(b) is a monoacyl fatty acid molecule;
(c) is a diacyl fatty acid molecule;
(d) comprises an acyl group of the formula CH₃(CH₂)ₙCO—, wherein n is in an integer from 4 to 24;
(e) comprises an acyl group selected from the group consisting of COOH(CH₂)₁₄CO—, COOH(CH₂)₁₆CO—, COOH(CH₂)₁₈CO— and COOH(CH₂)₂₀CO—;
and/or
(f) is attached to the epsilon amino group of the side chain of an amino acid residue of said GIP analogue.

100. The GIP analogue according to claim 86, wherein said fatty acid molecule is attached to an amino acid residue; wherein said linker comprises one or more moieties individually selected from the group consisting of:
(a) α-amino acid, γ-amino acid or ω-amino acid;
(b) Lys;
(c) one or more amino acids selected from the group consisting of succinic acid, Lys, Glu, Asp;
(d) one or more amino acids selected from the group consisting of Gly and Ser;
(e) one or more amino acids selected from the group consisting of Ala, Glu, Lys and Leu;
(f) one or more of γ-aminobutanoyl (γ-aminobutyric acid), γ-Glu (γ-glutamic acid), β-Asp (P-asparagyl), β-Ala (β-alanyl), 2-aminoisobutyric acid (Aib) and Gly; and
(g) [8-amino-3,6-dioxaoctanoic acid]ₙ (AEEAcₙ), wherein n is an integer between 1 and 50, or an integer between 1-4, 1-3 or 1-2.

101. The GIP analogue according to claim 86, wherein the GIP analogue is selected from the group consisting of:
(a) EGTFISEYSIAibMEKIKQQDFVEWLLAQKPSSGAPPPS-
C16-diacid/16K (SEQ ID NO: 33 C16-diacid/16K),
(b) EGTFISDYSIAibMDKIKQQDFVEWLLAQKPSSGAPPPS-
C16-diacid/16K,(SEQ ID NO: 12 C16-diacid/16K),
(c) EGTFISDYSIAibLDKIKQQDFVEWLLAQKPSSGAPPPS-
C16-diacid/16K (SEQ ID NO: 13 C16-diacid/16K),
(d) EGTFISDYSIAibLDKIKQQDFVEWLLAQKPSSGAPPPS-
2xAEEAc + yGlu-C18-diacid/16K (SEQ ID NO: 13
2xAEEAc + yGlu-C18-diacid/16K),
(e) EGTFISDYSIAibNleDKIKQQDFVEWLLAQKPSSGAPPPS-
C16-diacid/16K (SEQ ID NO: 14 C16-diacid/18K),
(f) EGTFISDYSIAibNleDKIKQQDFVEWLLAQKPSSGAPPPS-
2xAEEAc + yGlu-C18-diacid/16K (SEQ ID NO: 14
2xAEEAc + yGlu-C18-diacid/16K)
(g) EGTFISEYSIAibLEKIKQQEFVEWLLAQKPSSGAPPPS-
C16-diacid/16K (SEQ ID NO: 18 C16-diacid/16K),
(h) EGTFISEYSIAibLEKIKQQEFVEWLLAQKPSSGAPPPS-
2xAEEAc + yGlu-C18-diacid/16K (SEQ ID NO: 18
2xAEEAc + yGlu-C18-diacid/16K),
(i) EGTFISEYSIAibNleEKIKQQEFVEWLLAQKPSSGAPPPS-
C16-diacid/16K (SEQ ID NO: 19 C16-diacid/16K),
(j) EGTFISEYSIAibNleEKIKQQEFVEWLLAQKPSSGAPPPS-
2xAEEAc + yGlu-C18-diacid/16K (SEQ ID NO: 19
2xAEEAc + yGlu-C18-diacid/16K)
(k) XGTFISEYSIAibLEKIKQQEFVEWLLAQKPSSGAPPPS-
2xAEEAc + yGlu-C18-diacid/16K (SEQ ID NO: 25
2xAEEAc + yGlu-C18-diacid/16K),
(l) XGTFISEYSIAibNleEKIKQQEFVEWLLAQKPSSGAPPPS-
2xAEEAc + yGlu-C18-diacid/16K (SEQ ID NO: 26
2xAEEAc + yGlu-C18-diacid/16K),
(m) XGTFISEYSIAibMEKIKQQEFVEWLLAQKPSSGAPPPS-
2xAEEAc + yGlu-C18-diacid/16K (SEQ ID NO: 28
2xAEEAc + yGlu-C18-diacid/16K),
(n) EGTFISEYSIAibMEKIKQQEFVEWLLAQKPSSGAPPPS-
2xAEEAc + yGlu-C18-diacid/16K (SEQ ID NO: 29
2xAEEAc + yGlu-C18-diacid/16K),
(o) EGTFISEYSIAibLDKIKQQDFVEWLLAQKPSSGAPPPS-
2xAEEAc + yGlu-C18-diacid/16K (SEQ ID NO: 30
2xAEEAc + yGlu-C18-diacid/16K),
(p) XGTFISEYSIAibLEKIKQQEFVEWLLAQKPSSGAPPPS-
2xAEEAc + yGlu-C18-diacid/16K (SEQ ID NO: 25
2xAEEAc + yGlu-C18-diacid/16K),
(q) EGTFISEYSIAibLEKIKQQEFVEWLLAQKPSSGAPPPS-
2xAEEAc + yGlu-C18-diacid/16K (SEQ ID NO: 18
2xAEEAc + yGlu-C18-diacid/16K),
(r) XGTFISDYSIAibMDKIKQQDFVEWLLAQKPSSGAPPPS-
C16-diacid/16K (SEQ ID NO: 32 C16-diacid/16K),
(s) EGTFISEYSIAibMEKIKQQDFVEWLLAQKPSSGAPPPS-
C16-diacid/16K (SEQ ID NO: 33 C16-diacid/16K),
(t) XGTFISEYSIAibLEKIKQQEFVEWLLAQKPSSGAPPPS-
C16-diacid/16K (SEQ ID NO: 25 C16-diacid/16K),
(u) EGTFISEYSIAibLEKIKQQEFVEWLLAQKPSSGAPPPS-
GGGS-C16-diacid/16K (SEQ ID NO: 18 GGGS-C16-
diacid/16K),
(v) EGTFISEYSIAibLEKIKQQEFVEWLLAQKPSSGAPPPS-
ALEA-C16-diacid/16K (SEQ ID NO: 18 ALEA-C16-
diacid/16K),
(w) EGTFISEYSIAibLEKIKQQDFVEWLLAQKPSSGAPPPS-
2xAEEAc + yGlu-C18-diacid/16K (SEQ ID NO: 50
2xAEEAc + yGlu-C18-diacid/16K),
(x) XGTFISEYSIAibNleEKIKQQEFVEWLLAQKPSSGAPPPS-
C16-diacid/16K (SEQ ID NO: 26 C16-diacid/16K),
(y) EGTFISEYSIAibLEKIKQQEFVEWLLAQKPSSGAPPPS-
Aib-C16-diacid/16K (SEQ ID NO: 18 Aib-C16-
diacid/16K),
(z) EGTFISEYSIAibLEKIKQQEFVEWLLAQKPSSGAPPPS-
KAAAEKAAAEKAAAE-C16-diacid/16K (SEQ ID NO: 18
KAAAEKAAAEKAAAE-C16-diacid/16K),
(aa) XGTFISEYSIAibLEKIKQQEFVEWLLAQKPSSGAPPPS-
2xAEEAc + yGlu-C18-diacid/16K (SEQ ID NO: 48
2xAEEAc + yGlu-C18-diacid/16K),
(ab) XGTFISEYSIAibLEKIKQQEFVEWLLAQKPSSGAPPPS-
2xAEEAc + yGlu-C18-diacid/16K (SEQ ID NO: 49
2xAEEAc + yGlu-C18-diacid/16K),
or a functional variant thereof, wherein said variant has 1 to 4 individual amino acid substitutions.

102. The GIP analogue according to claim 100, wherein said functional variant has 1, 2 or 3 individual amino acid substitutions.

103. The GIP analogue according to claim 86, wherein said peptide is EGTFISEYSIAibLEKIKQQEFVEWLLAQKPSSGAPPPS-C16-diacid/18K (SEQ ID NO:
174 C16-diacid/18K), or a functional variant thereof, wherein said variant has 1 or 2 individual amino acid substitutions.

104. The GIP analogue according to claim 86, wherein said peptide is XGTFISEYSIAibNleEKIKQQEFVEWLLAQKPSSGAPPPS-2×AEEAc+yGlu-C18-diacid/18K (SEQ ID NO:26 2×AEEAc+yGlu-C18-diacid/18K), or a functional variant thereof, wherein said variant has 1 or 2 individual amino acid substitutions.

105. The GIP analogue according to claim 86, wherein said peptide is XGTFISEYSIAibLEKIKQQEFVEWLLAQKPSSGAPPPS-C16-diacid/18K (SEQ ID NO:25 C16-diacid/18K), or a functional variant thereof, wherein said variant has 1 or 2 individual amino acid substitutions.

106. A method of treatment of a condition selected from the group consisting of metabolic syndrome, obesity, pre-diabetes, type I diabetes, type 2 diabetes, insulin resistance, elevated fasting glucose, hyperglycemia, elevated fasting serum triglyceride levels, low levels of very low-density lipoprotein (VLDL), low high-density lipoprotein (HDL) levels, dyslipidemia, increased/decreased low-density lipoprotein (LDL), high cholesterol levels, abnormal deposition of lipids, a cardiovascular disease, elevated blood pressure and atherosclerosis, the method comprising administration of a therapeutically effective amount of a GIP analogue according to claim 86 to an individual in need thereof.
